(19) United States
(12) Patent Application Publication
(10) Pub. No.: US 2024/0251785 A1
(43) Pub. Date: Aug. 1, 2024

(54) SMALL MOLECULE ANTAGONISTS AND AGONISTS OF ARTHROPOD KININ RECEPTORS FOR PEST CONTROL

(71) Applicant: The Texas A&M University System, College Station, TX (US)

(72) Inventors: Patricia V. Pietrantonio, College Station, TX (US); Caixing Xiong, College Station, TX (US); Dwight D. Baker, College Station, TX (US)

(21) Appl. No.: 18/558,995

(22) PCT Filed: May 5, 2021
(86) PCT No.: PCT/US2021/030896

§ 371 Date: Nov. 3, 2023

(51) Int. Cl. A01N 25/00; A01N 35/10; A01N 37/22; A01N 43/42; A01N 43/52; A01N 43/54; A01N 43/56; A01N 43/60; A01N 43/707; A01N 43/713; A01N 43/78; A01N 43/82; A01N 43/84; A01N 43/90; A01N 47/44; A01N 47/48; A01N 51/00; A01P 7/02; A01P 7/04

(52) U.S. Cl. CPC A01N 25/006; A01N 35/10; A01N 37/22; A01N 43/42; A01N 43/52; A01N 43/54; A01N 43/56; A01N 43/60; A01N 43/707; A01N 43/713; A01N 43/78; A01N 43/82; A01N 43/84; A01N 43/90; A01N 47/44; A01N 47/48; A01N 51/00; A01P 7/02; A01P 7/04

(57) ABSTRACT

Applicants have developed novel methods and compositions for controlling, treating, preventing, or ameliorating arthropod infection and identifying kinin receptor agonists and antagonists. Novel compositions include one or more active agents of small molecules antagonists or agonists of arthropod kinin receptor. Methods of preventing disease development and infestation by arthropods are disclosed as well as methods of making, using, and producing such compositions.

### FEDERAL GRANT SUPPORT

This invention was made with government support under 2016-67015-24918 awarded by the National Institute of Food and Agriculture, United States Department of Agriculture. The government has certain rights in the invention.

### FIELD OF THE DISCLOSURE

The disclosure relates to small molecules, which are effective antagonists or agonists of arthropod kinin receptors. The invention further relates to antiparasitic and insecticidal compositions comprising the small molecules as well as methods of use of the same.

### BACKGROUND

The southern cattle tick, Rhipicephalus microplus, is one of the most important livestock pests. It is the primary vector of the deadly disease agents Babesia spp. and Anaplasma spp. Despite being eradicated from the United States since 1943, this tick species is well-established in other tropical and subtropical countries and causes enormous economic losses to the local cattle industry. Cattle fever ticks (R. annulatus and R. microplus) have always been a threat to the US cattle industry owing to proximity to endemic populations in Mexico. Despite quarantine efforts, R. microplus ticks are being found at a considerable distance north of the tick eradication quarantine area and intercepting these ticks is crucial, especially because a high proportion of these ticks are resistant to permethrin. The G protein-coupled receptors (GPCRs) are the largest family of transmembrane proteins in metazoans and form a superfamily associated with widely different biological functions in some organisms. They transduce diverse extracellular signals including light, protons, hormones, neuropeptides, glutamate and lipoglycoproteins. GPCRs are viable targets as >30% of human drugs bind to this receptor superfamily. GPCRs for small neuropeptides could be utilized for pest control by finding small molecules with agonist or antagonist activity.

Insect kinins are candidate small peptides that initially were identified for their myotropic activity in the cockroach hindgut, and then proved to be pleiotropic. Kinins regulate diuresis, gut enzyme release, sugar taste perception in contact chemosensory neurons in Aedes aegypti, and tracheal clearance and air filling before ecdysis in Drosophila. The exact physiological functions of the kinin system in ticks remain unknown.

Previous studies have predicted tick kinin sequences from the R. microplus kinin precursor peptide cDNA cloned, and fourteen of the shorter kinins were synthesized and tested on the recombinant tick kinin receptor, and all were agonists. Upon binding of an agonist. GPCRs coupled with different Gα proteins trigger different downstream cellular signaling pathways and result in changes in concentration of second messengers, such as calcium ions (Ca²⁺) (receptors coupled to Gα_{q/11}, such as the kinin receptor) or cyclic adenosine monophosphate (cAMP) (Gαᵢ/Gαₛ) or regulate the activity of Rho GTPase nucleotide exchange factors (Gα_{12/13}). To identify ligands of a GPCR, monitoring the titer of the second messenger upon activation is among the most widely used methodologies. The tick kinin receptor couples to Gα_{q/11} protein.

High-throughput screening (HTS) of a target GPCR is one method by which to discover new drugs. However, chemical library selection for HTS on a tick neuropeptide GPCR is problematic owing to the great evolutionary differences between invertebrates and humans. Screening of drugs that are active on the most similar human receptors often yields low activity in the arthropod receptor counterpart, as was previously shown by screening an antagonist library of the human neurokinin receptors on the tick kinin receptor, as they are not orthologs.

As peptides are expensive to mass-produce and susceptible to enzymatic degradation, and disease prevention relies heavily on arthropod control, and as a consequence of the increasing acaricide resistance detected worldwide, there is an urgent need for novel, cheaper, mass-producible, and stable compounds and compositions for arthropod control.

### SUMMARY

The present disclosure relates to small molecule antagonists and/or agonists for arthropod kinin receptors. The compounds and compositions may be used for treatment and control of parasitic infestations on animals. Further, the compositions may be used to prevent the spread of disease through parasites, particularly ticks and mosquitoes. The compounds may also be used in identifying the function of arthropod kinin receptors.

The small molecules may be formulated into pharmaceutically acceptable antiparasitics. In another embodiment the small molecules may be combined with one or more additional compounds to make the antiparasitic. The pharmaceutically acceptable antiparasitic may be administered to subjects in need thereof. Preferably administration is topical. More preferably, administration is to the body surface, for example by dipping or misting the subject. In an alternate preferred embodiment, administration may be applied to items worn by or shelters used by the subject.

The small molecules may be used in sugar baits against dipterans such as mosquitoes, in push-pull strategies. Due to the actions seen in the gut, it is predicted that the small molecule kinin antagonists will prolong the feeding time on the sugar mixture containing another insecticide, increasing the amount of insecticide ingested in a single bout. This could be also used against Drosophilid flies such as Drosophila suzukii or other flies. Baits with small molecule agonists in a sugar mixture are predicted to push the female mosquito as shown by the peptide kinin agonist 1728 (Kwon et al., 2016, PNAS, herein incorporated by reference in its entirety).

The small molecules may be formulated into an agricultural antiparasitic to prevent or control the spread of the parasites. The antiparasitic may then be applied to one or more plants. The application may be to any part of the plant. Preferably the application to the plant is to the shoot system. The plants may be in a park, yard, field, or lot. In a preferred embodiment, application to a plant may be formulated against phytophagous mites, such as, but not limited to, spider mites or other mites belonging to the subclass Acari, family Eriophidae, Tetranychidae, and/or Tenuipalpidae.

The small molecules or pharmaceutical compositions thereof may also be administered to the parasite.

The small molecules may also be used in assays. The assay may be cell, tissue, organ, system, or whole animal-based assays. In preferred embodiments the assay is cell-based. In a more preferred embodiment, the assay is a dual-addition calcium fluorescence assay. In another preferred embodiment, the assay is an organ-based assay.

In some embodiments, additional molecules may be created/identified. Any arthropod kinin may be used as a control kinin to elucidate the small molecule analogs, which can be determined through a dual-addition calcium fluorescence assay. In a preferred embodiment, the determination is done in a high throughput screening assay. In another embodiment, the determination may be done individually. In yet other embodiments, the determination may be done in groups. In a preferred embodiment, the agonists will have a normalized percent activation of greater than about 40% as defined by Equation (II) (defined herein). In another preferred embodiment, the antagonists will have an inhibitory activity greater than about 40% as defined by Equation (III) (defined herein).

The small molecule analogs may also be used for in silico screening of chemical libraries for virtual hits having a similar structure. In a preferred embodiment, the virtual hits have a Tanimoto structural similarity of greater than about 50%.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 shows the workflow for the discovery of novel small molecules ligands of the kinin receptor from the southern cattle tick, Rhipicephalus microplus. (1) A high-throughput calcium fluorescence assay was developed with the recombinant tick kinin receptor-expressing cells (BMLK3) in 384-well plates; (2) A ‘dual-addition’ cell assay was developed to allow identification of agonist and antagonist molecules in the same assay; (3) Both experimentally obtained and virtually predicted hit molecules were validated using 20 concentrations in the fluorescence assay; (4) Validation of hit molecules was by obtaining their response curves and analyzed by comparison of their IC₅₀s; (5) Structural activity relationship (SAR) analyses were performed for hit molecules with high structural similarity; (6) Potent hit molecules were tested in vivo using a canonical hindgut contraction inhibition assay.

FIG. 2 shows a stepwise summary of the HTS results using BMLK3 cells and vector-only (V/O, control) CHO-K1 cells.

FIG. 3A is a graphical representation of the dose-response curves of representative HTS hit molecule for a full antagonist, which completely inhibited the calcium response to the kinin peptide. FIG. 3B is a graphical representation of the dose-response curves of representative HTS hit molecule for a partial antagonist, which partially inhibited the calcium response to the kinin peptide but did not reach full inhibition even at highest concentration of 25 μmol L⁻¹. FIG. 3C is a graphical representation of the dose-response curves of representative HTS hit molecule for a false (nonspecific) agonist, which elicited the same agonistic dose-response in both BMLK3 and V/O cells. FIG. 3D is a graphical representation of the dose-response curves of representative HTS hit molecule for a false antagonist, which did not inhibit the agonist peptide in a dose-response fashion. Hit molecules were tested in a dose-response assay on both recombinant tick kinin receptor cells (BMLK3, left two columns) and the cells transformed only with the vector plasmid (V/O, right two columns). The first and third columns show the calcium fluorescence responses after the assay first addition of test compounds. Each compound was tested through a dilution series of 20 dosages (dilution factor 1:1.4), ranging from 28 nmol L⁻¹ to 25 μmol L⁻¹ as final concentrations. The second and fourth columns show the respective calcium responses 5 min after the assay second addition of the tick kinin receptor agonist peptide. Rhimi-K-1 (QFSPWGamide, 1 μmol L⁻¹). Cell calcium responses in relative fluorescence units (RFU) were recorded using a Clariostar plate reader (BMG Technology®). The dose-response curves were calculated by Graphpad PRISM software using nonlinear regression [log (inhibitor/agonist) versus response-variable slope (four parameters)].

FIG. 4A shows the dose-response curve of the full agonist SACC-0064443. FIG. 4B shows the dose-response curve of the full agonist SACC-0053274. FIG. 4C shows the dose-response curve of the full agonist SACC-0048555. FIG. 4D shows the dose-response curve of the full agonist SACC-0125715. FIG. 4E shows the dose-response curve of the full agonist SACC-0024648. FIG. 4F shows the dose-response curve of the full agonist SACC-0113072. FIG. 4G shows the dose-response curve of the full agonist SACC-0105544. FIG. 4H shows the dose-response curve of the full agonist SACC-0018618. FIG. 4I shows the dose-response curve of the full agonist SACC-0126713. FIG. 4J shows the dose-response curve of the full agonist SACC-0099442. FIG. 4K shows the dose-response curve of the full agonist SACC-0015411. These are the 11 most potent full antagonists from an in-house SAC library screening. Hit molecules identified by the experimental HTS, shown in FIGs. A-C and E-I, and by virtual predictions, shown in FIGs. D. J, and K, were validated through a dose-response assay. Each compound was tested at 20 concentrations from 25 μmol L⁻¹ to 28 nmol L⁻¹, obtained through serial dilutions (1:1.4 dilution factor) as final concentrations in the cell media. Compounds were added into cell media and incubated with the cells for 5 min, followed by the assay second addition of the tick kinin receptor agonist peptide. Rhimi-K-1 (QFSPWGamide, 1 μmol L⁻¹). The calcium responses in relative fluorescence units (RFU) at 5 min after the second addition were measured by the Clariostar plate reader (BMG Technology®). The dose-response curves were generated by Graphpad PRISM software using nonlinear regression [log(inhibitor) versus response-variable slope (four parameters)]. Although 27 of the hit molecules showed dose-dependent antagonistic activity on the recombinant tick kinin receptor (18 antagonists in Table 3 and II antagonists in Table 4), herein only the curves for the 11 most potent hit molecule full antagonists (IC₅₀<10 μmol L⁻¹) are shown.

FIG. 5A shows dose-response curves for the most potent antagonist SACC-0064433. FIG. 5B shows dose-response curves for SACC-0412062, a structural analog to SACC-0064443 as identified from in silico searches having 95% structural similarity.

FIG. 5C shows dose-response curves for SACC-0412060, a structural analog to SACC-0064443 as identified from in silico searches having 95% structural similarity. FIGURE SD shows dose-response curves for SACC-0412066, a structural analog to SACC-0064443 as identified from in silico searches having 95% structural similarity. FIG. 5E shows dose-response curves for SACC-0412065, a structural analog to SACC-0064443 as identified from in silico searches having 95% structural similarity. FIG. 5F shows dose-response curves for SACC-0412061, a structural analog to SACC-0064443 as identified from in silico searches having 95% structural similarity. FIG. 5G shows dose-response curves for SACC-0412064, a structural analog to SACC-0064443 as identified from in silico searches having 95% structural similarity. FIG. 5H shows dose-response curves for SACC-0412063, a structural analog to SACC-0064443 as identified from in silico searches having 95% structural similarity. For each compound, receptor-expressing cells (BMLK3) were preincubated 5 min with each of 20 concentrations from 25 μmol L⁻¹ to 28 nmol L⁻¹, obtained through serial dilutions (1:1.4 dilution rate). Post-incubation, a tick kinin receptor agonist peptide, Rhimi-K-1 (QFSPWGamide, 1 μcool L⁻¹), was added to the cell media, and after 5 min the cell calcium responses in relative fluorescence units (RFU) were recorded by a Clariostar plate reader (BMG Technology®). The curves were generated by Graphpad PRISM software using nonlinear regression [log(inhibitor) versus response-variable slope (four parameters)]. The compounds in FIGs. A-D were full antagonists; in FIGs. E and F partial antagonists; and in FIGs. G and H had low antagonist activity.

FIG. 6 shows a structure-activity relationships (SAR) analysis of a validated antagonist (SACC-0064443) derived from the I-ITS, and of seven analogs of the same molecule identified by in silico searches of the available libraries. The structure on the left depicts the backbone of these eight structurally similar molecules. The change of an aromatic group (R) caused different antagonistic activity. The top four molecules [aromatic group(s) in blue] acted as full antagonists of the tick kinin receptor, and the bottom molecules acted as either partial antagonists (SACC-0412065, SACC-0412061) or very weak partial antagonists (SACC-0412063 and SACC-0412064) (dose-response curves of these eight molecules are in FIG. 5).

FIG. 7A shows a test of potent antagonist hit molecules on the recombinant kinin receptor from mosquito Aedes aegypti (IGKN G12, CHO-K1 cells) in the calcium fluorescence assay. FIG. 7B shows a test of potent antagonist hit molecules on the recombinant tick kinin receptor (BMLK3). SACC-0412060, SACC-0412002 and SACC-0412066 were among the most potent antagonists. SACC-0412064 had a very weak antagonistic activity and, therefore, was used as a negative control. Each molecule was tested at three different dosages (10, 30 and 100 μmol L⁻¹ as final concentration). For testing each of these final concentrations, first, a 10× small molecule stock solution was added to the cell media. After 5 min of incubation with the compound, a 3× kinin agonist analog 1728 ([Aib]FF[Aib]WGamide) solution was added to reach a final concentration of 1 μmol L⁻¹. After this second addition, calcium fluorescence responses (in relative fluorescence units, RFU) of the cells were immediately recorded for 60 s with 3-s intervals between reads using a Clariostar plate reader (BMG Technology®). The Y-axis value was calculated as the percentage of average RFU of each treatment to the RFU of the control (solvent in first addition and the kinin agonist analog 1728 in second addition=solvent+1728), as the cell response of the control well was regarded as 100% response in each test. Three replicates were done for each treatment (n=3, mean±SEM).

FIG. 8A shows an in vivo validation of antagonists of kinin receptors in the arthropod hindgut contraction inhibition assay in isolated hindguts from females of the mosquito Aedes aegypti were preincubated with 100 μmol L⁻¹ of antagonist SACC-0412000 or SACC-0412066, the negative control small molecule (SACC-0412064), or solvent control for 5 min. Subsequently, 10 μmol L⁻¹ of the kinin agonist analog 1728 ([Aib]FF[Aib]WGamide) was added. Tissues were filmed for 60 s both before treatment and at 1 h post-treatment using an Olympus ZS60 microscope. The fold-change in the contraction rate in 60 s was calculated by dividing the post-treatment contraction rate by the basal contraction rate; each dot represents one hindgut contraction assay (n=30 for each treatment). (A) Differences in the means of change of contraction rate were analyzed by the Kruskal-Wallis (P<0.05) test followed by Dunn's multiple comparisons test (P<0.05) using Graphpad PRISM software. Black lines above results and ‘n.s.’ indicate no statistical difference between groups. FIG. 8B shows the differences in the change of contraction rate between SACC-0412062 and solvent with the kinin agonist analog 1728 were analyzed by the nonparametric Mann-Whitney U-test, ****, P<0.0001 using the same validation. (The annotation of the small molecules on the X-axes displays only four digits of their full name owing to space limitations, and the prefix ‘SACC-041’ should be added).

### DETAILED DESCRIPTION

So that the present disclosure may be better understood, certain terms are first defined.

As used herein, “weight percent”, “wt. %”, “percent by weight”, “% by weight”, and variations thereof refer to the concentration of a substance as the weight of that substance divided by the total weight of the composition and multiplied by 100. It is understood that, as used here, “percent”, “%”, and the like are intended to be synonymous with “weight percent,” “wt.-%,” etc.

As used herein, the term “about” refers to variation in the numerical quantity that can occur, for example, through typical measuring and liquid handling procedures used for making concentrates or use solutions in the real world, through inadvertent error in these procedures; through differences in the manufacture, source, or purity of the ingredients used to make the compositions or carry out the methods, and the like. The term “about” also encompasses amounts that differ due to different equilibrium conditions for a composition resulting from a particular initial mixture. Whether or not modified by the term “about”, the claims include equivalents to the quantities.

It should be noted that, as used in this specification and the appended claims, the singular forms “a,” “an,” and “the” include plural referents unless the content clearly dictates otherwise. Thus, for example, reference to a composition containing “a compound” includes a composition having two or more compounds. It should also be noted that the term “or” is generally employed in its sense including “and/or” unless the content clearly dictates otherwise.

“Small molecule” as used herein, unless otherwise indicated, means a synthetic small molecule that acts as an agonist or antagonist of an arthropod kinin receptor.

“Animal”, “subject”, or “patient” as used herein, unless otherwise indicated, are used interchangeably, and refers to an individual subject, and said individual subject is a mammal. Specifically, mammal refers to a vertebrate animal that is human or non-human, which are members of the taxonomic class Mammalia. Non-exclusive examples of non-human mammals include companion animals and livestock. Non-exclusive examples of a companion animal include dog (canine), cat (feline), llama (camelid), and horse (equine). Preferred companion animals are dog, cat, and horse. More preferred is dog or cat. Non-exclusive examples of livestock include pigs (porcine), camel, rabbits, goat (caprine), sheep (ovine), deer, elk, cattle (bovine), camelid, and bison. Preferred livestock is cattle.

“Infestation”, as used herein, unless otherwise indicated, refers to the state or condition of having parasites on the body and/or in the body. Furthermore, the infestation may lead to an infection on or in the subject, which may be microbial, viral, or fungal.

“Parasite(s)”, as used herein, unless otherwise indicated, refers to ectoparasites. Ectoparasites are organisms of the Arthropoda phylum (arachnids and insects) which feed through or upon the skin of its host. Preferred arachnids are of the order Acarina (acarines), e.g., ticks and mites. Preferred insects are of the Order Diptera which include biting or myiasis-inducing flies (midges, mosquitos, stable fly, horn fly, blow fly (e.g., Cochliomyia), horse fly, sand fly, and the like), Siphonaptera (fleas), and Phthiraptera (lice). Parasites also encompasses the different life stages of the ectoparasite, including eggs, pupae, and larvae which feed on or in the body. Parasite(s) also encumbers endoparasites, parasites that live within the body of its host and include helminths (e.g., trematodes, cestodes, and nematodes) and protozoa.

As used herein, the term “administration” refers to the introduction of a composition into a subject by a chosen route. Administration can be local or systemic. The compositions utilized in the methods described herein can be administered, for example, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraocularly, orally, topically, locally, by inhalation, by injection, by infusion, by continuous infusion, by localized perfusion bathing target cells directly, by catheter, by lavage, by gavage, in cremes, or in lipid compositions. The preferred method of administration can vary depending on various factors (e.g., the components of the composition being administered and the severity of the condition being treated). For example, if the chosen route is intravenous, the composition (such as a composition including a disclosed immunogen) is administered by introducing the composition into a vein of the subject.

“Therapeutically effective amount”, as used herein, unless otherwise indicated, refers to an amount of one of small molecules of the present invention that (i) treat or prevent the particular parasitic infestation, (ii) attenuates, ameliorates, or eliminates one or more symptoms of the particular parasitic infestation, or (iii) prevents or delays the onset of one or more symptoms of the particular parasitic infestation described herein.

“Treatment”. “treating”, and the like, as used herein, unless otherwise indicated, refers to reversing, alleviating, or inhibiting the parasitic infestation. As used herein, these terms also encompass, depending on the condition of the subject preventing the onset of a disorder or condition, or of symptoms associated with a disorder or condition, including reducing the severity of a disorder or condition or symptoms associated therewith prior to affliction with said infestation. Thus, treatment can refer to administration of the composition of the present invention to a subject that is not at the time of administration afflicted with the parasitic infestation, for example, as prophylactic treatment. Treating also encompasses preventing the recurrence of an infestation or of symptoms associated therewith as well as references to “control” (e.g., kill, repel, expel, incapacitate, deter, eliminate, alleviate, minimize, and eradicate).

“Pharmaceutically acceptable” as used herein, unless otherwise indicated, suggests that the substance or composition must be compatible chemically and/or toxicologically with the other ingredients comprising the composition and/or the subject being treated therewith.

The term “effective amount” in connection with a small molecule means an amount capable of treating or preventing a disorder, disease or condition, or symptoms thereof, disclosed herein.

The term “combination” or administration “in combination” includes administration as a mixture, simultaneous administration using separate formulations, and consecutive administration in any order.

As used herein and unless otherwise specified, an “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 or carbon atoms. Representative alkyl groups include -methyl, -ethyl, -n-propyl, -n-butyl, -n-pentyl and -n-hexyl while saturated branched alkyls include -isopropyl, -secbutyl, -isobutyl, -tert-butyl, -isopentyl, -neopentyl, tert-pentyl, -2-methylpentyl, -3-methylpentyl, -4-methylpentyl, -2,3-dimethylbutyl and the like. An “alkenyl” group is an alkyl group that contains one or more carbon-carbon double bonds. An “alkynyl” group is an alkyl group that contains one or more carbon-carbon triple bonds. Examples of unsaturated alkyl groups include, but are not limited to, vinyl, allyl, —CH═CH(CH3), —CH═C(CH3)2, —C(CH3)═CH2, —C(CH3)CH(CH3), —C(CH2CH3)═CH2, —C≡C(CH3), —C≡C(CH2CH3), —CH2C≡CH, —CH2C≡C(CH3), and —CH2C≡C(CH2CH3), among others. An alkyl group can be substituted or unsubstituted. When the alkyl groups described herein are said to be “substituted,” they may be substituted with any substituent or substituents as those found in the exemplary compounds and embodiments disclosed herein, as well as halogen; hydroxy; alkoxy; cycloalkyloxy, aryloxy, heterocyclyloxy, heteroaryloxy, heterocycloalkyoxy, cycloalkylallyloxy, aralkyloxy, heterocyclylalkyloxy, heteroarylalkyloxy, heterocycloalkyalkyloxy; oxo (═O); amino, alkylamino, cycloalkylamino, arylamino, heterocyclylamino, heteroarylamino, heterocycloalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)2.

As used herein and unless otherwise specified, a “cycloalkyl” group is a saturated, or partially saturated cyclic alkyl group of from 3 to 10 carbon atoms having a single cyclic ring or multiple condensed or bridged rings which can be optionally substituted. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantly and the like. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

As used herein and unless otherwise specified, an “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryl groups include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

As used herein and unless otherwise specified, a “heteroaryl” group is an aromatic ring system having one to four heteroatoms as ring atoms in a heteroaromatic ring system. % herein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur, and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indol-2-onyl), isoindolin-1-onyl, azaindolyl, pyrrolopyridyl (e.g., 1H-pyrrolo[2,3-b]pyridyl), indazolyl, benzimidazolyl (e.g., 1H-benzo[d]imidazolyl), azabenzimidazolyl, imidazopyridyl (e.g., 1H-imidazo[4,5-b]pyridyl), pyrazolopyridyl, triazolopyridyl, benzotriazolyl (e.g., 1H-benzo[d] [1,2,3]triazolyl), benzoxazolyl (e.g., benzo [d]oxazolyl), benzothiazolyl, benzothiadiazolyl, isoxazolopyridyl, thianaphthalenyl, purinyl, xanthinyl, adeninyl, guaninyl, quinolinyl, isoquinolinyl, 3,4-dihydroisoquinolin-1 (2H)-onyl, tetrahydroquinolinyl, quinoxalinyl, and quinazolinyl groups. A heteroaryl group can be substituted or unsubstituted.

As used herein and unless otherwise specified, a “heterocyclyl” is an aromatic ring system (also referred to as heteroaryl) or non-aromatic cycloalkyl (also referred to as heterocycloalkyl) in which one to four of the ring carbon atoms are independently replaced with a heteroatom. Suitable heteroatoms include oxygen, sulfur, and nitrogen. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. Heterocyclyl groups encompass unsaturated, partially saturated and saturated ring systems, such as, for example, imidazolyl, imidazolinyl and imidazolidinyl (e.g., imidazolidin-4-onyl or imidazolidin-2,4-dionyl) groups. The phrase heterocyclyl includes fused ring species, including those comprising fused aromatic and non-aromatic groups, such as, for example, 1- and 2-aminotetraline, benzotriazolyl (e.g., 1H-benzo[d][1,2,3]triazolyl), benzimidazolyl (e.g., 1H-benzo[d]imidazolyl), 2,3-dihydrobenzo[1,4]dioxinyl, and benzo[1,3]dioxolyl. The phrase also includes bridged polycyclic ring systems containing a heteroatom such as, but not limited to, quinulidyl. Representative examples of a heterocyclyl group include, but are not limited to, aziridinyl, azetidinyl, azepanyl, oxetanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, benzisoxazolyl (e.g., benzo [d]isoxazolyl), thiazolyl, thiazolinyl, isothiazolyl, thiadiazolyl, oxadiazolyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dioxyl, dithianyl, pyranyl, pyridyl, pyrimidyl, pyridazinyl, pyrazinyl, triazinyl, dihydropyridyl, dihydrodithionyl, dihydrodithionyl, 1,4-dioxaspiro[4.5]decanyl, homopiperazinyl, quinuclidyl, indolyl (e.g., indol-2-onyl), isoindolin-1-onyl, indolinyl, isoindolyl, isoindolinyl, azaindolyl, pyrrolopyridyl (e.g., 1H-pyrrolo[2,3-b]pyridyl), indazolyl, indolizinyl, benzotriazolyl (e.g. 1H-benzo[d][1,2,3]triazolyl), benin_iidazolyl (e.g., 1H-benzo[d]imidazolyl or 1H-benzo[d]imidazol-2(3H)-onyl), benzofuranyl, benzothiophenyl, benzothiazolyl, benzoxadiazolyl, benzoxazinyl, benzodithiinyl, benzoxathiinyl, benzothiazinyl, benzoxazolyl (e.g., benzo[d]oxazolyl), benzothiazolyl, benzothiadiazolyl, benzo[1,3]d)oxolyl, pyrazolopyridyl (e.g., 1H-pyrazolo[3,4-b]pyndyl, 1H-pyrazolo[4,3-b]pyridyl), azabenzimidazolyl, imidazopyridyl (e.g., 1H-imidazo [4,5-b]pyridyl), triazolopyridyl, isoxazolopyridyl, purinyl, xanthinyl, adeninyl, guaninyl, quinolinyl, isoquinolinyl, 3,4-dihydroisoquinolin-1 (2H)-onyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, tetrahydropyrimidin-2(1H)-one and tetrahydroquinolinyl groups. Representative non-aromatic heterocyclyl groups do not include fused ring species that comprise a fused aromatic group. Examples of non-aromatic heterocyclyl groups include aziridinyl, azetidinyl, azepanyl, pyrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro [4.5]decanyl, homopiperazinyl, quinuclidyl, or tetrahydropyrimidin-2(1H)-one. Representative substituted heterocyclyl groups may be mono-substituted or substituted more than once sue˜ as, but not limited to, pyridyl or morpholinyl groups: which are 2-, 3-, 4-, 5-, or 6-substituted, or disubstituted with various substituents such as those listed below.

As used herein and unless otherwise specified, a “cycloalkylalkyl” group is a radical of the formula: -alkylcycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopropylmethyl, cyclobutylmethyl, cyclopentylmethyl, cyclohexylmethyl, cyclopropylethyl, cyclobutylethyl, cyclopentylethyl, cyclohexylethyl, cyclopentylpropyl, cyclohexylpropyl and the like.

As used herein and unless otherwise specified, an “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and aralkyl groups wherein the an aryl group is fused to a cycloalkyl group such as indan-4-yl ethyl.

As used herein and unless otherwise specified, a “heterocyclylalkyl” group is a radical of the formula: -alkylheterocyclyl, wherein alkyl and heterocyclyl are defined above. A “heteroarylalkyl” group is a radical of the formula: -alkyl-heteroaryl, wherein alkyl and heteroaryl are defined above. A “heterocycloalkylalkyl” group is a radical of the formula: -alkyl-heterocycloalkyl, wherein alkyl and heterocycloalkyl are defined above. Substituted heterocyclylalkyl groups may be substituted at the alkyl, the heterocyclyl, or both the alkyl and the heterocyclyl portions of the group. Representative heterocyclylalkyl groups include but are not limited to morpholin-4-yl ethyl, morpholin-4-yl propyl, furan-2-yl methyl, furan-3-yl methyl, pyridin-3-yl methyl, tetrahydrofuran-2-yl ethyl, and indol-2-yl propyl.

As used herein and unless otherwise specified, a “halogen” is fluorine, chlorine, bromine, or iodine.

As used herein and unless otherwise specified, a “hydroxyalkyl” group is an alkyl group as described above substituted with one or more hydroxy groups.

As used herein and unless otherwise specified, an “alkoxy” group is —O-(alkyl), wherein alkyl is defined above. An “alkylthio” group is —S-(alkyl), wherein alkyl is defined above.

As used herein and unless otherwise specified, an “alkoxyalkyl” group is -(alkyl)-O-(alkyl), wherein alkyl is defined above.

As used herein and unless otherwise specified, a “cycloalkyloxy” group is —O-(cycloalkyl), wherein cycloalkyl is defined above.

As used herein and unless otherwise specified, an “aryloxy” group is —O-(aryl), wherein aryl is defined above.

As used herein and unless otherwise specified, a “heterocyclyloxy” group is —O-(heterocyclyl), wherein heterocyclyl is defined above. A “heteroaryloxy” group is —O-(heteroaryl), wherein heteroaryl is defined above. A “heterocycloalkyloxy” group is —O-(heterocycloalkyl), wherein heterocycloalkyl is defined above.

As used herein and unless otherwise specified, an “amino” group is a radical of the formula: —NH2, —NH (R #), or —N(R #)2, wherein each R # is independently an alkyl, cycloalkyl, cycloalkylalkyl, aryl, aralkyl, heterocyclyl (e.g., heteroaryl or heterocycloalkyl), or heterocyclylalkyl (e.g., heteroarylalkyl or heterocycloalkylalkyl) group defined above, each of which is independently substituted or unsubstituted.

In one embodiment, an “amino” group is an “alkylamino” group, which is a radical of the formula: -NHalkyl or —N(alkyl)2, wherein each alkyl is independently defined above. The term “cycloalkylamino”, “arylamino”, “heterocyclylamino”, “heteroarylamino”, “heterocycloalkylamino”, or the like, mirrors the above description for “alkylamino” where the term “alkyl” is replaced with “cycloalkyl”, “aryl”, “heterocyclyl”, “heteroaryl”, “heterocycloalkyl”, or the like, respectively.

As used herein and unless otherwise specified, a “carboxy” group is a radical of the formula: —C(O)OH.

As used herein and unless otherwise specified, an “acyl” group is a radical of the formula: —C(O)(R #) or —C(O)H, wherein R # is defined above. A “formyl” group is a radical of the formula: —C(O)H.

As used herein and unless otherwise specified, an “amido” group is a radical of the formula: —C(O)—NH2, —C(O)—NH(R #), —C(O)—N(R #)2, —NH—C(O)H, —NH—C(O)—(R #), —N(R #)—C(O)H, or —N(R #)—C(O)—(R #), wherein each R # is independently defined above.

In one embodiment, an “amido” group is an “aminocarbonyl” group, which is a radical of the formula: —C(O)—NH2, —C(O)—NH(R #), —C(O)—N(RI)2, wherein each R # is independently defined above.

In one embodiment, an “amido” group is an “acylamino” group, which is a radical of the formula: —NHC(O)H, —NH—C(O)—(R #), —N(R #)—C(O)H, or —N(R #)—C(O)—(R #), wherein each R # is independently defined above.

As used herein and unless otherwise specified, a “sulfonylamino” group is a radical of the formula: —NHSO(R #) or —N(R #)SO(R #), wherein each R # is defined above.

As used herein and unless otherwise specified, an “ester” group is a radical of the formula: —C(O)—O—(R #) or —O—C(O)—(R #), wherein R # is defined above.

In one embodiment, an “ester” group is an “alkoxycarbonyl” group, which is a radical of the formula: —C(O)—O-(alkyl), wherein alkyl is defined above. The term “cycloalkyloxycarbonyl”, “aryloxycarbonyl”, “heterocyclyloxycarbonyl”, “heteroaryloxycarbonyl”, heterocycloalkyloxycarbonyl”, or the like, mirrors the above description for “alkoxycarbonyl” where the term “alkoxy” is replaced with “cycloalkyloxy”, “aryloxy”, “heterocyclyloxy”, “heteroaryloxy”. “heterocycloalkyloxy”, or the like, respectively.

As used herein and unless otherwise specified, a “carbamate” group is a radical of the formula: —O—C(O)—NH2, —O—C(O)—NH(R #), —O—C(O)—N(R #)2, —NH—C(O)—O—(R #), or —N(R #)—C(O)—O—(R #), wherein each R # is independently defined above.

As used herein and unless otherwise specified, a “urea” group is a radical of the formula: —NH(CO)NH2. —NHC(O)NH(R #), —NHC(O)N(R #)2, —N(R #)C(O)NH2, —N(R #)C(O)NH(R #), or —N(R #)C(O)N(R #)2, wherein each R # is independently defined above.

As used herein and unless otherwise specified, a “sulfinyl” group is a radical of the formula: —S(O)R #, wherein R # is defined above.

As used herein and unless otherwise specified, a “sulfonyl” group is a radical of the formula: —S(O)2R #, wherein R # is defined above.

As used herein and unless otherwise specified, an “aminosulfonyl” group is a radical of the formula: —SO2NH2, —SO2NH(R #), or —SO2N(R #)2, wherein each R # is independently defined above.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen; alkyl, alkenyl, alkynyl, cycloalkyl, aryl, heterocyclyl, heteroaryl, heterocycloalkyl, cycloalkylalkyl, aralkyl, heterocyclylalkyl, heteroarylalkyl, heterocycloalkylalkyl, optionally further substituted; hydroxy; alkoxy; cycloalkyloxy, aryloxy, heterocyclyloxy, heteroaryloxy, heterocycloalkyoxy, cycloalkylalkyloxy, aralkyloxy, heterocyclylalkyloxy, heteroarylalkyloxy, heterocycloalkyalkyloxy, oxo (═O); oxide (e.g., a nitrogen atom substituted with an oxide is called N-oxide); amino, alkylamino, cycloalkylamino, arylamino, heterocyclylamino, heteroarylamino, heterocycloalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), allylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)2.

Small Molecule Agonists and antagonists of Arthropod Kinin Receptors

The small molecules of this disclosure which act on the arthropod kinin receptor represent a new mode of action and act on a target not currently listed either in the Pesticide Manual (bcpc.org) or in the IRAC classification (irac-online.org).

The small molecules of this disclosure include the preferred small molecules disclosed in Tables 2 through 7 below, substituted small molecules of Tables 2 through 7, small molecules having a structural similarity of greater than about 30%, about 40%, about 50%, about 60%, about 70%, or greater to the small molecules disclosed in Tables 2-7 as calculated by the Tanimoto index, and small molecules having a normalized percent activation of activity greater than about 1%, about 5%, about 10%, about 15%, about 20%, about 25%, about 30%, about 35%, about 40%, or greater as defined by Equation II or inhibitors, activity greater than about 1%, about 5%, about 10%, about 15%, about 20%, about 25%, about 30%, about 35%, about 40%, or greater as defined by Equation III, below.

Small molecules having a normalized percent activation of activity greater than about 1%, about 5%, about 10%, about 15%, about 20%, about 25%, about 30%, about 35%, about 40%, or greater as defined by Equation II or inhibitory activity greater than about 1%, about 5%, about 10%, about 15%, about 20%, about 25%, about 30%, about 35%, about 40%, or greater as defined by Equation 111, below, may be determined by comparing the effect of their binding to an any arthropod kinin receptor agonists using the methods described herein. For example, any of the agonists disclosed in Xiong, C., et al. (The Cattle Fever Tick, Rhipicephalus microplus, as a model for Forward Pharmacology to Elucidate Kinin GPCR Function in the Acari, Front. Physiol., 10:1008) may be used as the control agonist.

Chemical structural similarity may be calculated using 2D or 3D fingerprints using any known methods. Exemplary 2D methods include, but are not limited to, MACCS keys, Obabel FP3 fingerprints, Fragment-based Daylight, BCI, or UNITY 2D fingerprints using Tanimoto index, Euclidean, Manhattan, or Mahalanobis metrics. Exemplary 3D methods include, but are not limited to, molecular shape, pharmacophore points, molecular interaction fields for structural comparisons or GETAWAY or 3D-MoRSE for chemical descriptors. Network-based algorithms may also be used, such as CSNAP or CSNAP3D. Preferably, Tanimoto index is used to calculate the similarity.

Also included are stereoisomers, E and Z isomers, tautomers, and isotopologues of the small molecules.

The small molecules include, but are not limited to:

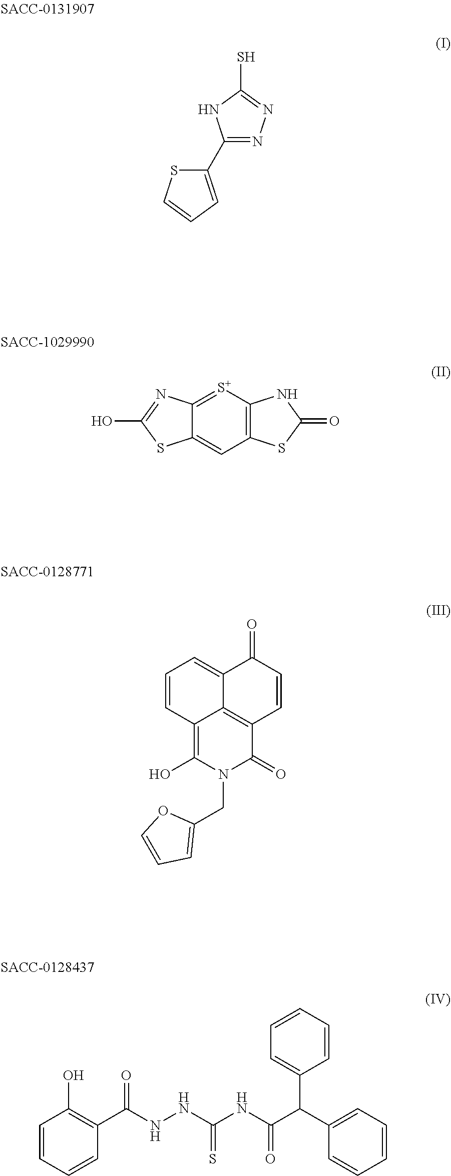 [SMILES: Sc1nnc(-c2cccs2)[nH]1 ; O=c1[nH]c2[s+]c3nc(O)sc3cc2s1 ; O=C1C=Cc2c(=O)n(Cc3ccco3)c(O)c3cccc1c23 ; O=C(NNC(=S)NC(=O)C(c1ccccc1)c1ccccc1)c1ccccc1O]

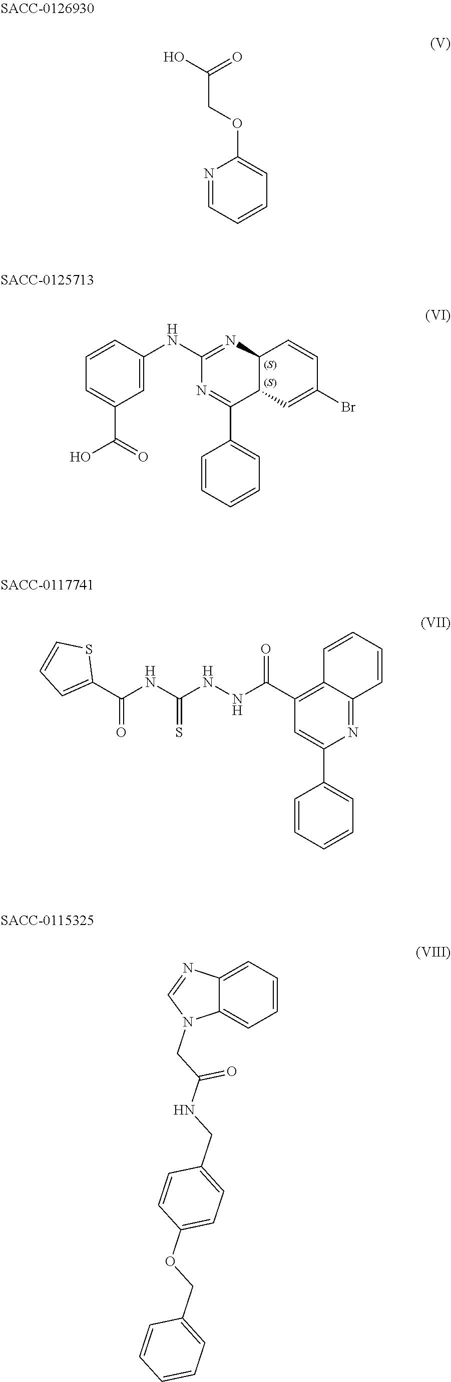

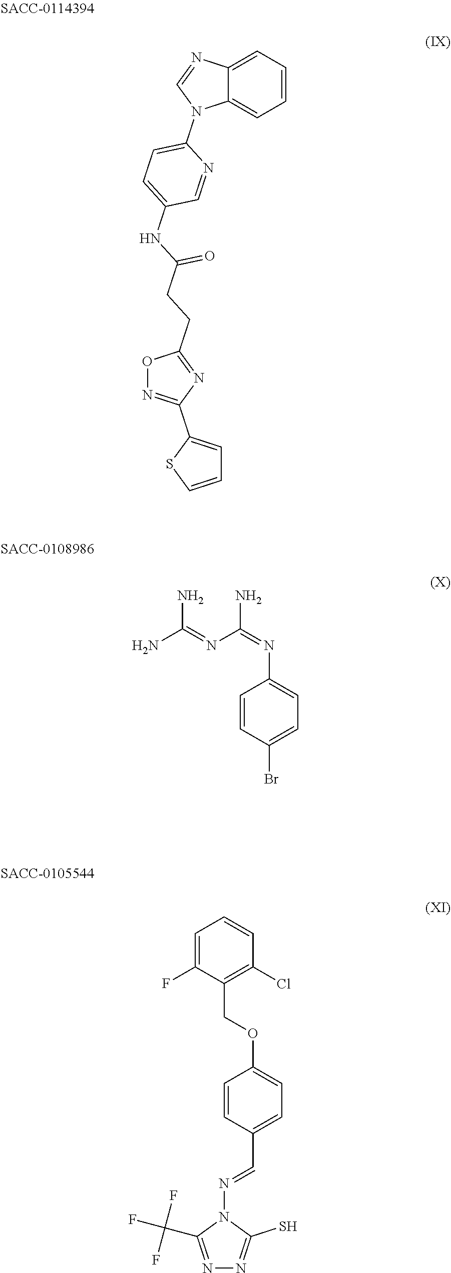 [SMILES: O=C(CCc1nc(-c2cccs2)no1)Nc1ccc(-n2cnc3ccccc32)nc1 ; NC(N)=N/C(N)=N\c1ccc(Br)cc1 ; Fc1cccc(Cl)c1COc1ccc(/C=N/n2c(S)nnc2C(F)(F)F)cc1]

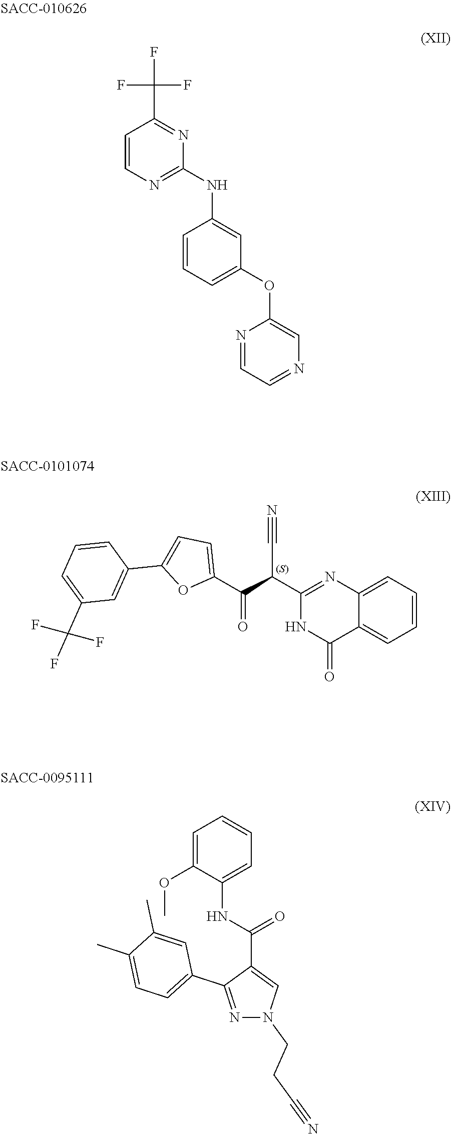

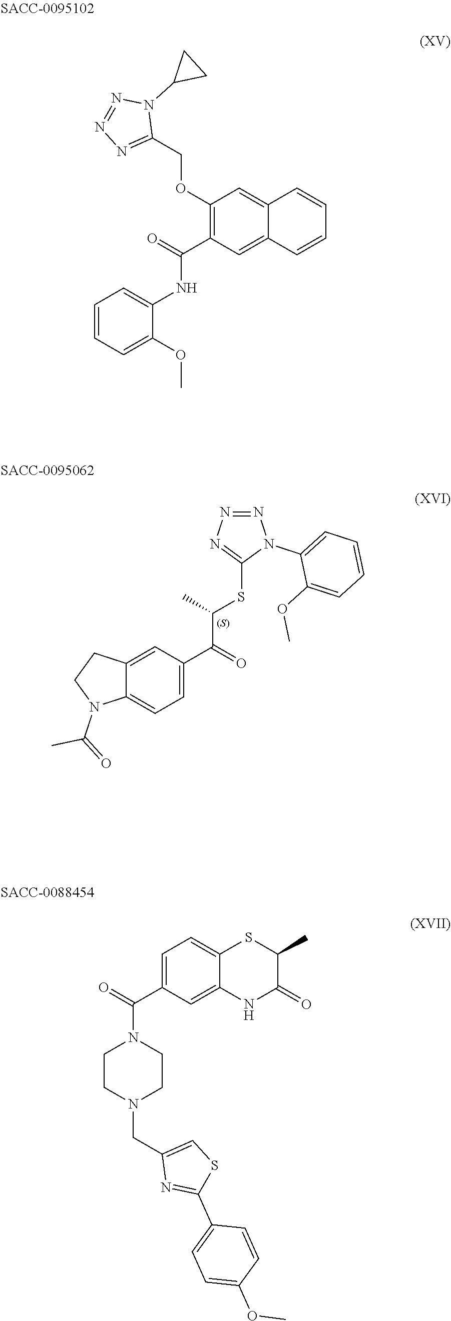

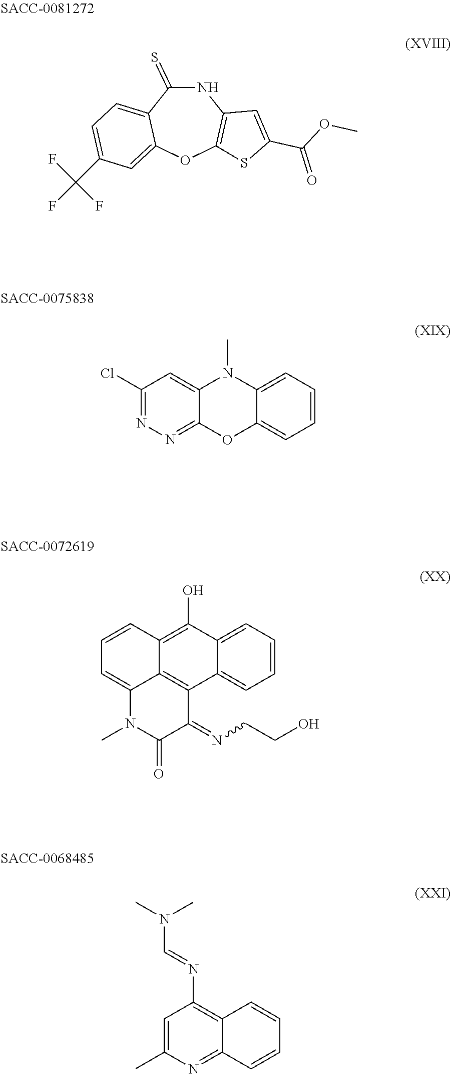

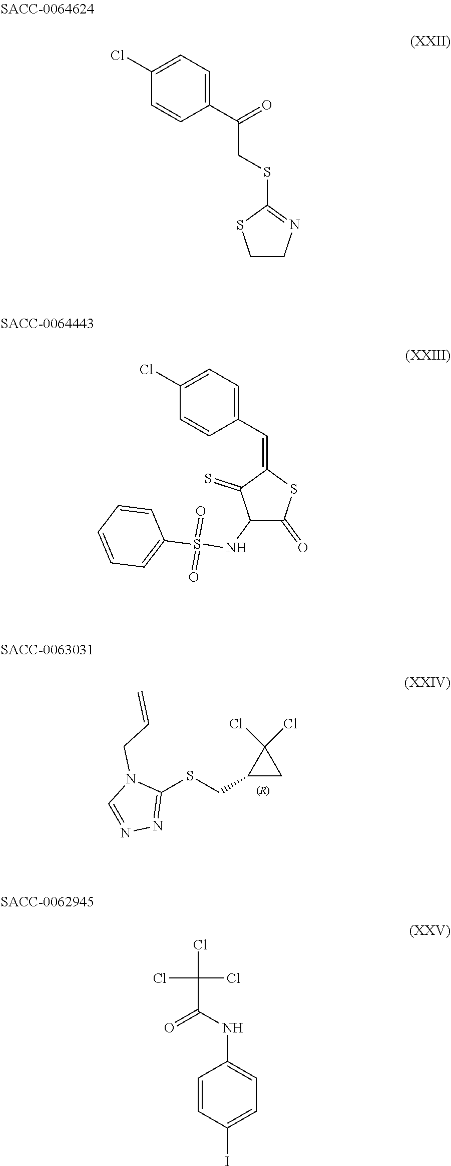

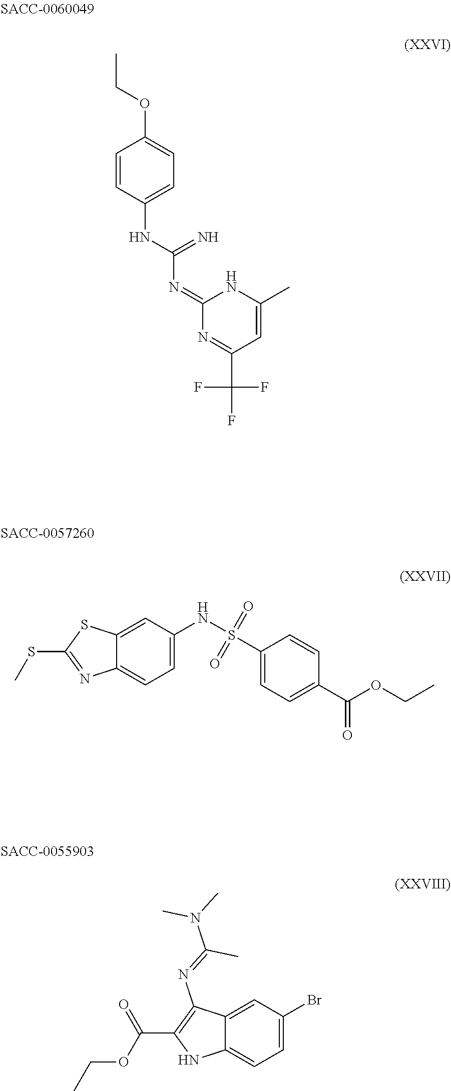

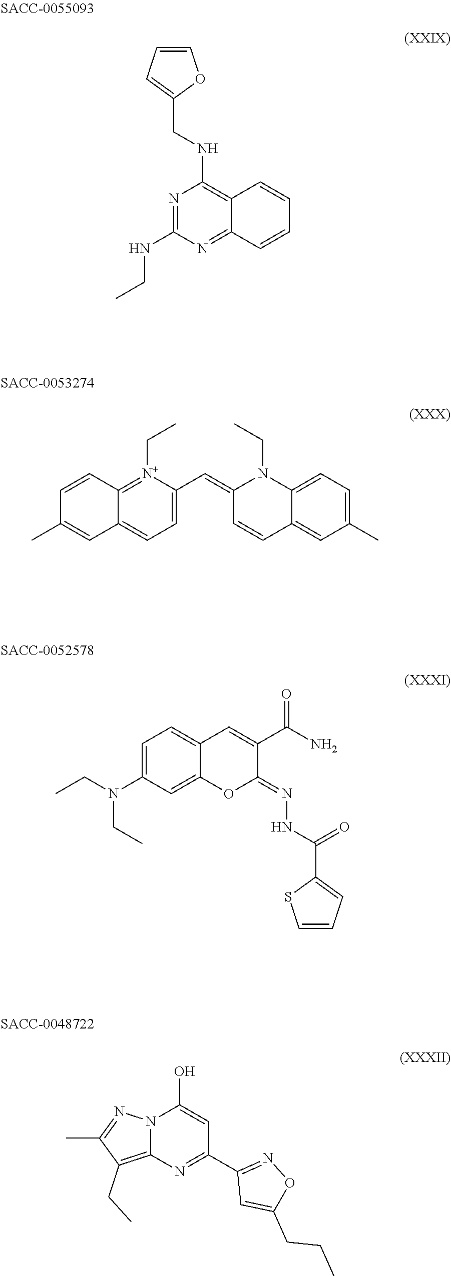

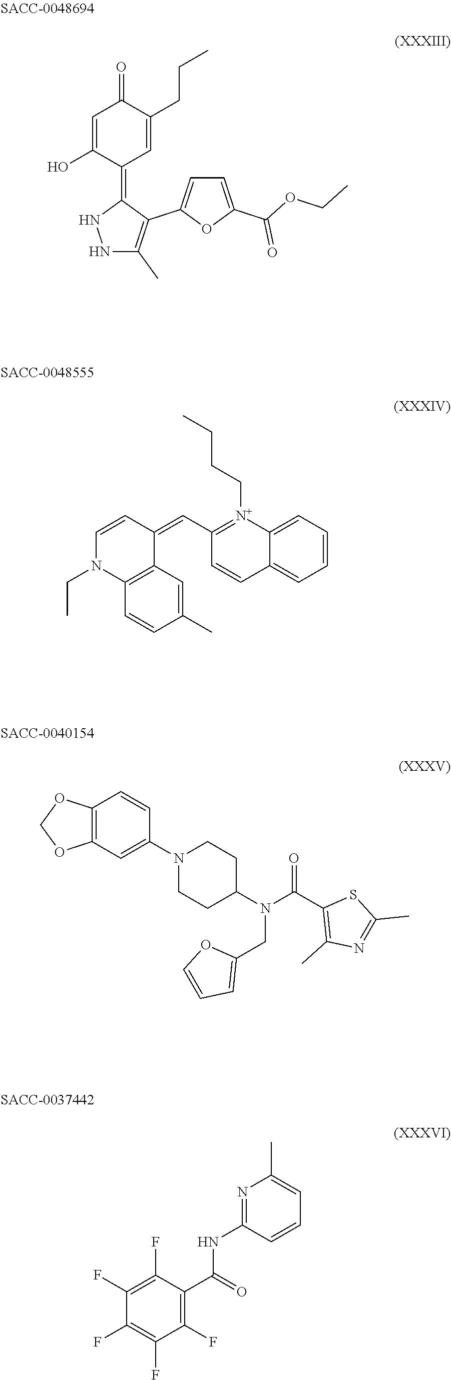 [SMILES: CCCC1=C/C(=C2/NNC(C)=C2c2ccc(C(=O)OCC)o2)C(O)=CC1=O ; CCCC[n+]1c(/C=C2/C=CN(CC)c3ccc(C)cc32)ccc2ccccc21 ; Cc1nc(C)c(C(=O)N(Cc2ccco2)C2CCN(c3ccc4c(c3)OCO4)CC2)s1 ; Cc1cccc(NC(=O)c2c(F)c(F)c(F)c(F)c2F)n1]

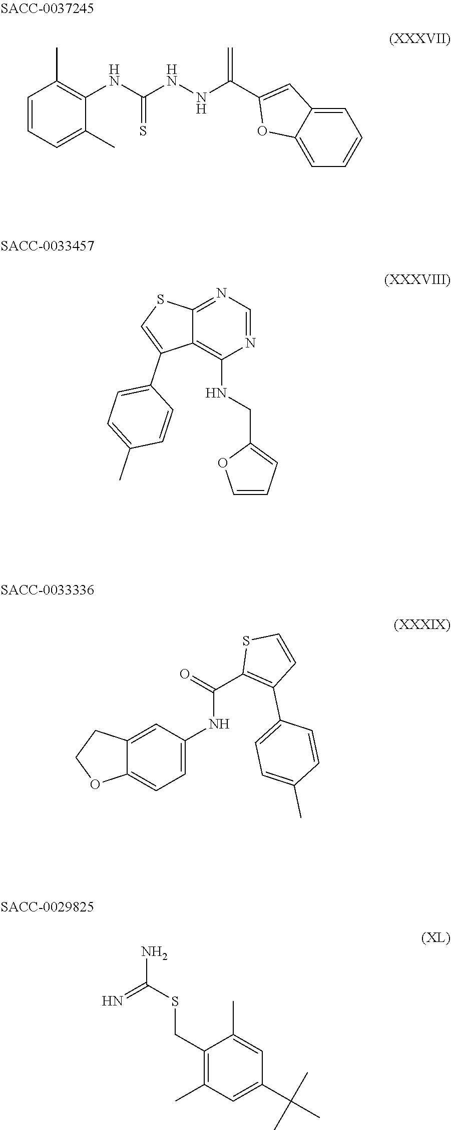 [SMILES: C=C(NNC(=S)Nc1c(C)cccc1C)c1cc2ccccc2o1 ; Cc1ccc(-c2csc3ncnc(NCc4ccco4)c23)cc1 ; Cc1ccc(-c2ccsc2C(=O)Nc2ccc3c(c2)CCO3)cc1 ; Cc1cc(C(C)(C)C)cc(C)c1CSC(=N)N]

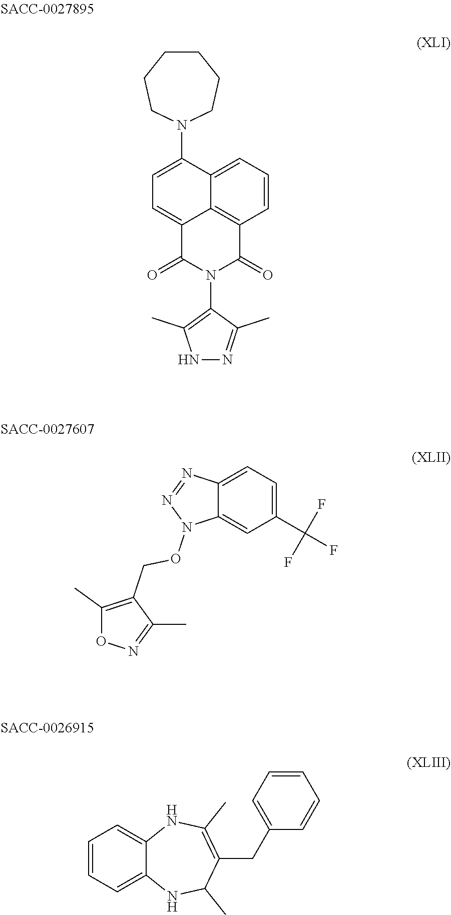 [SMILES: Cc1n[nH]c(C)c1N1C(=O)c2cccc3c(N4CCCCCC4)ccc(c23)C1=O ; Cc1noc(C)c1COn1nnc2ccc(C(F)(F)F)cc21 ; CC1=C(Cc2ccccc2)C(C)Nc2ccccc2N1]

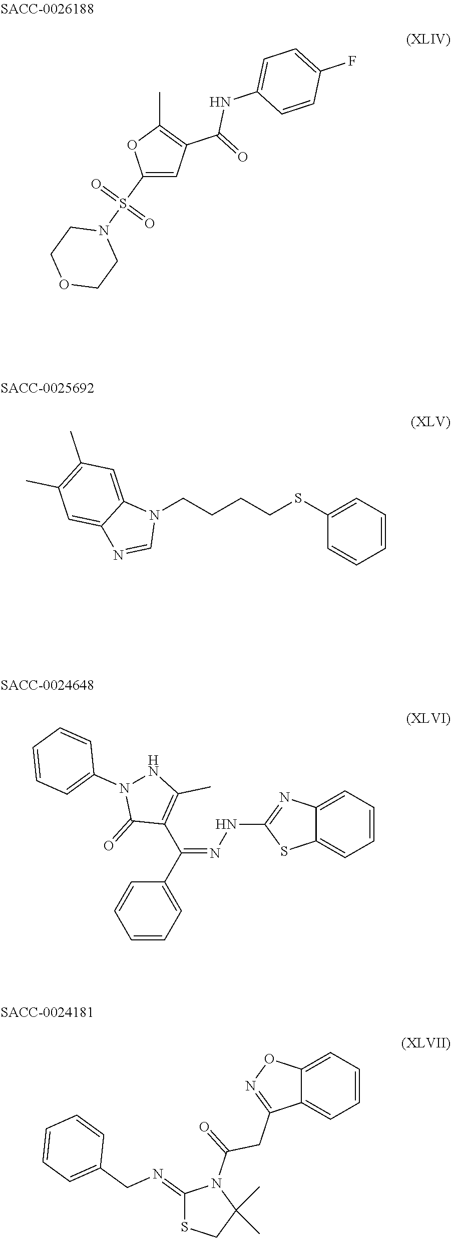 [SMILES: Cc1oc(S(=O)(=O)N2CCOCC2)cc1C(=O)Nc1ccc(F)cc1 ; Cc1cc2ncn(CCCCSc3ccccc3)c2cc1C ; Cc1[nH]n(-c2ccccc2)c(=O)c1/C(=N\Nc1nc2ccccc2s1)c1ccccc1 ; CC1(C)CS/C(=N\Cc2ccccc2)N1C(=O)Cc1noc2ccccc12]

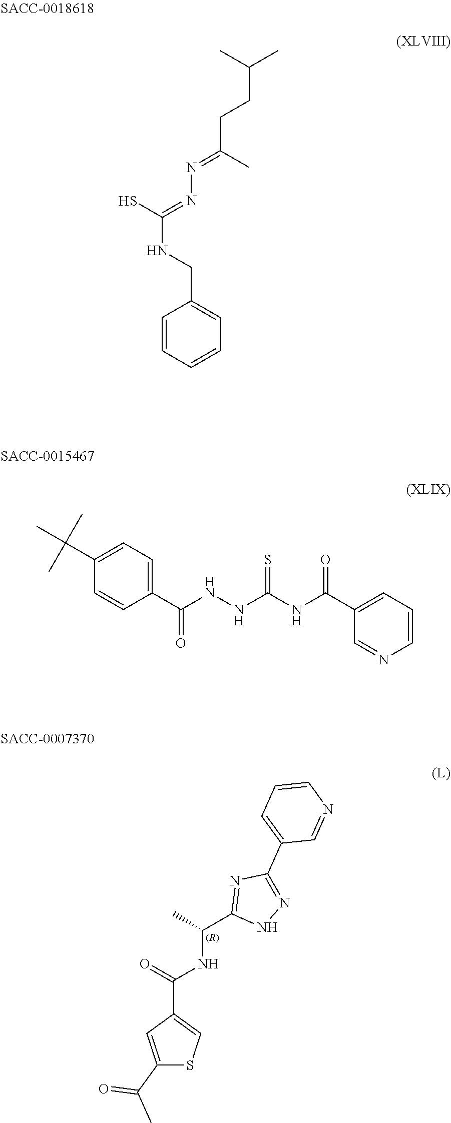

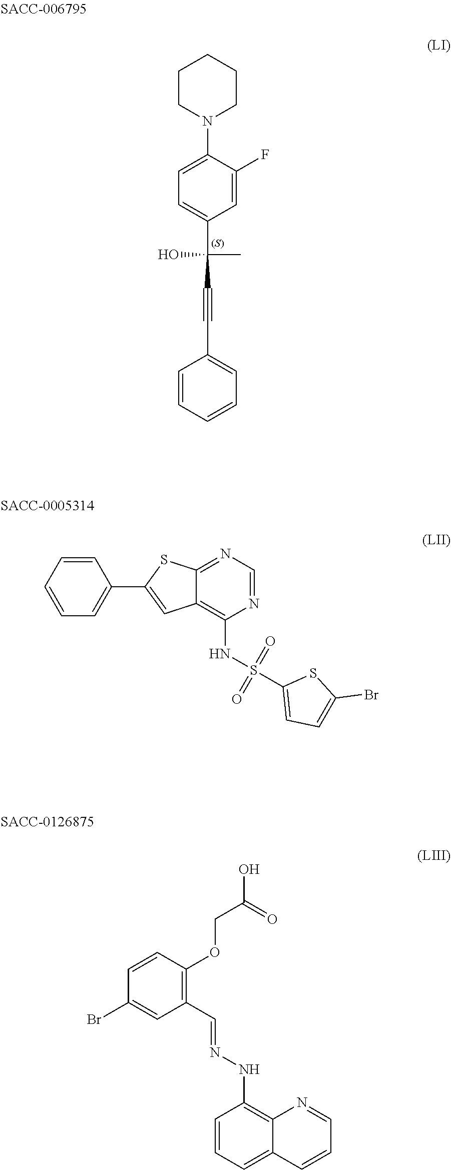

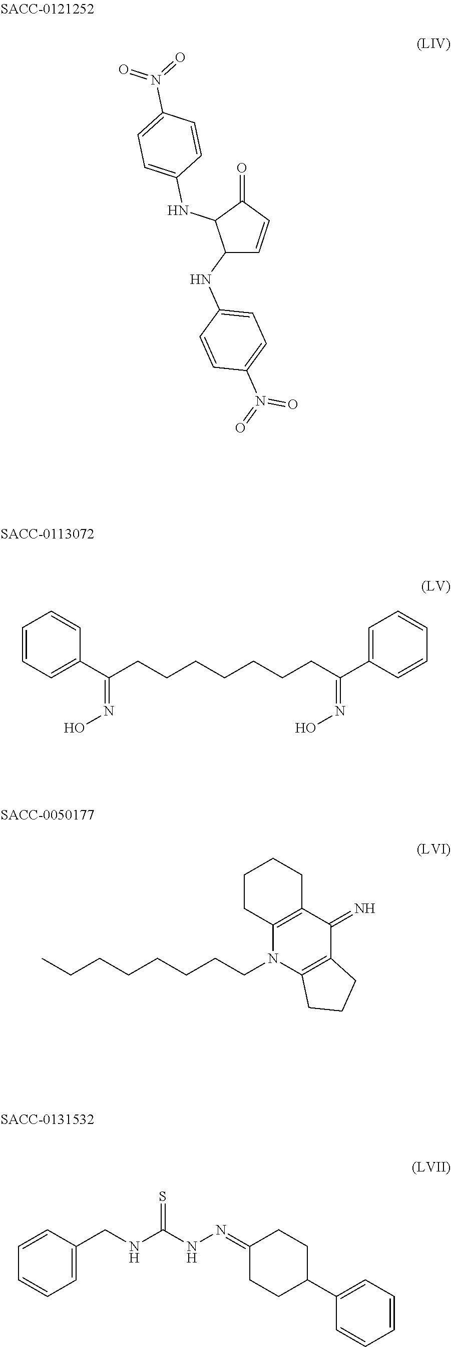 [SMILES: O=C1C=CC(Nc2ccc([N+](=O)[O-])cc2)C1Nc1ccc([N+](=O)[O-])cc1 ; O/N=C(/CCCCCCC/C(=N\O)c1ccccc1)c1ccccc1 ; CCCCCCCCn1c2c(c(=N)c3c1CCC3)CCCC2 ; S=C(NCc1ccccc1)NN=C1CCC(c2ccccc2)CC1]

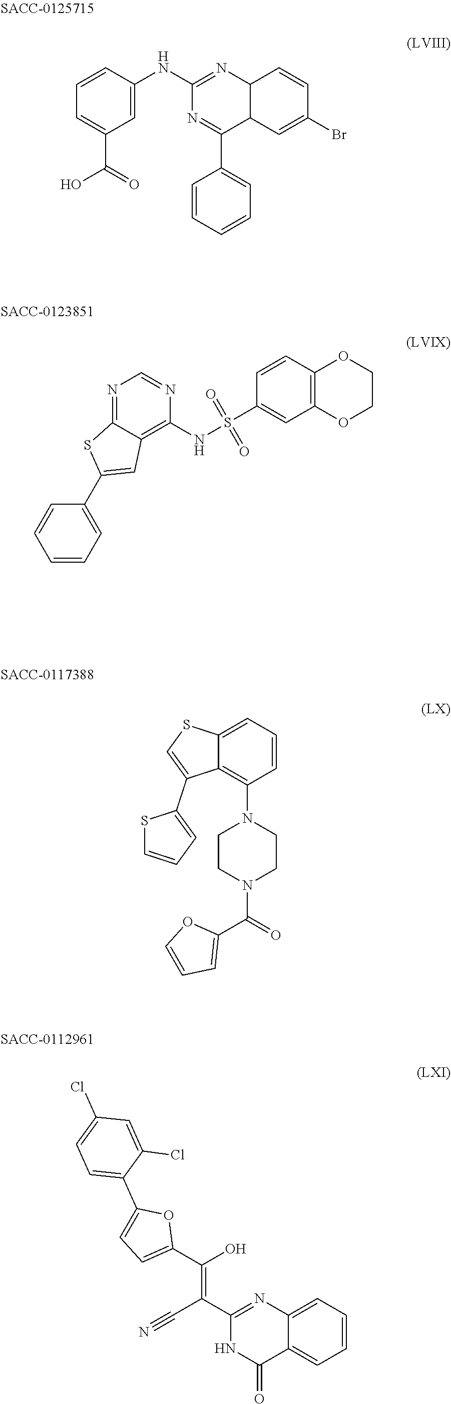 [SMILES: O=C(O)c1cccc(NC2=NC3C=CC(Br)=CC3C(c3ccccc3)=N2)c1 ; O=S(=O)(Nc1ncnc2sc(-c3ccccc3)cc12)c1ccc2c(c1)OCCO2 ; O=C(c1ccco1)N1CCN(c2cccc3scc(-c4cccs4)c23)CC1 ; N#C/C(=C(/O)c1ccc(-c2ccc(Cl)cc2Cl)o1)c1nc2ccccc2c(=O)[nH]1]

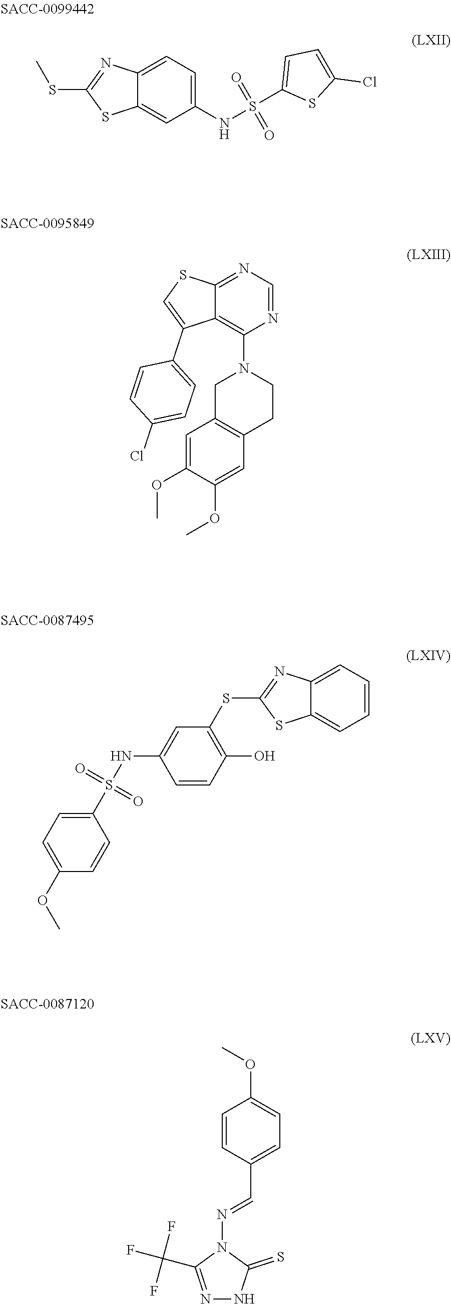 [SMILES: CSc1nc2ccc(NS(=O)(=O)c3ccc(Cl)s3)cc2s1 ; COc1cc2c(cc1OC)CN(c1ncnc3scc(-c4ccc(Cl)cc4)c13)CC2 ; COc1ccc(S(=O)(=O)Nc2ccc(O)c(Sc3nc4ccccc4s3)c2)cc1 ; COc1ccc(/C=N/n2c(C(F)(F)F)n[nH]c2=S)cc1]

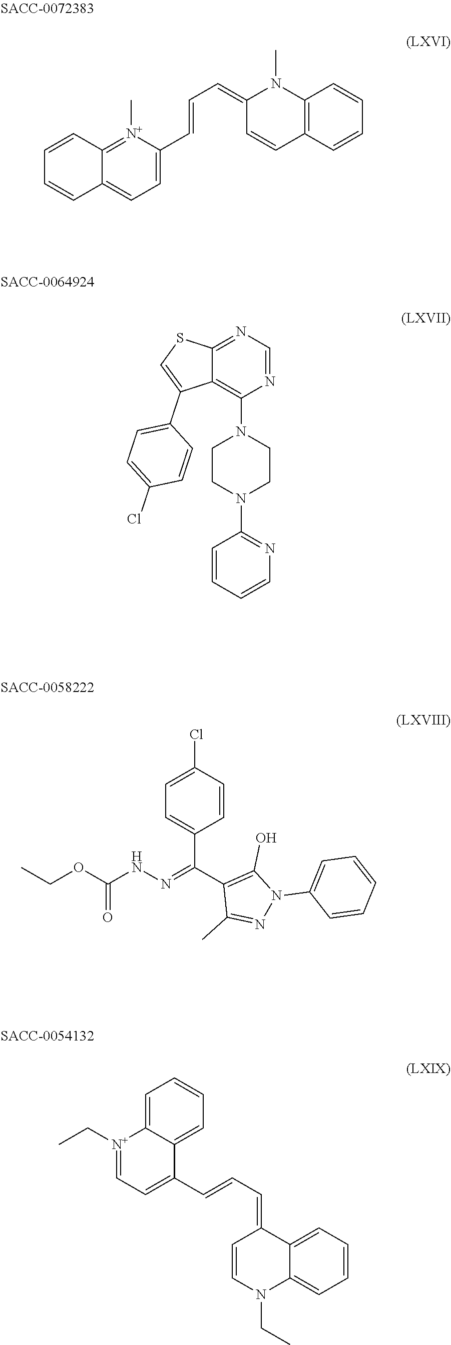 [SMILES: CN1/C(=C/C=C/c2ccc3ccccc3[n+]2C)C=Cc2ccccc21 ; Clc1ccc(-c2csc3ncnc(N4CCN(c5ccccn5)CC4)c23)cc1 ; CCOC(=O)N/N=C(\c1ccc(Cl)cc1)c1c(C)nn(-c2ccccc2)c1O ; CCN1C=C/C(=C\C=C\c2cc[n+](CC)c3ccccc23)c2ccccc21]

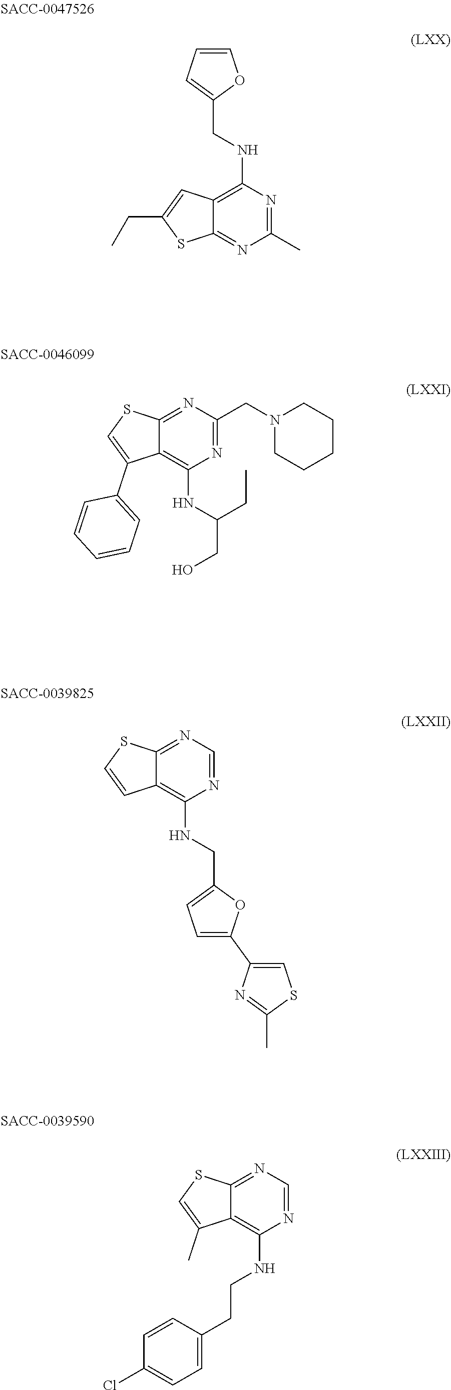 [SMILES: CCc1cc2c(NCc3ccco3)nc(C)nc2s1 ; CCC(CO)Nc1nc(CN2CCCCC2)nc2scc(-c3ccccc3)c12 ; Cc1nc(-c2ccc(CNc3ncnc4sccc34)o2)cs1 ; Cc1csc2ncnc(NCCc3ccc(Cl)cc3)c12]

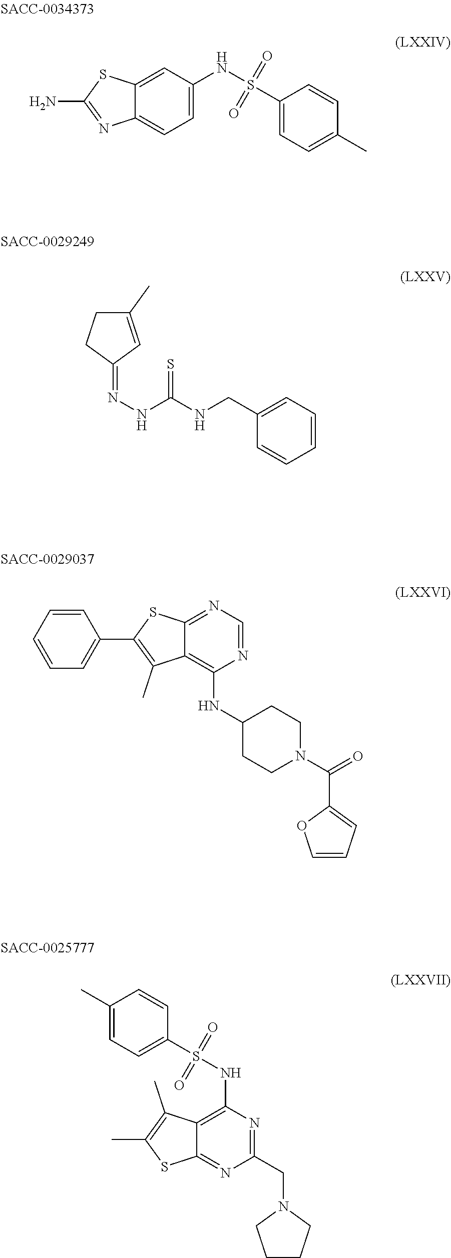

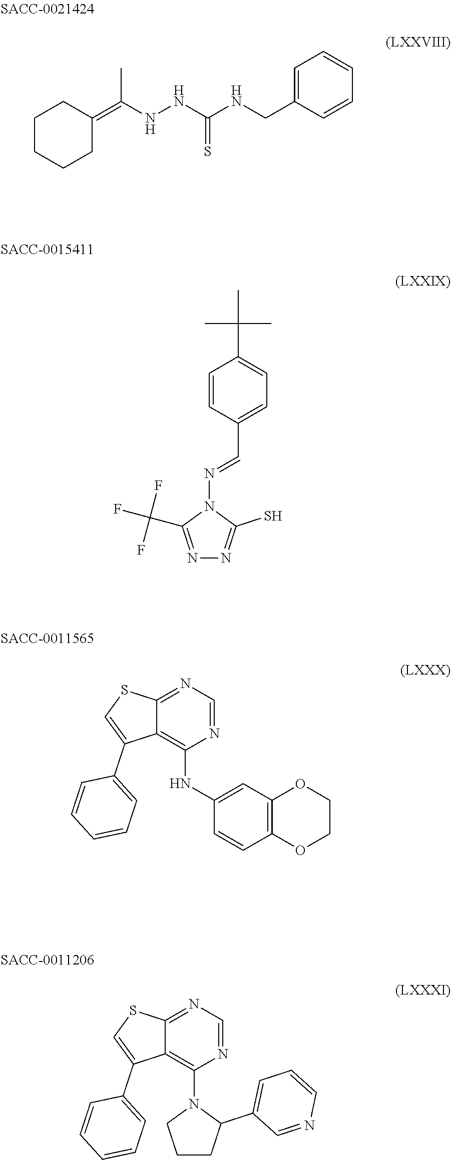 [SMILES: CC(NNC(=S)NCc1ccccc1)=C1CCCCC1 ; CC(C)(C)c1ccc(/C=N/n2c(S)nnc2C(F)(F)F)cc1 ; c1ccc(-c2csc3ncnc(Nc4ccc5c(c4)OCCO5)c23)cc1 ; c1ccc(-c2csc3ncnc(N4CCCC4c4cccnc4)c23)cc1]

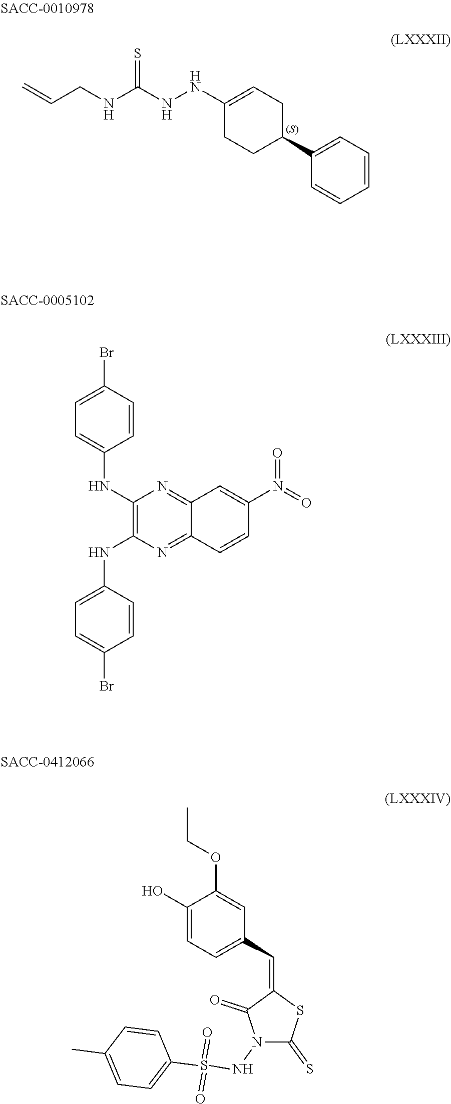

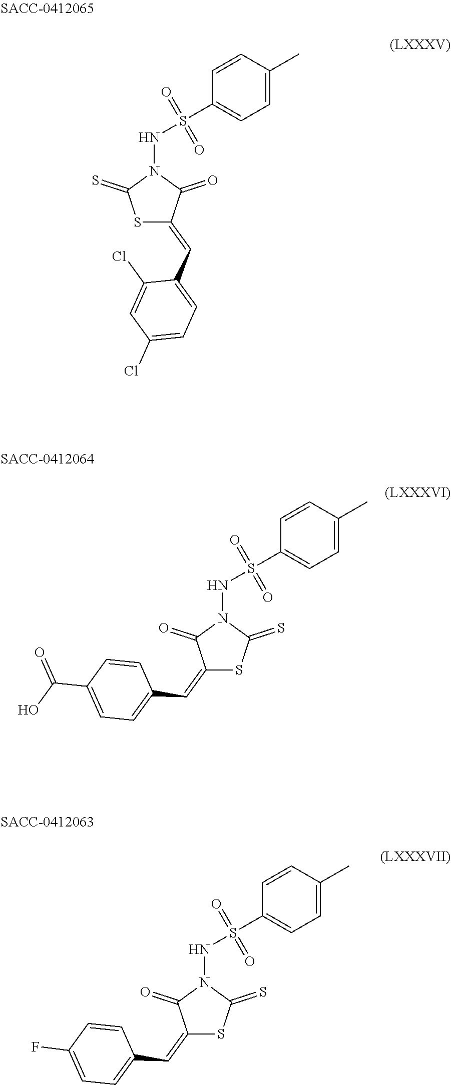

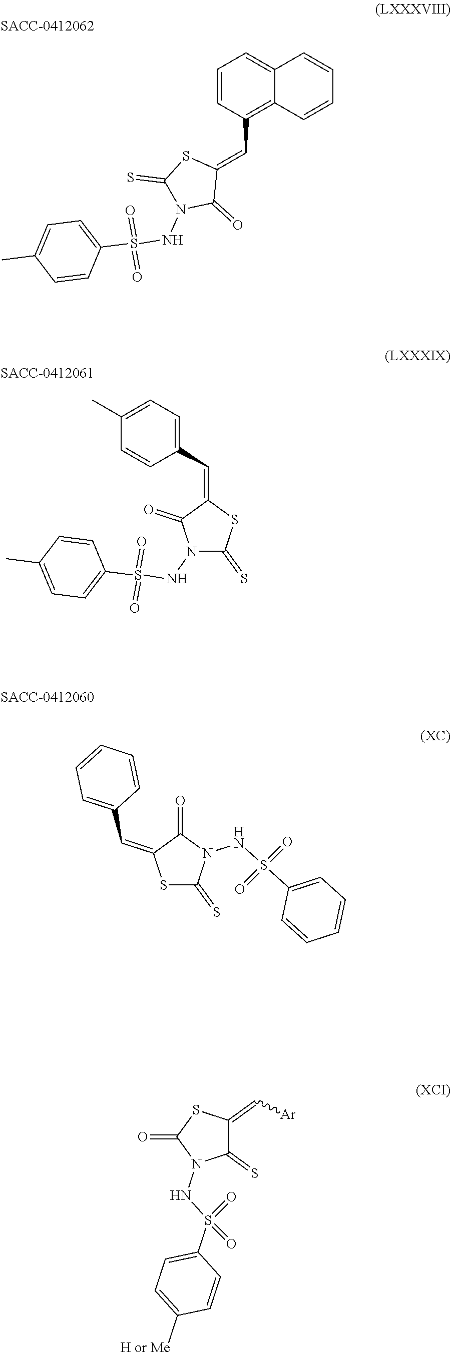

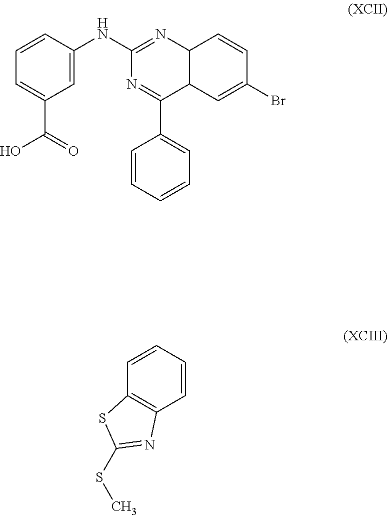 [SMILES: O=C(O)c1cccc(NC2=NC3C=CC(Br)=CC3C(c3ccccc3)=N2)c1 ; CSc1nc2ccccc2s1]

### Mixtures

The small molecules can also be used in a mixture with one or more suitable fungicides, bactericides, acaricides, molluscicides, nematicides, insecticides, microbiological agents, beneficial organisms, herbicides, fertilizers, bird repellents, phytotonics, sterilants, safeners, semiochemicals and/or plant growth regulators, in order thus, for example, to broaden the spectrum of action, prolong the period of action, enhance the rate of action, prevent repellency or prevent evolution of resistance.

In addition, the small molecules may be present in a mixture with other active ingredients or semiochemicals such as attractants and/or bird repellents and/or plant activators and/or growth regulators and/or fertilizers. Likewise, the small molecules can be used to improve plant properties, for example growth, yield and quality of the harvested material.

In a particular embodiment according to the invention, the small molecules are present in formulations or in the use forms prepared from these formulations in a mixture with further compounds, preferably those as described below.

If one of the compounds mentioned below can occur in different tautomeric forms, these forms are also included even if not explicitly mentioned in each case. All the mixing components mentioned, as the case may be, may also form salts with suitable bases or acids if they are capable of doing so on the basis of their functional groups.

### Insecticides Acaricides/Nematicides

The active ingredients specified here with their common names are known and are described for example in “The Pesticide Manual”, 16th ed., British Crop Protection Council 2012, or can be searched for on the Internet (e.g., http://www.alanwood.net/pesticides). The classification is based on the IRAC Mode of Action Classification Scheme applicable at the time of filing of this patent application.

(1) Acetylcholinesterase (AChE) inhibitors, preferably carbamates selected from alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb; or organophosphates selected from acephate, azamethiphos, azinphos-ethyl, azinphos-methyl, cadusafos, chlorethoxyfos, chlorfenvinphos, chlormephos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl) salicylate, isoxathion, malathion, mecarbam, methamidophos, methidathion, mevinphos, monocrotophos, naled, omethoate, oxydemeton-methyl, parathion-methyl, phenthoate, phorate, phosalone, phosmet, phosphamidon, phoxim, pirimiphosmethyl, profenofos, propetamphos, prothiofos, pyraclofos, pyridaphenthion, quinalphos, sulfotep, tebupirimfos, temephos, terbufos, tetrachlorvinphos, thiometon, triazophos, triclorfon and vamidothion.

(2) GABA-gated chloride channel blockers, preferably cyclodiene-organochlorines selected from chlordane and endosulfan or phenylpyrazoles (fiproles) selected from ethiprole and fipronil.

(3) Sodium channel modulators, preferably pyrethroids selected from acrinathrin, allethrin, d-cis-trans allethrin, d-trans allethrin, bifenthrin, bioallethrin, bioallethrin 5-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(IR)-trans isomer], deltamethrin, empenthrin [(EZ)-(1R) isomer], esfenvalerate, etofenprox, fenpropathrin, fenvalerate, flucythrinate, flumethrin, tau-fluvalinate, halfenprox, imiprothrin, kadethrin, momfluorothrin, permethrin, phenothrin [(1R)-trans isomer], prallethrin, pyrethrins (pyrethrum), resmethrin, silafluofen, tefluthrin, tetramethrin, tetramethrin [(IR) isomer], tralomethrin and transfluthrin or DDT or methoxychlor.

(4) Nicotinic acetylcholine receptor (nAChR) competitive modulators, preferably neonicotinoids selected from acetamiprid, clothianidin, dinotefuran, imidacloprid, nitenpyram, thiacloprid and thiamethoxam, or nicotine, or sulfoximines selected from sulfoxaflor, or butenolides selected from flupyradifurone.

(5) Nicotinic acetylcholine receptor (nAChR) allosteric modulators, preferably spinosyns selected from spinetoram and spinosad.

(6) Glutamate-gated chloride channel (GluCl) allosteric modulators, preferably avermectins/milbemycins selected from abamectin, emamectin benzoate, lepimectin and milbemectin.

(7) Juvenile hormone mimetics, preferably juvenile hormone analogues selected from hydroprene, kinoprene and methoprene, or fenoxycarb or pyriproxyfen.

(8) Miscellaneous non-specific (multisite) inhibitors, preferably alkyl halides selected from methyl bromide and other alkyl halides, or chloropicrin or sulfuryl fluoride or borax or tartar emetic or methyl isocyanate generator selected from diazomet and metam.

(9) TRPV channel modulators of chordotonal organs selected from pymetrozine and pyrifluquinazone.

(10) Mite growth inhibitors selected from clofentezine, hexythiazox, diflovidazin and etoxazole.

(11) Microbial disruptors of the insect midgut membrane selected from Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis and B.t. plant proteins selected from Cry1Ab, Cry1Ac, Crs1Fa, Cry1A, 105, Crv2Ab, Vip3A, mCry3A, Cry3Ab, Cry3Bb and Cry34 Abl/35Abl.

(12) Inhibitors of mitochondria) ATP synthase, preferably ATP disruptors selected from diafenthiuron, or organotin compounds selected from azocyclotin, cyhexatin and fenbutatin oxide or propargite or tetradifon.

(13) Uncouplers of oxidative phosphorylation via disruption of the proton gradient selected from chlorfenapyr, DNOC and sulfluramid.

(14) Nicotinic acetylcholine receptor channel blockers selected from bensultap, cartap hydrochloride, thiocyclam, and thiosultap-sodium.

(15) Inhibitors of chitin biosynthesis, type 0 selected from bistrifluron, chlorfluazuron, diflubenzuron, flucycloxuron, flufenoxuron, hexaflumuron, lufenuron, novaluron, noviflumuron, teflubenzuron and triflumuron.

(16) Inhibitors of chitin biosynthesis, type 1 selected from buprofezin.

(17) Moulting disruptors (especially in the case of Diptera) selected from cyromazine.

(18) Ecdysone receptor agonists selected from chromafenozide, halofenozide, methoxyfenozide and tebufenozide.

(19) Octopamine receptor agonists selected from amitraz.

(20) Mitochondria) complex III electron transport inhibitors selected from hydramethylnon, acequinocyl and fluacrypyrim.

(21) Mitochondria) complex I electron transport inhibitors, preferably METI acaricides selected from fenaza-quin, fenpyroximate, pyrimidifen, pyridaben, tebufenpyrad and tolfenpyrad or rotenone (Derris).

(22) Voltage-dependent sodium channel blockers selected from indoxacarb and metaflumizone.

(23) Inhibitors of acetyl CoA carboxylase, preferably tetronic and tetramic acid derivatives selected from spirodiclofen, spiromesifen and spirotetramat.

(24) Mitochondria complex IV electron transport inhibitors, preferably phosphines selected from aluminium phosphide, calcium phosphide, phosphine and zinc phosphide, or cyanides selected from calcium cyanide, potassium cyanide and sodium cyanide.

(25) Mitochondria complex II electron transport inhibitors, preferably beta-keto nitrile derivatives selected from cyenopyrafen and cyflumetofen, or carboxanilide selected from pyflubumide.

(28) Ryanodine receptor modulators, preferably diamides selected from chlorantraniliprole, cyantraniliprole and flubendiamide.

(29) Modulators of chordotonal organs (with undefined target structure) selected from flonicamide.

(30) further active ingredients selected from afidopyropen, afoxolaner, azadirachtin, benclothiaz, benzoximate, bifenazate, broflanilide, bromopropylate, chinomethionat, chloroprallethrin, cryolite, cyclaniliprole, cycloxaprid, cyhalodiamide, dicloromezotiaz dicofol, epsilon metofluthrin, epsilon momfluthrin, flometoq Puin, fluazaindolizine, fluensulfone, flufenerim, flufenoxystrobin, flufiprole, fluhexafon, fluopyram, fluralaner, fluxametamide, fufenozide, guadipyr, heptafluthrin, imidaclothiz, iprodione, kappa bifenthrin, kappa tefluthrin, lotilaner, meperfluthrin, paichongding, pyridalyl, pyrifluq Puinazon, pyriminostrobin, spirobudiclofen, tetramethylfluthrin, tetraniliprole, tetrachlorantraniliprole, tigolaner, tioxazafen, thiofluoximate, triflumezopyrim and iodomethane; additionally preparations based on Bacillus firmus (I-1582, BioNeem, Votivo), and the following compounds: 1-(2-fluoro-4-methyl-5-[(2,2,2-trifluoroethyl)sulfinyl]phenyl )-3-(trifluoromethyl)-1H-1,2,4-triazole-5-amine (known from WO2006/043635) (CAS 885026-50-6), (1′-[(2E)-3-(4-chlorophenyl)prop-2-en-1-yl]-5-fluorospiro [indole-3,4′-piperidine]-1 (2H)-yl)(2-chloropyridin-4-yl)methanone (known from WO2003/106457) (CAS 637360-23-7), 2-chloro-N-[2-(1-[(2E)-3-(4-chlorophenyl 1)prop-2-en-1-yl]piperidin-4-yl)-4-(trifluoromethyl)phenyl] isonicotinamide (known from WO2006/003494) (CAS 872999-66-1), 3-(4-chloro-2,6-dimethylphenyl)-4-hydroxy-8-methoxy-1,8-diazaspiro[4.5]dec-3-en-2-one (known from WO 2010052161) (CAS 1225292-17-0), 3-(4-chloro-2,6-dimethylphenyl)-8-methoxy-2-oxo-1,8-diazaspiro[4.5]dec-3-en-4-yl ethylcarbonate (known from EP 2647626) (CAS 144051642-6), 4-(but-2-yn-1-yloxy)-6-(3,5-dimethylpiperidin-1-yl)-5-fluoropyrimidine (known from WO2004/099160) (CAS 792914-58-0), PF1364 (known from JP2010/018586) (CAS 1204776-60-2), N-[(2E)-1-[(6-chloropyridin-3-yl)methyl]pyridin-2(1H)-ylidene]-2,2,2-trifluoroacetamide (known from WO2012/029672) (CAS 1363400-41-2), (3E)-3-[1-[(6-chloro-3-pyridyl)methyl]-2-pyridylidene]-1,1,1-trifluoropropan-2-one (known from WO2013/144213) (CAS 1461743-15-6), N-[3-(benzylcarbamoyl)-4-chlorophenyl]l-methyl-3-(pentafluoroethyl)-4-(trifluoromethyl)-1H-pyrazole-5-carboxamide (known from WO2010/051926) (CAS 1226889-14-0), 5-bromo-4-chloro-N-[4-chloro-2-methyl-6-(methylcarbamoyl)phenyl]-2-(3-chloro-2-pyridyl)pyrazole 3-carboxamide (known from CN103232431) (CAS 1449220-44-3), 4-[5-(3,5-dichlorophenyl)-4,5-dihydro-5-(trifluoromethyl)-3-isoxazolyl]-2-methyl-N-(cis-1-oxido-3-thietanyl)benzamide, 4-[5-(3,5-dichlorophenyl)-4,5-dihydro-5-(trifluoromethyl)-3-isoxazolyl]-2-methyl-N-(transl-oxido-3-thietanyl)benzamide and 4-[(5S)-5-(3,5-dichlorophenyl)-4, 5-dihydro-5-(trifluoromethyl)-3-isoxazoly 1]-2-methyl-N-(cis-1-oxido-3-thietanyl)benzamide (known from WO 2013/050317 A1) (CAS 1332628-83-7). N-[3-chloro-1-(3-pyridinyl)-1H-pyrazol-4-yl]-N-ethyl-3-[(3,3,3-trifluoropropyl)sulfinyl]propanamide, (+)—N-[3-chloro-1-(3-pyridinyl)-1H-pyrazol-4-yl]-N-ethyl-3-[(3,3,3-trifluoropropyl)sulfinyl]propanamide and (−)—N-[3-chloro-1-(3-pyridinyl)-1H-pyrazol-4-yl]-N-ethyl-3-[(3,3,3-trifluoropropyl)sulfinyl]propanamide (known from WO2013/162715 A2, WO 2013/162716 A2, US 2014/0213448 A1) (CAS 1477923-37-7), 5-[[(2E)-3-chloro-2-propen-1-yl]amino]-1-[2,6-dichloro-4-(trifluoromethyl)phenyl]-4-[(trifluoromethyl)sulfinyl]-1H-pyrazole-3-carbonitrile (known from CN 101337937 A) (CAS 1105672-77-2), 3-bromo-N[4-chloro-2-methyl-6-[(methylamino)thioxomethyl]phenyl]-1-(3-chloro-2-pyridinyl)-1H-pyrazole-5-carboxamide, (Liudaibenjiaxuanan, known from CN 103109816 A) (CAS 1232543-85-9); N-[4-chloro-2-[[(1,1-dimethylethyl)amino]carbonyl]-6-methylphenyl]-1-(3-chloro-2-pyridinyl)-3-(fluoromethoxy)-1H-pyrazole-5-carboxamide (known from WO 2012/034403 A1) (CAS 1268277-22-0). N-[2-(5-amino-1,3,4-thiadiazol-2-yl)-4-chloro-6-methylphenyl]-3-bromo-1-(3-chloro-2-pyridinyl)-1H-pyrazole-5-carboxamide (known from WO 2011/085575 A1) (CAS 1233882-22-8), 4-[3-[2,6-dichloro-4-[(3,3-dichloro-2-propen-1-yl)oxy]phenoxy]propoxy]-2-methoxy-6-(trifluoromethyl)pyrimidine (known from CN 101337940 A) (CAS 1108184-52-6); (2E)- and 2(Z)-2-[2-(4-cyanophenyl)-1-[3-(trifluoromethyl)phenyl]ethylidene]-N-[4-(difluoromethoxy)phenyl]hydrazinecarboxamide (known from CN 101715774 A) (CAS 1232543-85-9); cyclopropanecarboxylic acid 3-(2,2-dichloroethenyl)-2,2-dimethyl-4-(1H-benzimidazol-2-yl)phenyl ester (known from CN 103524422 A) (CAS 1542271-46-4); (4aS)-7-chloro-2,5-dihydro-2-[[(methoxycarbonyl) [4 [(trifluoromethyl)thio] phenyl] amino] carbonyl] indeno [1,2-e][1,3,4]oxadiazine-4a(3H)-carboxylic acid methyl ester (known from CN 102391261 A) (CAS 1370358-69-2); 6-deoxy-3-O-ethyl-2,4-di-O-methyl-1-[N-[4-[1-[4-(1,1,2,2,2-pentafluoroethoxy)phenyl]-1H-1,2,4-triazole-3-yl]phenyl]carbamate]-a-L-mannopyranose (known from US 2014/0275503 A1) (CAS 1181213-14-8); 8-(2-cyclopropylmethoxy-4-trifluoromethylphenoxy)-3-(6-trifluoromethylpyridazin-3-yl)-3-azabicyclo[3.2.1]octane (CAS 1253850-56-4). (8-anti)-8-(2-cyclopropylmethoxy-4-trifluoromethylphenoxy)-3-(6-trifluoromethylpyridazin-3-yl)-3-azabicyclo[3.2.1]octane (CAS 933798-27-7). (8-syn)-8-(2-cyclopropylmethoxy-4-trifluoromethylphenoxy)-3-(6-trifluoromethylpyridazin-3-yl)-3-azabicyclo[3.2.1]octane (known from WO 2007(40280 A1, WO 2007040282 A1) (CAS 934001-66-8), N-[3-chloro-1-(3-pyridinyl)-1H-pyrazol-4-yl]-N-ethyl-3-[(3,3,3-trifluoropropyl)thio]propanamide (known from WO 2015/058021 A1, WO 2015/058028 A1) (CAS 1477919-27-9) and N-[4(aminothioxomethyl)-2-methyl-6-[(methylamino)carbonyl]phenyl]-3-bromo-1-(3-chloro-2-pyridinyl)1H-pyrazole-5-carboxamide (known from CN 103265527A) (CAS 1452877-50-7), 5-(1,3-dioxan-2-yl)-4-[[4-(trifluoromethyl) phenyl]methoxy]pyrimidine (known from WO 2013/115391 A1) (CAS 1449021-97-9), 3-(4-chloro-2,6-dimethylphenyl)-4-hydroxy-8-methoxy-1-methyl-1,8-diazaspiro[4.5]dec-3-en-2-one (known from WO 2010/066780 A1, WO 2011/151140 A1) (CAS 1229023-34-0), 3-(4-chloro-2,6-dimethylphenyl)-8-methoxy-1-methyl-1,8-diazaspiro[4.5]decane-2,4-dione (known from WO 2014/187846 A1) (CAS 1638765-58-8), 3-(4-chloro-2,6-dimethylphenyl)-8-methoxy-1-methyl-2-oxo-1,8-diazaspiro[4.5]dec-3-en-4-ylcarboxylic acid ethyl ester (known from WO 2010/006780 A1, WO 2011151146 A1) (CAS 1229023-00-0). N-[1-[(6-chloro-3-pyridinyl)methyl]-2(1H)-pyridinylidene]-2,2,2-trifluoroacetamide (known from DE 3639877 A1, WO 2012029672 A1) (CAS 1363400-41-2), [N(E)]—N-[1-[(6-chloro-3-pyridinyl)methyl]-2(1H)-pyridinylidene]-2,2,2-trifluoroacetamide (known from WO 2016005276 A1) (CAS 1689566-03-7), [N(Z)]-N-[1-[(6-chloro-3-pyridinyl)methyl]-2(1H)-pyridinylidene]-2,2,2-trifluoroacetamide (CAS 1702305-40-5), 3-endo-3-[2-propoxy-4-(trifluoromethyl)phenoxy]-9-[[5-(trifluoromethyl)-2-pyridinyl]oxy]-9-azabicyclo[3.3.1]nonane (known from WO 2011/105506 A1, WO 2016/133011 A1) (CAS 1332838-17-1).

### Fungicides

The active ingredients specified herein by their common name are known and described, for example, in “Pesticide Manual” (16th Ed. British Crop Protection Council) or searchable on the internet (for example: http://www.alanwood.net/pesticides).

All the mixing components mentioned in classes (1) to (15), as the case may be, may form salts with suitable bases or acids if they are capable of doing so on the basis of their functional groups. All the fungicidal mixing components mentioned in classes (1) to (15), as the case may be, may include tautomeric forms.

1) Ergosterol biosynthesis inhibitors, for example (1.001) cyproconazole, (1.002) difenoconazole, (1.003) epoxiconazole, (1.004) fenhexamid, (1.005) fenpropidin, (1.006) fenpropimorph, (1.007) fenpyrazamine, (1.008) fluquinconazole. (1.009) flutriafol, (1.010) imazalil, (1.011) imazalil sulfate, (1.012) ipconazole, (1.013) metconazole, (1.014) myclobutanil, (1.015) paclobutrazol, (1.016) prochloraz, (1.017) propiconazole, (1.018) prothioconazole, (1.019) pyrisoxazole, (1.020) spiroxamine, (1.021) tebuconazole. (1.022) tetraconazole, (1.023) triadimenol, (1.024) tridemorph, (1.025) triticonazole. (1.026) (1R,2S,5S)-5-(4-chlorobenzyl)-2-(chloromethyl)-2-methyl(1H-1,2,4-triazol-1-ylmethyl)cyclopentanol, (1.027) (1S,2R,5R)-5-(4-chlorobenzyl)-2-(chloromethyl)-2-methyl-1-(1H-1,2,4-triazol-1-ylmethyl)cyclopentanol, (1.028) (2R)-2-(1-chlorocyclopropyl)-4-[(1R)-2,2-dichlorocyclopropyl]-(1H-1,2,4-triazol-1-yl)butan-2-ol (1.029) (2R)-2-(1-chlorocyclopropyl)-4-[(1 S)-2,2-dichlorocyclopropyl]-1-(1H-1,2,4-triazol-1-yl)butan-2-ol, (1.030) (2R)-2-[4-(4-chlorophenoxy)-2-(trifluoromethyl)phenyl]-1-(1H-1,2,4-triazol-1-yl)propan-2-ol, (1.031) (2S)-2-(1-chlorocyclopropyl)-4-[(1R)-2,2-dichlorocyclopropyl]-1-(1H-1,2,4-triazol-1-yl)butan-2-ol, (1.032) (2S)-2-(1-chlorocyclopropyl)-4-[(1 S)-2,2-dichlorocyclopropyl]-1-(1H-1,2,4-triazol-1-yl)butan-2-ol, (1.033) (2S)-2-[4-(4-chlorophenoxy)-2-(trifluoromethyl)phenyl]-1-(1H-1,2,4-triazol-1-yl)propan-2-ol, (1.034) (R)-[3-(4-chloro-2-fluorophenyl)-5-(2,4-difluorophenyl)-1,2-oxazol-4-yl](pyridin-3-yl)methanol, (1.035) (S)-[3-(4-chloro-2-fluorophenyl)-5-(2,4-difluorophenyl)-1,2-oxazol-4-yl] (pyridin-3-yl)methanol, (1.036) [3-(4-chloro-2-fluorophenyl)-5-(2,4-difluorophenyl)-1,2-oxazol-4-yl](pyridin-3-yl)methanol, (1.037) 1-({(2R,4S)-2-[2-chloro-4-(4-chlorophenoxy)phenyl]-4-methyl-1,3-dioxolan-2-yl}methyl)-1H-1,2,4-triazole, (1.038) 1-({(2S,4S)-2-[2-chloro-4-(4-chlorophenoxy)phenyl]-4-methyl-1,3-dioxolan-2-yl}methyl)-1H-1,2,4-triazole, (1.039) 1-{[3-(2-chlorophenyl)-2-(2,4-difluorophenyl)oxiran-2-yl]methyl}1H-1,2,4-triazol-5-yl thiocyanate, (1.040)1-{[rel(2R,3R)-3-(2-chlorophenyl)-2-(2,4-difluorophenyl)oxiran-2-yl]methyl}-1H-1,2,4-triazol-5-yl thiocyanate, (1.041) 1-{[rel(2R,3S)-3-(2-chlorophenyl)-2-2,4-difluorophenyl)oxiran-2-yl]methyl}-1H-1,2,4-triazol-5-yl thiocyanate, (1.042) 2-[(2R,4R,5R)-1-(2,4-dichlorophenyl)-5-hydroxy-2,6,6-trimethylheptan-4-yl]-2,4-dihydro-3H-1,2,4-triazole-3-thione, (1.043) 2-[(2R,4R,5S)-1-(2,4-dichlorophenyl)-5-hydroxy-2,6,6-trimethylheptan-4-yl]-2,4-dihydro-3H-1,2,4-triazole-3-thione, (1.044) 2-[(2R,4S,5R)-1-(2,4-dichlorophenyl)-5-hydroxy-2,6,6-trimethylheptan-4-yl]-2,4-dihydro-3H-1,2,4-triazole-3-thione, (1.045) 2-[(2R,4S,5S)-1-(2,4dichlorophenyl)-5-hydroxy-2,6,6-trimethylheptan-4-yl]-2,4-dihydro-3H-1,2,4-triazole-3-thione, (1.046) 2-[(2S,4R,5R)-1-(2,4-dichlorophenyl)-5-hydroxy-2,6,6-trimethylheptan-4-yl]-2,4-dihydro-3H-1,2,4-triazole-3-thione, (1.047) 2-[(2S,4R,5S)-1-(2,4-dichlorophenyl)-5-hydroxy-2,6,6-trimethylheptan-4-yl]-2,4-dihydro-3H-1,2,4-triazole-3-thione, (1.048) 2-[(2S,4S,5R)-1-(2,4-dichlorophenyl)-5-hydroxy-2,6,6-trimethylheptan-4-yl]-2,4-dihydro-3H-1,2,4-triazole-3-thione, (1.049) 2-[(2S,4S,5S)-1-(2,4-dichlorophenyl)-5-hydroxy-2,6,6-trimethylheptan-4-yl]-2,4-dihydro-3H-1,2,4-triazole-3-thione, (1.050) 2-[1-(2,4-dichlorophenyl)-5-hydroxy-2,6,6-trimethylheptan-4-yl]-2,4-dihydro-3H-1,2,4-triazole-3-thione, (1.051) 2-[2-chloro-4-(2,4-dichlorophenoxy)phenyl]-1-(1H-1,2,4-triazol-1-yl)propan-2-ol, (1.052) 2-[2-chloro-4-(4-chlorophenoxy)phenyl]-1-(1H-1,2,4-triazol-1-yl)butan-2-ol, (1.053) 2-[4(4-chlorophenyl)-2-(2,4-difluorophenyl)oxiran-2-yl]methyl)-2,4-dihydro-3H-1,2,4-triazole-3-thione, (1.057) 2-{[rel(2R,3R)-3-(2-chlorophenyl)-2-(2,4-difluorophenyl)oxiran-2-yl]methyl}-2,4-dihydro-3H-1,2,4-triazole-3-thione, (1.058) 2-{[rel(2R,3S)-3-(2-chlorophenyl)-2-(2,4-difluorophenyl) oxiran-2-yl]methyl}-2,4-dihydro-3H-1,2,4-triazole-3-thione, (1.059) 5-(4-chlorobenzyl)-2-(chloromethyl)-2-methyl-1-(1H-1,2,4-triazol-1-ylmethyl)cyclopentanol, (1.060) 5-(allylsulfanyl)-1-[[3-(2-chlorophenyl)-2-(2,4-difluorophenyl)oxiran-2-yl]methyl)-1H-1,2,4-triazole, (1.061) 5-(allylsulfanyl)-1-{[rel(2R,3R)-3-(2-chlorophenyl)-2-(2,4-difluorophenyl)oxiran-2-yl]methyl}-1H-1,2,4-triazole, (1.062) 5-(allylsulfanyl)-chlorophenoxy)-2-(trifluoromethyl)phenyl]-1-(1H-1,2,4-triazol-1-yl)butan-2-ol, (1.054) 2-[4-(4-chlorophenoxy)-2-(trifluoromethyl)phenyl]-1-(1H-1,2,4-triazol-1-yl)pentan-2-ol, (1.055) 2-[4-(4chlorophenoxy)-2-(trifluoromethyl)phenyl]-1-(1H-1,2,4-triazol-1-vi) propan-2-ol, (1.056) 2-[3-(2-chlorophenyl)-2-(2,4-difluorophenyl)oxiran-2-yl]methyl)-2,4-dihydro-3H-1,2,4-triazole-3-thione, (1.057) 2-{[rel(2R,3R)-3-(2-chlorophenyl)-2-(2,4-difluorophenyl)oxiran-2-yl]methyl}-2,4-dihydro-3H-1,2,4-triazole-3-thione, (1.058) 2-{[rel(2R,3S)-3-(2-chlorophenyl)-2-(2,4-difluorophenyl)oxiran-2-yl]methyl}-2,4-dihydro-3H-1,2,4-triazole-3-thione, (1.059) 5-(4-chlorobenzyl)-2-(chloromethyl)-2-methyl-1-(1H-1,2,4-triazol-1-ylmethyl)cyclopentanol, (1.060) 5-(allylsulfanyl)-1-{[3-(2-chlorophenyl)-2-(2,4-difluorophenyl)oxiran-2-yl]methyl}-1H-1,2,4-triazole, (1.061) 5-(alkylsulfanyl)-1-[rel(2R,3R)-3-(2-chlorophenyl)-2-(2,4-difluorophenyl)oxiran-2-yl]methyl-1H-1,2,4-triazole, (1.062) 5-(allylsulfanyl)-1-([rel(2R,3S)-3-(2-chlorophenyl)-2-(2,4-difluorophenyl)oxiran-2-yl]methyl)-1H-1,2,4-triazole, (1.063) N′-(2,5-dimethyl-4-([3-(1,1,2,2-tetrafluoroethoxy)phenyl]sulfanyl)phenyl)-N-ethyl-N-methylimidoformamide, (1.064) N′-(2,5-dimethyl-4-([3-(2,2,2-trifluoroethoxy)phenyl]sulfanyl)phenyl)-N-ethyl-Nmethylimidoformamide, (1.065) N′-(2,5-dimethyl-4-([3-(2,2,3,3-tetrafluoropropoxy)phenyl] sulfanyl)phenyl)-N-ethyl-N-methylimidoformamide, (1.066) N′-(2,5-dimethyl-4-([3-(pentafluoroethoxy)phenyl]sulfanyl)phenyl)-N-ethyl-Nmethylimidoformamide, (1.067) N′-(2,5-dimethyl-4-(3-[(1,1,2,2-tetrafluoroethyl)sulfanyl]phenoxy)phenyl)-N-ethyl-N-methylimidoformamide, (1.068) N′-(2,5-dimethyl-4-(3-[(2,2,2-trifluoroethyl)sulfanyl]phenoxy)phenyl)-N-ethyl-N-methylimidoformamide, (1.069) N′-(2,5-dimethyl-4-{3-[(2,2,3,3-tetrafluoropropyl)sulfanyl]phenoxy}phenyl)-N-ethyl-N-methylimidoformamide, (1.070) N′-(2,5-dimethyl-4-(3-[(pentafluoroethyl)sulfanyl]phenoxy)phenyl)-N-ethyl-Nmethylimidoformamide, (1.071) N′-(2,5-dimethyl-4-phenoxyphenyl)-N-ethyl-N-methylimidoformamide, (1.072) N-(4-([3-(difluoromethoxy)phenyl]sulfanyl)-2,5-dimethylphenyl)-N-ethyl-N-methylimidoformamide, (1.073) N′-(4-(3-[(difluoromethyl)sulfanyl]phenoxy)-2,5-dimethylphenyl)-N-ethyl-N-methylimidoformamide, (1.074) N-[5-bromo-6-(2,3-dihydro-1H-inden-2-yloxy)-2-methylpyridin-3-yl]-N-ethyl-N-methylimidoformamide, (1.075) N′-(4-[(4,5-dichloro-1,3-thiazol-2-yl)oxy]-2,5-dimethylphenyl)-Nethyl-N-methylimidoformamide, (1.076) N′-(5-bromo-6-[(1R)-1-(3,5-difluorophenyl)ethoxy]-2-methylpyridin-3-yl)-N-ethyl-N-methylimidoformamide, (1.077) N-(5-bromo-6-[(1 S)-1-(3,5-difluorophenyl)ethoxy]-2-methylpyridin-3-yl)-N-ethyl-N-methylimidoformamide, (1.078) N′-{5-bromo-6-[(cis-4-isopropylcyclohexyl)oxy]-2-methylpyridin-3-yl}-N-ethyl-N-methylimidoformamide, (1.079) N-(5-bromo-6-[(trans-4-isopropylcyclohexyl)oxy]-2-methylpyridin-3-yl)-N-ethyl-N-methylimidoformamide, (1.080) N′-(5-bromo-6-[1-(3,5-difluorophenyl)ethoxy]-2-methylpyridin-3-yl)-N-ethyl-N-methylimidoformamide, (1.081) mefentrifluconazole, (1.082) ipfentrifluconazole.

2) Inhibitors of the respiratory chain in complex I or II, for example (2.001) benzovindiflupyr, (2.002) bixafen, (2.003) boscalid, (2.004) carboxin, (2.005) fluopyram, (2.006) flutolanil, (2.007) fluxapyroxad, (2.008) furametpyr, (2.009) isofetamid, (2.010) isopyrazam (anti-epimeric enantiomer 1R,4S,9S), (2.011) isopyrazam (anti-epimeric enantiomer 1S,4R,9R), (2.012) isopyrazam (anti-epimeric racemate 1RS,4SR,9SR), (2.013) isopyrazam (mixture of the syn-epimeric racemate 1RS,4SR,9RS and the anti-epimeric racemate 1RS,4SR,9SR), (2.014) isopyrazam (syn-epimeric enantiomer 1R,4S,9R), (2.015) isopyrazam (syn-epimeric enantiomer 1S,4R,9S), (2.016) isopyrazam (syn-epimeric racemate 1RS,4SR,9RS), (2.017) penflufen. (2.018) penthiopyrad, (2.019) pydiflumetofen, (2.020) pyraziflumid, (2.021) sedaxane, (2.022) 1,3-dimethyl-N-(1,1,3-trimethyl-2,3-dihydro-1H-inden-4-yl)-1H-pyrazole-4-carboxamide, (2.023) 1,3-dimethyl-N-[(3R)-1,1,3-trimethyl-2,3-dihydrol 1H-inden-4-yl]-1H-pyrazole-4-carboxamide, (2.024) 1,3-dimethyl-N-[(3S)-1,1,3-trimethyl-2,3-dihydro-1H-inden-4-yl]-1H-pyrazole-4-carboxamide, (2.025) I-methyl-3-(trifluoromethyl)-N-[2′-(trifluoromethyl)biphenyl-2-yl]-1H-pyrazole-4-carboxamide, (2.026) 2-fluoro-6-(trifluoromethyl)-N-(1,1,3-trimethyl-2,3-dihydro-1H-inden-4-yl)benzamide, (2.027) 3-(difluoromethyl)-1-methyl-N-(1,1,3-trimethyl-2,3-dihydro-1H-inden-4-yl)-1H-pyrazole-4-carboxamide, (2.028) 3-(difluoromethyl)-1-methyl-N-[(3R)-1,1,3-trimethyl-2,3-dihydro-1H-inden-4-yl]-1H-pyrazole-4-carboxamide, (2.029) 3-(difluoromethyl)-1-methyl-N-[(3S)-1,1,3-trimethyl-2,3-dihydro-1H-inden-4-yl]-1H-pyrazole-4-carboxamide, (2.030) 3-(difluoromethyl)-N-(7-fluoro-1,1,3-trimethyl-2,3-dihydro-1H-inden-4-yl)-1-methyl-1H-pyrazole-4-carboxamide. (2.031) 3-(difluoromethyl)-N-[(3R)-7-fluoro-1,1,3-trimethyl-2,3-dihydro-1H-inden-4-yl]-1-methyl-1H-pyrazole-4-carboxamide, (2.032) 3-(difluoromethyl)-N[(3S)-7-fluoro-1,1,3-trimethyl-2,3-dihydro-1H-inden-4-yl]1-methyl-1H-pyrazole-4-carboxamide, (2.033) 5,8-difluoroN-[2-(2-fluoro-4-{[4-(trifluoromethyl)pyridin-2-yl]oxy}phenyl)ethyl]quinazolin-4-amine, (2.034) N-(2-cyclopentyl-5-fluoro-1-benzyl)-N-cyclopropyl-3-(difluoromethyl)-5-fluoro-1-methyl-1H-pyrazole-4-carboxamide, (2.035) N-(2-tat-butyl-5-methylbenzyl)-Ncyclopropyl-3-(difluoromethyl)-5-fluoro-1-methyl-1H-pyrazole-4-carboxamide. (2.036) N-(2-tert-butylbenzyl)-Ncyclopropyl-3-(difluoromethyl)-5-fluoro-1-methyl-1H-pyrazole-4-carboxamide, (2.037) N-(5-chloro-2-ethylbenzyl)-N-cyclopropyl-3-(difluoromethyl)-5-fluoro-1-methyl-1H-pyrazole-4-carboxamide, (2.038) N-(5-chloro-2-isopropylbenzyl)-N-cyclopropyl-3-(difluoromethyl)-5-fluoro-1-methyl-1H-pyrazole-4-carboxamide, (2.039) N-[(1R4S)-9-(dichloromethylene)-1,2,3,4-tetrahydro-1,4-methanonaphthalen-5-yl]-3-(difluoromethyl)-1-methyl-1H-pyrazole-4-carboxamide, (2.040) N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide, (2.041) N-[1-(2,4-dichlorophenyl)-1-methoxypropan-2-yl]-3-(difluoromethyl)-1-methyl-1H-pyrazole-4-carboxamide, (2.042) N-[2-chloro-6-(trifluoromethyl)benzyl]-N-cyclopropyl-3-(difluoromethyl)-5-fluoro-1-methyl-1H-pyrazole-4-carboxamide, (2.043) N-[3-chloro-2-fluoro-6-(trifluoromethyl)benzyl]—N-cyclopropyl-3-(difluoromethyl)-5-fluorol-methyl-1H-pyrazole-4-carboxamide, (2.044) N-[5-chloro-2-(trifluoromethyl)benzyl]-N-cyclopropyl-3-(difluoromethyl)-5-fluoro-1-methyl-1H-pyrazole-4-carboxamide, (2.045) N-cyclopropyl-3-(difluoromethyl)-5-fluoro-1-methyl-N-[5-methyl-2-(trifluoromethyl)benzyl]-1H-pyrazole-4-carboxamide, (2.046) N-cyclopropyl-3-(difluoromethyl)-5-fluoro-N-(2-fluoro-6-isopropylbenzyl)-1-methyl-1H-pyrazole-4-carboxamide, (2.047) N-cyclopropyl-3-(difluoromethyl)-5-fluoro-N-(2-isopropyl-5-methylbenzyl)-1-methyl-1H-pyrazole-4-carboxamide, (2.048) N-cyclopropyl-3-(difluoromethyl)-5-fluoro-N-(2-isopropylbenzyl)-1-methyl-1H-pyrazole-4-carbothioamide, (2.049) N-cyclopropyl-3-(difluoromethyl)-5-fluoro-N-(2-isopropylbenzyl)-1-methyl-1H-pyrazole-4-carboxamide, (2.050) N-cyclopropyl-3-(difluoromethyl)-5-fluoro-N-(5-fluoro-2-isopropylbenzyl)-1-methyl-1H-pyrazole-4-carboxamide, (2.051) N-cyclopropyl-3-(difluoromethyl)-N-(2-ethyl-4,5-dimethylbenzyl)-5-fluoro-1-methyl-1H-pyrazole-4-carboxamide, (2.052) N-cyclopropyl-3-(difluoromethyl)N-(2-ethyl-5-fluorobenzyl)-5-fluoro-1-methyl-1Hpyrazole-4-carboxamide, (2.053) N-cyclopropyl-3-(difluoromethyl)-N-(2-ethyl-5-methylbenzyl)-5-fluoro-lmethyl-1H-pyrazole-4-carboxamide, (2.054) N-cyclopropyl-N-(2-cyclopropyl-5-fluoro benzyl)-3-(difluoromethyl)-5-fluoro-1-methyl-1H-pyrazole-4-carboxamide, (2.055) N-cyclopropyl-N-(2-cyclopropyl-5-methylbenzyl)-3-(difluoromethyl)-5-fluoro-1-methyl-1H-pyrazole-4-carboxamide, (2.056) N-cyclopropyl-N-(2-cyclopropylbenzyl)-3-(difluoromethyl)-5-fluoro-1-methyl-1H-pyrazole-4-carboxamide.

3) Inhibitors of the respiratory chain in complex III, for example (3.001) ametoctradin, (3.002) amisulbrom, (3.003) azoxystrobin, (3.004) coumethoxystrobin, (3.005) coumoxystrobin, (3.006) cyazofamid, (3.007) dimoxystrobin, (3.008) enoxastrobin, (3.009) famoxadon, (3.010) fenamidon, (3.011) flufenoxystrobin, (3.012) fluoxastrobin, (3.013) kresoxim-methyl, (3.014) metominostrobin, (3.015) orysastrobin, (3.016) picoxystrobin, (3.017) pyraclostrobin, (3.018) pyrametostrobin, (3.019) pyraoxystrobin, (3.020) trifloxystrobin (3.021) (2E)-2-{2-[({[(1E)-1-(3-{[(E)-1-fluoro-2-phenylvinyl]oxy}phenyl)ethylidene]amino}oxy)methyl]phenyl}-2-(methoxyimino)—N-methylacetamide, (3.022) (2E,3Z)-5-{[1-(4-chlorophenyl)-1H-pyrazol-3-yl]oxy}-2-(methoxyimino)—N,3-dimethylpent-3-enamide, (3.023) (2R)-2-{2-[(2,5-dimethylphenoxy)methyl]phenyl}-2-methoxy-N-methylacetamide, (3.024) (2S)-2-[2-[(2,5-dimethylphenoxy)methyl]phenyl]-2-methoxy-N-methylacetamide, (3.025) (3S,6S,7R,8R)-8-benzyl-3-{([3-[(isobutyryloxy)methoxy]-4-methoxypyridin-2-yl}carbonyl)amino]-6-methyl-4,9-dioxo-1,5-dioxonan-7-yl-2-methylpropanoate, (3.026) 2-{2-[(2,5-dimethylphenoxy)methyl]phenyl}-2-methoxy-N-methylacetamide, (3.027) N-(3-ethyl-3,5,5-trimethylcyclohexyl)-3-formamido-2-hydroxybenzamide, (3.028) (2E,3Z)-5-{[1-(4-chloro-2-fluorophenyl)-1H-pyrazol-3-yl]oxy}-2-(methoxyimino)-N,3-dimethylpent-3-enamide, (3.029) methyl{5-[3-(2,4-dimethylphenyl)-1H-pyrazol-1-yl]-2-methylbenzyl}carbamate.

4) Mitosis and cell division inhibitors, for example (4.001) carbendazim, (4.002) diethofencarb, (4.003) ethaboxam, (4.004) fluopicolid, (4.005) pencycuron, (4.006) thiabendazole, (4.007) thiophanate-methyl, (4.008) zoxamide, (4.009) 3-chloro-4-(2,6-difluorophenyl)-6-methyl-5-phenylpyridazine, (4.010) 3-chloro-5-(4-chlorophenyl)-4-(2,6-difluorophenyl)-6-methylpyridazine, (4.011) 3-chloro-5-(6-chloropyridin-3-yl)-6-methyl-4-(2,4,6-trifluorophenyl) pyridazine, (4.012) 4-(2-bromo-4-fluorophenyl)-N-(2,6-difluorophenyl)-1,3-dimethyl-1H-pyrazol-5-amine, (4.013) 4-(2-bromo-4-fluorophenyl)-N-(2-bromo-6-fluorophenyl)-1,3-dimethyl-1H-pyrazol-5-amine, (4.014) 4-(2-bromo-4-fluorophenyl)-N-(2-bromophenyl)-1,3-dimethyl-1H-pyrazol-5-amine, (4.015) 4-(2-bromo-4-fluorophenyl)-N-(2-chloro-6-fluorophenyl)-1,3-dimethyl-1H-pyrazol-5-amine, (4.016) 4-(2-bromo-4-fluorophenyl)-N-(2-chlorophenyl)-1,3-dimethyl-1H-pyrazol-5-amine, (4.017) 4-(2-bromo-4-fluorophenyl)-N-(2-fluorophenyl)-1,3-dimethyl-1H-pyrazol-5-amine, (4.018) 4-(2-chloro-4-fluorophenyl)-N-(2,6-difluorophenyl)-1,3-dimethyl-1H-pyrazol-5-amine, (4.019) 4-(2-chloro-4-fluorophenyl)-N-(2-chloro-6-fluorophenyl)-1,3-dimethyl-1H-pyrazol-5-amine, (4.020) 4-(2-chloro-4-fluorophenyl)-N-(2-chlorophenyl)-1,3-dimethyl-1H-pyrazol-5-amine, (4.021) 4-(2-chloro-4-fluorophenyl)-N-(2-fluorophenyl)-1,3-dimethyl-1H-pyrazol-5-amine, (4.022) 4-(4-chlorophenyl)-5-(2,6-difluorophenyl)-3,6-dimethylpyridazine, (4.023) N-(2-bromo-6-fluorophenyl)-4-(2-chloro-4-fluorophenyl)-1,3-dimethyl-1H-pyrazol-5-amine, (4.024) N-(2-bromophenyl)-4-(2-chloro-4-fluorophenyl)-1, 3-dimethyl-1H-pyrazol-5-amine, (4.025) N-(4-chloro-2,6-difluorophenyl)-4-(2-chloro-4-fluorophenyl)-1,3-dimethyl-1H-pyrazol-5-amine.

5) Compounds having capacity for multisite activity, for example (5.001) Bordeaux mixture, (5.002) captafol, (5.003) captan, (5.004) chlorothalonil, (5.005) copper hydroxide, (5.006) copper naphthenate, (5.007) copper oxide, (5.008) copper oxychloride, (5.109) copper(2+) sulfate, (5.010) dithianon, (5.011) dodin, (5.012) folpet, (5.013) mancozeb, (5.014) maneb, (5.015) metiram. (5.016) zinc metiram, (5.017) copper oxine, (5.018) propineb, (5.019) sulfur and sulfur preparations including calcium polysulfide, (5.020) thiram, (5.021) zineb, (5.022) ziram, (5.023) 6-ethyl-5, 7-dioxo-6, 7-dihydro-5H-pyrrolo[3′,4′:5,6][1,4]dithiino[2,3-c] [1,2]thiazole-3-carbonitrile.

6) Compounds capable of triggering host defense, for example (6.001) acibenzolar-S-methyl, (6.002) isotianil, (6.003) probenazole, (6.004) tiadinil.

7) Amino acid and/or protein biosynthesis inhibitors, for example (7.001) cyprodinil, (7.002) kasugamycin, (7.003) kasugamycin hydrochloride hydrate, (7.004) oxytetracycline, (7.005) pyrimethanil, (7.006) 3-(5-fluoro-3,3,4,4-tetramethyl-3,4-dihydroisoquinolin-1-yl)quinoline.

(8) ATP production inhibitors, for example (8.001) silthiofam.

9) Cell wall synthesis inhibitors, for example (9.001) benthiavalicarb, (9.002) dimethomorph, (9.003) flumorph, (9.004) iprovalicarb, (9.005) mandipropamid. (9.006) pyrimorph, (9.007) valifenalate, (9.008) (2E)-3-(4-tert-butylphenyl)-3-(2-chloropyridin-4-yl)-1-(morpholin-4-yl)prop-2-en-lone, (9.009) (2Z)-3-(4-ten-butylphenyl)-3-(2-chloropyridin-4-yl)-1-(morpholin-4-yl)prop-2-en-lone.

10) Lipid and membrane synthesis inhibitors, for example (10.001) propamocarb, (10.002) propamocarb hydrochloride, (10.003) tolclofos-methyl.

11) Melanin biosynthesis inhibitors, for example (11.001) tricyclazole, (11.002) 2,2,2-trifluoroethyl {3-methyl-1-[(4-methylbenzoyl)amino]butan-2-yl}carbamate.

12) Nucleic acid synthesis inhibitors, for example (12.001) benalaxyl, (12.002) benalaxyl-M (kiralaxyl), (12.003) metalaxyl, (12.004) metalaxyl-M (mefenoxam).

13) Signal transduction inhibitors, for example (13.001) fludioxonil, (13.002) iprodione, (13.003) procymidone, (13.004) proquinazid, (13.005) quinoxyfen, (13.006) vinclozolin.

14) Compounds that can act as uncouplers, for example (14.001) fluazinam, (14.002) meptyldinocap.

15) Further compounds, for example (15.001) abscisic acid, (15.002) benthiazole, (15.003) bethoxazin, (15.004) capsimycin, (15.005) carvone, (15.006) chinomethionat, (15.007) cufraneb, (15.008) cyflufenamid, (15.009) cymoxanil, (15.010) cyprosulfamide, (15.011) flutianil, (15.012) fosetyl-aluminium. (15.013) fosetyl-calcium, (15.014) fosetyl-sodium, (15.015) methyl isothiocyanate, (15.016) metrafenon, (15.017) mildiomycin, (15.018) natamycin, (15.019) nickel dimethyldithiocarbamate, (15.020) nitrothalisopropyl, (15.021) oxamocarb, (15.022) oxathiapiprolin, (15.023) oxyfenthiin, (15.024) pentachlorophenol and salts, (15.025) phosphonic acid and salts thereof, (15.026) propamocarb-fosetylate, (15.027) pyriofenone (chlazafenone), (15.028) tebufloquin, (15.029) tecloftalam, (15.030) tolnifanide, (15.031) 1-(4-(4-[(5R)-5-(2,6-difluorophenyl)-4,5-dihydro-1,2-oxazol-3-yl]-1,3-thiazol-2-yl) piperidin-1-yl)-2-[5-methyl-3-(trifluoromethyl)-1H-pyrazol-1-yl]ethanone, (15.032) 1-(4-(4[(5S)-5-(2,6-difluorophenyl)-4,5-dihydro-1,2-oxazol-3-yl]-1,3-thiazol-2-yl)piperidin-1-yl)-2-[5-methyl-3-(trifluoromethyl)-1H-pyrazol-1-yl]ethanone, (15.033) 2-(6-benzylpyridin-2-yl)quinazoline, (15.034) 2,6-dimethyl-1H,5H-[1,4]dithiino[2,3-c: 5,6-c′]dipyrrole-1,3,5,7(2H,6H)-tetrone, (15.035) 2-[3,5-bis(difluoromethyl)1H-pyrazol-1-yl]-1-[4-(4-[5-[2-(prop-2-yn-1-yloxy)phenyl]-4,5-dihydro-1,2-oxazol-3-yl)-1,3-thiazol-2-yl)piperidin-1-yl]ethanone, (15.036) 2-13,5-bis (difluoromethyl)-1H-pyrazol-1-yl 1-1-[4 (4-(5-[2-chloro-6-(prop-2-yn-1-yloxy)phenyl]-4,5-dihydro-1,2-oxazol-3-yl)-1,3-thiazol-2-yl)piperidin-1-yl]ethanone, (15.037) 2-[3,5-bis (difluoromethyl)-1H-pyrazol-1-yl]-1-[4-(4-{[5-[2-fluoro-6-(prop-2-yn-1-yloxy)phenyl]-4,5-dihydro-1,2-oxazol-3-yl}-1,3-thiazol-2-yl)piperidin-1-yl]ethanone, (15.038) 2-[6-(3-fluoro-4-methoxyphenyl)-5-methylpyridin-2-yl]quinazoline, (15.039) 2-((5R)-3-[2-(1-([3,5-bis (difluoromethyl)-1H-pyrazol-1-yl]acetyl)piperidin-4-yl)-1,3-thiazol-4-yl]-4, 5-dihydro-1,2-oxazol-5-yl)-3-chlorophenyl methanesulfonate, (15.040) 2-((5S) 3-[2-(1-([3,5-bis (difluoromethyl)-1H-pyrazol-1-yl]acetyl)piperidin-4-yl)-1,3-thiazol-4-yl]-4,5-dihydro-1,2-oxazol-5-yl)-3-chlorophenyl methanesulfonate, (15.041) 2-(2-[(7,8-difluoro-2-methylquinolin-3-yl)oxy]-6-fluorophenyl)propan-2-ol, (15.042) 2-(2-fluoro-6-[(8-fluoro-2-methylquinolin-3-yl)oxy]phenyl)propan-2-ol, (15.043) 2-(3-[2-(1-([3,5-bis (difluoromethyl)-1H-pyrazol-1-yl]acetyl)piperidin-4-yl)-1,3-thiazol-4-yl]-4,5-dihydro-1,2-oxazol-5-yl)-3-chlorophenylmethanesulfonate, (15.044) 2-(3-[2-(1-([3,5-bis (difluoromethyl)-1H-pyrazol-1-yl]acetyl)piperidin-4-yl)-1,3-thiazol-4-yl]-4,5-dihydro-1,2-oxazol-5-yl)phenylmethanesulfonate, (15.045) 2-phenylphenol and salts thereof, (15.046) 3-(4,4,5-trifluoro-3,3-dimethyl-3,4-dihydroisoquinolin-1-yl)quinolin, (15.047) 3-(4,4-difluoro-3,3-dimethyl-3,4-dihydroisoquinolin-1-yl)quinolin. (15.048) 4-amino-5-fluoropyrimidin-2-ol (tautomeric form: 4-amino-5-fluoropyrimidin-2(1H)-one), (15.049) 4-oxo-4-[(2-phenylethyl)amino]butyric acid, (15.050) 5-amino-1,3,4-thiadiazole-2-thiol, (15.051) 5-chloro-N′phenyl-N′-(prop-2-yn-1-yl)thiophene 2-sulfonohydrazide, (15.052) 5-fluoro-2-[(4-fluorobenzyl)oxy]pyrimidin-4-amine, (15.053) 5-fluoro-2-[(4-methylbenzyl)oxy]pyrimidin-4-amine, (15.054) 9-fluoro-2,2-dimethyl-5-(quinolin-3-yl)-2,3-dihydro-1,4-benzoxazepine, (15.055) but-3-yn-1-yl (6-[(([(Z)-(1-methyl-1H-tetrazol-5-yl)(phenyl)methylene]amino) oxy)methyl]pyridin-2-yl)carbamate, (15.056) ethyl (2Z)-3-amino-2-cyano-3-phenylacrylate, (15.057) phenazine-1-carboxylic acid, (15.058) propyl 3,4,5-trihydroxybenzoate, (15.059) quinolin-8-ol, (15.000) quinolin-8-ol sulfate (2:1), (15.061) tert-butyl (6-[(([(1-methyl-1H-tetrazol-5-yl)(phenyl)methylene]amino)oxy)methyl]pyridin-2-yl) carbamate, (15.062) 5-fluoro-4-imino-3-methyl-1-[(4-methylphenyl)sulfonyl]-3,4-dihydropyrimidin-2(1H)-one.

### Biological Pesticides a Mixing Components

The small molecules may be combined with biological pesticides.

Biological pesticides especially include bacteria, fungi, yeasts, plant extracts and products formed by microorganisms, including proteins and secondary metabolites.

Biological pesticides include bacteria such as spore-forming bacteria, root-colonizing bacteria and bacteria which act as biological insecticides, fungicides or nematicides.

Examples of such bacteria which are used or can be used as biological pesticides are:

Bacillus amyloliquefaciens, strain FZB42 (DSM 231179), or Bacillus cereus, especially B. cereus strain CNCM 1-1562 or Bacillus firmus, strain 1-1582 (Accession number CNCM 1-1582) or Bacillus pumilus, especially strain GB34 (Accession No. ATCC 700814) and strain QST2808 (Accession No. NRRL B 30087), or Bacillus subtilis, especially strain GB03 (Accession No. ATCC SD-1397), or Bacillus subtilis strain QST713 (Accession No. NRRL B-21661) or Bacillus subtilis strain OST 30002 (Accession No. NRRL B-50421), Bacillus thuringiensis, especially B. thuringiensis subspecies israelensis (serotype H-14), strain AM65-52 (Accession No. ATCC 1276), or B. thuringiensis subsp. aizawai, especially strain ABTS-1857 (SD-1372), or B. thuringiensis subsp. kurstaki strain HD-1, or B. thuringiensis subsp. tenebrionis strain NB 176 (SD-5428), Pasteuria penerrans, Pasteuria spp. (Rotylenchulus reniformis nematode)-PR3 (Accession Number ATCC SD-5834), Streptomyces microflavus strain AQ6121 (=QRD 31.013, NRRL B-50550), Streptomyces galbus strain AQ6047 (Accession Number NRRL 30232).

Examples of fungi and yeasts which are used or can be used as biological pesticides are:

Beauveria bassiana, in particular strain ATCC 74040, Coniothyrium minitans, in particular strain CON/MI 91-8 (Accession No. DSM-9660), Lecanicillium spp., in particular strain HRO LEC 12, Lecanicillium lecanii (formerly known as Verticillium lecanii), in particular strain KV0l, Metarhizium anisopliae, in particular strain F52 (DSM3884/ATCC 90448), Metschnikowia fructicola, in particular strain NRRL Y-30752, Paecilomyces fumosoroseus (new: Isarialumosorosea), in particular strain IFPC 200613, or strain Apopka 97 (Accession No. ATCC 20874), Paecilomyces lilacinus, in particular P. lilacinus strain 251 (AGAL 89/030550), Talaromyces flavus, in particular strain V 117b, Trichoderma atroviride, in particular strain SCI (Accession Number CBS 122089). Trichoderma harzianum, in particular T. harzianum nfai T39 (Accession Number CNCM 1-952).

Examples of viruses which are used or can be used as biological pesticides are:

Adoxophyes orana (summer fruit tortrix) granulosis virus (GV), Cydia pomonella (codling moth) granulosis virus ((IV), Helicoverpa anmigera (cotton bollworm) nuclear polyhedrosis virus (NPV), Spodoptera exigua (beet armyworm) mNPV, Spodoptera frugiperda (fall army worm) mNP\7, Spodoptera littoralis (African cotton leafworm) NPV.

Also included are bacteria and fungi which are added as ‘inoculant’ to plants or plant parts or plant organs and which, by virtue of their particular properties, promote plant growth and plant health. Examples include: Agrobacterium spp., Azorhizobium caulinodans, Azospirillum spp., Azotobacter spp., Bradyrhizobium spp., Burkholderia spp., in particular Burkholderia cepacia (formerly known as Pseudomonas cepacia), Gigaspora spp., order Gigaspora monosporum, Glomus spp., Laccaria spp., Lactobacillus buchneri, Paraglomus spp., Pisolithus tinctorus, Pseudomonas spp., Rhizobium spp., in particular Rhizobium trifolii, Rhizopogon spp., Scleroderma spp., Suillus spp., Streptomyces spp.

Examples of plant extracts and products formed by microorganisms, including proteins and secondary metabolites, which are used or can be used as biological pesticides are:

Allium sativum, Artemisia absinthium, azadirachtin, Biokeeper WP, Cassia nigricans, Celastrus angulatus, Chenopodium anthelminticum, chitin, ArmourZen, Uryopteris filixmas, Equisetum arvense, Fortune Aza, Fungastop, Heads Up (Chenopodium quinoa saponin extract), pyrethrum/pyrethrins, Quassia amara, Quercus, Quillaja, Regalia, rotenone, ryania/ryanodine, Symphytum officinale, Tanacetum vulgare, thymol, Triact 70, TriCon, Tropaeulum majus, Urtica dioica, Veratrin, Viscum album, Brassicaceae extract, especially oilseed rape powder or mustard powder.

### Safener as Mixing Components

The small molecules may be combined with safeners, for example benoxacor, cloquintocet (-mexyl), cyometrinil, cyprosulfamide, dichlormid, fenchlorazole (-ethyl), fenclorim, flurazole, fluxofenim, furilazole, isoxadifen (-ethyl), mefenpyr (-diethyl), naphthalic anhydride, oxabetrinil, 2-methoxy-N-({4-[(methylcarbamoyl) amino]phenyl}sulfonyl)benzamide (CAS 129531-12-0), 4-(dichloroacetyl)-1-oxa-4-azaspiro [4.5]decane (CAS 71526-07-3), 2,2,5-trimethyl-3-(dichloroacetyl)-1,3-oxazolidine (CAS 52836-31-4).

### Uses of Small Molecule Arthropod Kinin Receptor Agonists or Antagonists

### Use in Health

In the health field, i.e. the field of human medicine or veterinary medicine, the small molecules are active against parasites, in particular ectoparasites. The small molecules are preferably antagonists. The term “ectoparasite” includes typically and preferably arthropods, especially insects or acarids.

In the field of medicine, the small molecules having favorable ectotherm toxicity are suitable for controlling parasites. In the field of veterinary medicine, the parasites may occur in animal breeding and animal husbandry in livestock, breeding animals, zoo animals, laboratory animals, experimental and domestic animals. They are active against all or specific stages of development of the parasites.

Agricultural livestock include, for example, mammals, such as sheep, goats, horses, donkeys, camels, buffalo, rabbits, reindeer, fallow deer and especially cattle and pigs; or poultry such as turkeys, ducks, geese and especially chickens; or fish or crustaceans, for example in aquaculture; or, as the case may be, insects such as bees.

Domestic animals include, for example, mammals, such as hamsters, guinea pigs, rats, mice, chinchillas, ferrets, and particularly dogs, cats, caged birds, reptiles, amphibians, or aquarium fish.

In a specific embodiment, the small molecules are administered to mammals.

Use of the small molecules for the control of animal parasites is intended to reduce or prevent illness, cases of death and reductions in performance (in the case of meat, milk, wool, hides, eggs, honey and the like), such that more economical and simpler animal husbandry is enabled and better animal well-being is achievable.

In relation to the field of health, the term “control” or “controlling” in the present context means that the small molecules are effective in reducing the incidence of the particular parasite in a subject infected with such parasites to an innocuous degree. More specifically, “controlling” in the present context means that the small molecules kill the respective parasite, inhibit its growth, or inhibit its proliferation.

In the medical filed, the small molecules are administered by methods generally known in the art, such as via the enteral, parenteral, dermal, or nasal route in the form of suitable preparations. Administration may be prophylactic, metaphylactic or therapeutic.

In the veterinary field and in animal husbandry, the small molecules are administered by methods generally known in the art, such as dipping, spraying, misting or via the enteral, parenteral, dermal, or nasal route in the form of suitable preparations. Administration may be prophylactic, metaphylactic or therapeutic.

Thus, one embodiment of the present invention relates to the small molecules for use as a medicament. In other embodiments, the small molecule may be a pharmaceutically acceptable salt, hydrate, or prodrug thereof, or combinations thereof.

A further aspect relates to the small molecules for use as an antiectoparasitic agent, especially an arthropodicide, very particularly an insecticide or an acaricide.

Further aspects of the invention are veterinary medicine formulations comprising an effective amount of at least one small molecule and at least one of the following: a pharmaceutically acceptable excipient (e.g. solid or liquid diluents), a pharmaceutically acceptable auxiliary (e.g. surfactants), especially a pharmaceutically acceptable excipient used conventionally in veterinary medicine formulations and/or a pharmaceutically acceptable auxiliary conventionally used in veterinary medicine formulations.

A related aspect of the invention is a method for production of a medicine formulation as described here, which comprises the step of mixing at least one small molecule with pharmaceutically acceptable excipients and/or auxiliaries, especially with pharmaceutically acceptable excipients used conventionally in veterinary medicine formulations and/or auxiliaries used conventionally in veterinary medicine formulations.

Another specific aspect of the invention is medicine formulations selected from the group of ectoparasiticidal and endoparasiticidal formulations, insecticidal and acaricidal formulations, according to the aspects mentioned, and methods for production thereof.

Another aspect relates to a method for treatment of a parasitic infection, especially an infection caused by a parasite selected from the group of the ectoparasites mentioned here, by use of an effective amount of a small molecule in or on an animal, especially a nonhuman animal, having a need thereof.

Another aspect relates to a method for treatment of a parasitic infection, especially an infection caused by a parasite selected from the group of the ectoparasites mentioned here, by use of a veterinary medicine formulation as defined herein or on an animal, especially a nonhuman animal, having a need thereof.

Another aspect relates to the use of the small molecules in the treatment of a parasite infection, especially an infection caused by a parasite selected from the group of the ectoparasites mentioned here, in an or on animal, especially a nonhuman animal.

Another aspect relates to the use of the small molecules to control parasites by administering a sufficient amount to the parasite.

In the present context of animal health or veterinary medicine, the term “treatment” includes prophylactic, metaphylactic and therapeutic treatment.

In a particular embodiment, in this way, mixtures of at least one small molecule with other active ingredients, especially with and ectoparasiticides, are provided for the field of veterinary medicine.

The small molecules can also be used in vector control. In the context of the present invention, a vector is an arthropod, especially an insect or arachnid, capable of transmitting pathogens, for example viruses, worms, single-cell organisms and bacteria, from a reservoir (plant, animal, human, etc.) to a host. The pathogens can be transmitted either mechanically (for example trachoma by non-stinging flies) onto a host or after injection into a host (for example malaria parasites by mosquitoes).

Vector control is also possible if the small molecules are acaricide/insecticide resistance-breaking.

Small molecules are suitable for use in the prevention of diseases and/or pathogens transmitted by vectors. Thus, a further aspect of the present invention is the use of small molecules for vector control, for example in agriculture, in horticulture, in forests, in gardens and in leisure facilities, on cloths or fabrics, and also in the protection of materials and stored products.

The dosage amounts of the and administration schedule of the small molecules, or pharmaceutically acceptable salts, hydrates, or prodrugs thereof, or combinations thereof, can vary depending on other components of the composition and their effects on drug availability in a subject, the intended mode of administration, the intended schedule for administration, when other drugs are administered, any drug toxicity concerns, and the subject's response to the drug. In certain embodiments, the amount and frequency of delivery of the small molecule, or pharmaceutically acceptable salts, hydrates, or prodrugs thereof, or combinations thereof, can be such that levels in or on the subject remain well below levels at which toxicity to the recipient becomes a concern. However, the amount and frequency can also be such that the levels of the small molecule, or pharmaceutically acceptable salts, hydrates, or prodrugs thereof, or combinations thereof, in or on the subject remain continuously at a level sufficient to treat the subject for or control the parasite.

### Use on Plants or Vacant Pastures to Control Parasites

In an embodiment of the disclosure, the spread of an arthropod may be controlled or prevented by treating plant or plant parts in an area or treating a vacant pasture with a small molecule. In a preferred embodiment, the small molecule is an antagonist. For example, application directly to a plant may be used to control mites while an application to a field may be used to control the spread of ticks.

All plants and plant parts can be treated in accordance with the invention. Plants are understood here to mean all plants and populations of plants, such as desirable and undesirable wild plants or crop plants (including naturally occurring crop plants), for example forage and grasses in pastures or leguminous plants such as alfalfa, clover, etc. use to feed cattle in general, cereals (wheat, rice, triticale, barley, rye, oats), maize, soya beans, potatoes, sugar beet, sugar cane, tomatoes, bell peppers, cucumbers, melons, carrots, watermelons, onions, lettuce, spinach, leeks, beans, Brassica oleracea (e.g. cabbage) and other vegetable species, cotton, tobacco, oilseed rape, and also fruit plants (the fruits being apples, pears, citrus fruits and grapes). Crop plants may be plants which can be obtained by conventional breeding and optimization methods or by biotechnological and genetic engineering methods or combinations of these methods, including the transgenic plants and including the plant cultivars which are protectable or nonprotectable by plant breeders' rights. Plants shall be understood to mean all development stages such as seed, seedlings, young (immature) plants, up to and including mature plants. Plant parts shall be understood to mean all parts and organs of the plants above and below ground, such as shoot, leaf, flower and root, examples given being leaves, needles, stalks, stems, flowers, fruit bodies, fruits and seeds, and also roots, tubers and rhizomes. Plant parts also include harvested plants or harvested plant parts and vegetative and generative propagation material, for example cuttings, tubers, rhizomes, slips and seeds.

The inventive treatment of the plants and parts of plants with the small molecules is affected directly or by allowing the compounds to act on the surroundings, the habitat or the storage space thereof by the customary treatment methods, for example by dipping, spraying, evaporating, fogging, scattering, painting on, injecting, and, in the case of propagation material, especially in the case of seeds, also by applying one or more coats.

As already mentioned above, it is possible to treat all plants and their parts in accordance with the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (genetically modified organisms), and parts thereof are treated. The term “parts” or “parts of plants” or “plant parts” has been explained above. Particular preference is given in accordance with the invention to treating plants of the respective commercially customary plant cultivars or those that are in use. Plant cultivars are understood to mean plants having new properties (“traits”) and which have been obtained by conventional breeding, by mutagenesis or by recombinant DNA. They may be cultivars, varieties, biotypes or genotypes.

The plants and plant parts are treated with the small molecules directly or by action on their surroundings, habitat or storage space using customary treatment methods, for example by dipping, spraying, atomizing, irrigating, evaporating, dusting, fogging, broadcasting, foaming, painting, spreading-on, injecting, watering (drenching), drip irrigating and, in the case of propagation material, in particular in the case of seed, additionally by dry seed treatment, liquid seed treatment, slurry treatment, by incrusting, by coating with one or more coats, etc. It is furthermore possible to apply the small molecules by the ultra-low volume method or to inject the application form or small molecule itself into the soil.

A preferred direct treatment of the plants is foliar application, meaning that the small molecules are applied to the foliage, in which case the treatment frequency and the application rate should be adjusted according to the level of infestation with the pest in question.

In the case of systemically active ingredients, the small molecules also access the plants via the root system. The plants are then treated by the action of the small molecules on the habitat of the plant. This can be accomplished, for example, by drenching, or by mixing into the soil or the nutrient solution, meaning that the locus of the plant (e.g., soil or hydroponic systems) is impregnated with a liquid form of the small molecules, or by soil application, meaning that the small molecules according to the invention are introduced in solid form (e.g. in the form of granules) into the locus of the plants, or by drip application (often also referred to as “chemigation”), meaning that the small molecules are introduced by means of surface or underground drip tubes over particular periods of time together with varying amounts of water at defined sites in the locus of the plants. In the case of paddy rice crops, this can also be accomplished by metering the small molecules in a solid application form (for example as granules) into a flooded paddy field.

In other embodiments, the small molecule may be applied to vacant pastures to control arthropods at any stage, for example at the egg, larvae, nymph, or adult stage. The vacant lots are treated with the small molecules directly or by action on their surroundings, habitat or storage space using customary treatment methods, for example by spraying, atomizing, irrigating, evaporating, dusting, fogging, broadcasting, foaming, painting, spreading-on, injecting, watering (drenching), or drip irrigating.

Use with Baits

In an embodiment, the small molecules may be used in arthropod or animal baits. In a preferred embodiment, the small molecule is an antagonist. In a preferred embodiment, the bait is a liquid bait. The bait may be designed to attract and arthropod or another animal, such as rodents. Also included is a bait housing or trap.

Another aspect of the invention is the use of the antagonists in baits against mosquitoes. Antagonists may suppress the “satiation signal” to the brain of mosquitoes so that the small molecule antagonists could be mixed with insecticides that kill by ingestion to promote the ingestion of more of the bait. In this manner they may help the sugar bait strategies that are planned for mosquitoes or other flies (dipterans) more broadly. Antagonists when applied at micromolar concentrations may promote feeding and would be useful in “Pull strategies” promoting feeding of a bait that contains an insecticide. The antagonists when applied at very high concentration may behave as antifeedants, promoting fly-away, walk-away or jump-away behaviors and therefore, useful for “Push strategies” when in sugar baits. Agonists of the kinin receptor will likely promote aversive behaviors such as fly-away, walk-away or jump-away as previously shown for the kinin agonist 1728.

The bait composition may comprise an anti-oxidizing agent, a preservative, a coloring agent, a flavoring agent, a feed attractant, and/or an insecticide. Such additives are usually added in amounts, which are well known to the expert.

Examples of the anti-oxidizing agent are erythorbic acid, sodium erythorbate, di-tert-butyl hydroxytoluene (BHT), dl-alpha-tocophelol, nordihydroguaiaretic acid, methylhydroxyanisole, propyl gallate, guaiac resin. L-cysteine hydrochloride. Examples of the preservative are benzoic acid, sodium benzoate, salicylic acid, diphenyl, sorbic acid, potassium sorbate, dehydroacetic acid, sodium dehydroacetate, isobutyl p-oxybenzoate, isopropyl p-oxybenzoate, ethyl p-oxybenzoate, butyl p-oxybenzoate, propyl p-oxybenzoate, calcium propionate, sodium propionate, 2-methyl-4-isothiazolin-3-one (MIT), 1,2-benzisothiazolin -3-one (BIT) (mixtures of MIT and BIT are commercially available as Acticide® MBS from Thor), 1,2-Benzisothiazolin-3-one, 2-Bromo-2-nitropropane-1,3-diol or 2-Methyl-3(2H)-isothiazolone (mixtures of the latter three compounds are commercially available as acticide MBL 5515 from Thor). Examples of a coloring agent is a dye or a pigment, such as Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108, amaranth, amaranth aluminium lake, erythrosine, erythrosine aluminium lake, new coccine, Phloxine, rose bengal, acid eed, tartrazine, tartrazine aluminium lake, Sunset Yellow FCF, Sunset Yellow FCF aluminium lake, Fast Green FCF, Fast Green FCF aluminium lake, Brilliant Blue FCF, Brilliant Blue FCF aluminium lake, indigo carmine, indigo carmine aluminium lake, beta-carotene, copper chlorophyll.

Examples of the flavoring agent are cheese flavor, butter flavor, peanut flavor, peach flavor, strawberry flavor, and milk flavor.

Examples of the feed attractant are sugars, plant volatiles such as pinene and limonene, or essential oils such as olive oil, soybean oil, rapeseed oil, sesame oil, cotton seed oil, wheat germ oil, corn oil, sunflower oil, palm oil, castor oil, and linseed oil.

In a preferred embodiment, the amounts of various components of the bait composition may be selected such that a liquid bait is formed. Typically, the amounts of the components add up or may be filled up with other formulation additives to 100 wt %. Regarding the attractiveness to arthropods of the bait composition, it is known in the art that there is no difference between baits containing insecticide or without insecticide.

Any number of methods can be used to prepare the bait compositions. The method employed is dependent upon the type of formulation to be prepared, for example a gel, paste, liquid, emulsion, pressed solid, or granular.

In an embodiment, the compositions may be formulated into a liquid when added to an appropriate solvent, such as, but not limited to, water or a lipid. This may allow for long-term delivery using systems such as liquid gravity feed systems. The individual compounds may be mixed with the solvent directly, or may first be mixed, the mixture encapsulated, and then mixed with the solvent to form an emulsion or suspension of the compounds.

In some embodiments the bait may be prepared by a process comprising extruding a mixture which contains the small molecules and the bait composition. Usually, the process further comprises drying of the extruded or pelleted mixture.

Extruders are well known in the art. For example, a one screw or twin-screw extruder may be used. Also, extruders used for producing spaghetti may be used. Typically, the extrusion is accomplished at a pressure (usually taken just before entering into the extrusion grid) from 1 to 80 bars, preferably from 1 to 60 bars, and more preferably from 1 to 40 bars. Typically, the extrusion is accomplished at a temperature from 10 to 100° C., preferably from 20 to 80° C., and more preferably from 30 to 60° C. Said temperature refers to the paste during extrusion. When necessary, the temperature is maintained at the desired value by cooling. An extrusion grid may be used with holes of any shape, preferably of circular shape. Typically, the diameter of the holes is from 0.2 to 5.0 mm, preferably from 0.5 to 3 mm more preferably from 0.5 to 2.0 mm.

The extrudate may be dried to lower the water content of the extrudate. Drying may be done by the application of elevated temperatures, such as hot air, from 30 to 150° C., preferably from 50 to 80° C. The heating time depends on the temperature, the size of the extrudate and the desired amount of water in the final product.

The stick-like extrudate may be cut, e.g. with a rotating knife, into shorter sticks before or after drying, preferably before drying. In the case of circular holes, the spaghetti-shaped extrudate may be cut into cylindrical shape. In case of polygonal holes (e.g. triangular or rectangular), the extrudate may be cut into corresponding shapes. The resulting pellets might be broken into shorter granules before or after drying, preferably after drying. Preferably, the resulting granules have cylindrical shape with a length of 0.2 to 2 mm and a diameter of 0.2 to 2 mm. In another preferred embodiment, the resulting granules have a shape, which has length of 0.2 to 2 mm at its most distant points, and a diameter of 0.2 to 2 mm at its broadest diameter.

The bait may be a solid bait. Preferably, the solid bait is mixture of small solid granules. These granules may have a shape, which has length of 0.2 to 2 mm at its most distant points, and a diameter of 0.2 to 2 mm at its broadest diameter. Usually, solid state of matter is characterized by a distinct structural rigidity and virtual resistance to deformation (that is changes of shape and/or volume). Usually, solids have high values both of Young's modulus (e.g. at least 0.1 GPa) and of the shear modulus of elasticity (e.g. at least 0.01 GPa).

The compositions of the disclosure may also be encapsulated. The capsule around the compositions may be different forms, for example, in one embodiment, it can be a form of plastic with perforations or slits or easily tom, that holds the compositions encased within until pressure is released by the capsule being removed. Removal may occur, for example, by the arthropod or an animal applying pressure. In another embodiment, the capsule may be a layer that is made of a different material, natural or artificial, but will join, fuse, meld together to attractants and that will tear away from the compositions when the capsule is removed, allowing the compositions to spill out. In still another embodiment, the capsule may be a biodegradable compound, such as, but not limited to, alginate, that will deteriorate over time. The size of the capsule may be nanosized, under 1,000 nm, microsized, under 1,000 μm, or larger, under 1,000 mm, where larger capsules may form into a hydrogel matrix.

The compositions may also be placed inside a station. Any bait station may be used, for example a flat or upright station, and are known in the art. The bait station may made of plastic or a biodegradable material. The station may be a one-time use, refillable, or may be sealed after use.

The bait and/or bait housing or trap may be designed to hold the bait as well as allow either the arthropod or another animal access to the small molecules. By way of nonlimiting example, the bait housing or trap may comprise the small molecules and a bait, where the bait is designed to attract a rodent or other animal and to apply the small molecule to the body of the animal. The animal may then spread the small molecules over an area.

### Assays

The small molecules, either agonists or antagonists, disclosed herein may also be used in assays relating to kinin receptors, preferably arthropod kinin receptors. However, non-arthropod receptors may also be used to check for binding similarities between arthropod and non-arthropod kinin receptors.

In a preferred embodiment, the assay is a cell-based assay expressing an arthropod kinin receptor, which is a G protein-coupled receptor (GPCR). Upon binding of an agonist, GPCRs coupled with different Gα proteins that trigger different downstream cellular signaling pathways and result in changes in concentration of second messengers, such as calcium ions (Ca²⁺) (receptors coupled to Gα_{q/11}, such as the kinin receptor) or cyclic adenosine monophosphate (cAMP) (Gαⱼ/Gαₛ), or regulate the activity of Rho GTPase nucleotide exchange factors (Gα_{12/13}).

In ticks, for example, the kinin receptor couples to Gα_{q/11} protein and, therefore, a fluorescence intracellular calcium mobilization assay may be used.

In other embodiments, the small molecules may be labeled. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with carbon-13 (¹³C), or nitrogen-15 (¹⁵N). In another example, the small molecules may be labeled with a fluorescent dye, and amines or other chemical tags. Labeled small molecules may be used for measuring expression or detecting the location of the receptors.

In other embodiments, the small molecules may be used in tissue or organ assays to detect physiological changes relating to either agonists or antagonists. As shown in the Examples, the small molecule antagonists of the kinin receptor decrease the contraction frequency of the mosquito Aedes aegypti hindgut, consistent with the role of kinins being myotropic in insects. Therefore, the molecules can be used in assays with arthropod muscles and to study arthropod muscle physiology.

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

### EXAMPLES

### Example 1

### 1. Introduction

The southern cattle tick. Rhipicephalus microplus, is one of the most important livestock pests.¹ It is the primary vector of the deadly disease agents Babesia spp. And Anaplasma spp.² Disease prevention relies heavily on tick control and as a consequence of the increasing acaricide resistance detected worldwide,³ there is an urgent need for novel targets for acaricide development. Despite being eradicated from the United States since 1943, this tick species is well-established in other tropical and subtropical countries and causes enormous economic losses to the local cattle industry. Cattle fever ticks (R. annulatus and R. microplus) have always been a threat to the US cattle industry owing to proximity to endemic populations in Mexico. Despite quarantine efforts, R. microplus ticks are being found at a considerable distance north of the tick eradication quarantine area and intercepting these ticks is crucial, especially because a high proportion of these ticks are resistant to permethrin.⁷ The G protein-coupled receptors (GPCRs) are the largest family of transmembrane protein in metazoans. They transduce diverse extracellular signals including light, protons, hormones, neuropeptides, glutamate and lipoglycoproteins.⁸ GPCRs are viable targets as >30% of human drugs bind to this receptor superfamily.⁹ The success of amitraz as an acaricide acting on the arthropod octopamine receptor provides the proof-of-principle for GPCRs as suitable targets for tick control.¹⁰ Likewise, GPCRs for small neuropeptides could be utilized for pest control by finding small molecules with agonist or antagonist activity).^{11,12}

Insect kinins are candidate small molecules that initially were identified for their myotropic activity in the cockroach hindgut,¹³ and then proved to be pleiotropic. Kinins regulate diuresis,¹⁴ gut enzyme release,¹⁵ sugar taste perception in contact chemosensory neurons in Aedes aegypti,¹⁶ and tracheal clearance and air filling before ecdysis in Drosophila.¹⁷ The exact physiological functions of the kinin system in ticks remain unknown. In a recent study, silencing of the tick kinin receptor in R. microplus females significantly decreased reproductive fitness, lowered the percentage of egg hatching, and—interestingly—discolored the midguts, which presumably is linked to interference with heme uptake.¹⁸ Our previous study predicted 17 tick kinins from the R. microplus kinin precursor peptide¹⁹; 14 of the shorter kinins were synthesized and tested on the recombinant tick kinin receptor, and all were agonists.²⁰ However, peptides are expensive to mass-produce and susceptible to enzymatic degradation.²¹ Therefore, the overall goal of this study was to identify small molecules as surrogates of peptide agonists and antagonists (blockers) of the tick kinin receptor to interfere with the function of the kinin signaling system. Antagonists of the tick kinin receptor are hypothesized as useful tools to mimic the detrimental reproductive effects obtained by its silencing.¹⁸

Upon binding of an agonist, GPCRs coupled with different Gα proteins that trigger different downstream cellular signaling pathways and result in changes in concentration of second messengers, such as calcium ions (Ca²⁺) (receptors coupled to Gα_{q/11}, such as the kinin receptor) or cyclic adenosine monophosphate (cAMP) (Gαᵢ/Gαₛ), or regulate the activity of Rho GTPase nucleotide exchange factors (Gα_{12/13}).⁸ To identify ligands of a GPCR, monitoring the titer of the second messenger upon activation is among the most widely used methodologies.²² The tick kinin receptor couples to Gα_{q/11} protein²³ and, therefore, a fluorescence intracellular calcium mobilization assay was developed with the tick kinin receptor stably expressed in a CHO-K1 cell line.²⁰ Here the fluorescence assay in 384-well plate format was utilized to screen a small molecule library in high-throughput mode, and consisted of a ‘dual-addition’ for antagonist identification.¹⁹ High-throughput screening (HTS) of a target GPCR is a common method with which to discover new drugs.²² Chemical library selection for HTS on a tick neuropeptide GPCR is problematic owing to the great evolutionary differences between invertebrates and humans. Screening of drugs that are active on the most similar human receptors often yields low activity in the arthropod receptor counterpart, as we found previously by screening an antagonist library of the human neurokinin receptors on the tick kinin receptor, as they are not orthologs.¹⁹

Here, we utilized a random screening approach to discover novel ligands of the kinin receptor in 1-ITS mode, yielding excellent Z′-values, and applying a stringent threshold for hit selection. The screening of a random small molecule library yielded the identification of novel tick kinin receptor antagonists. We complemented this approach with successful structurally based in silico searches of larger libraries of small molecules. A hindgut assay further confirmed the antagonistic effect of three small molecules, SACC-0412060, SACC-04122062 and SACC-0412066, as they inhibited the increased rate of muscle contraction elicited by the potent kinin agonist analog 1728.

### 2. Materials and Methods

### 2.1 Preparation of the Small Molecule Library in ‘Drug Plates’

Small molecules utilized for this study were part of a Texas AgriLife Research compound library in the laboratory of James Sacchettini, designated the SAC-2 library. This library is composed of randomly selected small molecules purchased from commercial vendors with chemical structures chosen for breadth of structural diversity and drug-likeness. Because the library is large (>100,000 compounds), it provides the opportunity to physically screen a portion of the library and subsequently screen the entire library in silico using structural similarity to the experimentally found hit molecules. The molecules in stock plates were prepared in 100% dimethyl sulfoxide (DMSO) at ˜1 mmol L⁻¹ in 384-well plates and stored at −20° C. Compound plates for screens (hereafter drug plates) were prepared from the stock plates at 1:10 dilution in 384-well plates (Corning® 3680, Corning, NY, USA) by transferring 3 μL stock plate solution into each well containing 27 μL Dulbecco's phosphate-buffered saline (DPBS) (Corning® 21-031-CV) for a 100 μmol L⁻¹ final compound concentration in 10% DMSO in DPBS. These plates were stored at 4° C. before use and are referred to as ‘drug plates’.

### 2.2 Cell Culture

The target receptor, R. microplus kinin receptor, is stably expressed in the recombinant CHO-K1 cell line referred to as BMLK₃, and was constructed and selected as described previously.²³ Cells transfected only with empty plasmid as control are referred to as ‘vector-only’ (VSO). BMLK₃ or V/O cells were cultured in T-75 flasks with selective medium (F-12 K medium containing 10% FBS and 800 μg mL⁻¹ of G418 sulfate) for one to two passages before the experiment. All cells were incubated overnight at 37° C. and 5% CO₂ in a humidified incubator.

### 2.3 Kinin Receptor Functional Calcium Fluorescence Assay

An intracellular calcium fluorescence assay was conducted as described previously.²⁰ Briefly, when the cells reached ˜90% confluency,²⁴ they were trypsinized and suspended in F-12 K medium containing 1% FBS and 400 μg mL⁻¹ G418 sulfate at 4×10⁵ cells mL⁻¹ to be seeded in 384-well plates with black walls and clear bottom (Greiner®, 781 091, Greiner Bio-One, Kremsmunster, Austria), coated with Poly-D-lysine (Sigma-Aldrich, St Louis, MO, USA). The cell suspension (25 μL; ˜10,000 cells well⁻¹) was dispensed into each of the 384 wells of the plate. Unless specified, the pipet ling steps for the 384-well plate fluorescence assay were performed by an Integra Viaflo® system equipped with 384-pipetting head (384/12.5 μL) (Integra Bioscience, Hudson, NH, USA) or a CyBlo® Well Vario System (384/60) (Analytik Jena AG, Jena, Germany); both allowed simultaneous addition of liquid into the 384-well plate. The plates were incubated overnight at 37° C. and 5% CO₂ in an incubator. On the second day, the loading dye (1×) was prepared by diluting FLUOFORTE®(Enzo Life Sciences, E Farmingdale, NY, USA) dye into the assay buffer (1:1000), according to the manufacturer's instructions. The assay buffer consists of 1×HHBS (Hank's buffer with 20 mmol L⁻¹ HEPES) and dye efflux inhibitor (9:1). The old media in the 384-well plate was disposed of by inverting the plate on tissue paper, and replaced by 25 μL of loading dye (1×). The plate then was incubated at 37° C. for 30 min and equilibrated at room temperature (RT) for another 30 min before the cells were ready for HTS.

### 2.4 High-Throughput Screening

The ‘dual-addition’ assay that we developed here allows identification of agonist or antagonist in the same assay in a high-throughput mode.¹⁹ The theory of the ‘dual-addition’ assay is reviewed elsewhere.²⁵ In brief, the first addition of the test compound into the cell plate allowed identification of potential agonists when a higher fluorescence signal was detected compared to the solvent control. The second addition of agonist kinin peptide was applied after the cells were incubated with the test molecule for 5 min. This allowed the identification of potential antagonists when a lower fluorescence signal was detected compared to the control (solvent+agonist kinin peptide).

Before beginning the HTS, each plate containing cells and 25 μL assay buffer per well was read to obtain an endpoint fluorescence value (relative fluorescence units, RFU) as background signal. For HTS, 0.5 μL of the compounds in drug plates were dispensed into cell assay plates to reach a final concentration of 2 μmol L⁻¹ in 0.2% DMSO. Secondly, the addition of 500 nmol L⁻¹ of agonist FFFSWGa (JPT Peptide Technology, Acton, MA, USA) was performed after a 5 min incubation with the compound. The fluorescent signal was read at an excitation/emission wavelength of 495/525 nm in plate end-point mode with a Clariostar® plate reader (BMG Labtech, Ortenberg, Germany). The cell responses were read immediately after the first addition, and 5 min after the second addition. For both readings, the plate was read from both forward and reverse orientations to compensate for the decrease in signal strength in the kinetic assay during plate reading, because there was a 1 min lag time between the readings of the first and the last well. The fluorescent cellular responses to both compound addition and agonist addition were represented as the average of two values that were obtained by the forward and reverse plate readings, subtracting the background signal. Therefore, only one value represents each compound. In this study, 63 drug plates containing 19,760 unique small molecules were screened on the BMLK₃ cell line.

### 2.5 Quality Control for HTS

The quality of each HTS assay in the 384-well plate was evaluated through the calculation of the Z′-factor, which involves the responses in RFU of only both negative and positive controls as indicators of the assay condition independently of the test compounds.²⁶ This value reflects both the error associated with the controls of a screen, and the size of the ‘hit window’. A Z′-factor ≥0.5 indicates a quality screen with a well-defined hit window.²⁷ Z′-factors used in this study are reported in supporting information, Table 1. Assays with Z′-factor <0.5 were discarded.

$${Z}^{′} = 1 - \frac{( 3 {σ}_{c +} + 3 {σ}_{c -} )}{\vert {μ}_{c +} - {μ}_{c -} \vert} \qquad Equation ( I )$$

Here μ_{c-} and μ_{c+} represent the average RFUs of the negative control (blank solvent, n=64; i.e. 4 columns×16 wells) and the positive control (blank solvent+agonist; these are the same 64 wells used as negative controls, but read again after the second injection) wells, respectively. And σ_{c-} and σ_{c+} represent their corresponding standard deviations (SDs). Therefore, 320 wells remained available for compound testing.

### 2.6 Hit Molecule Selection

The cut-off for selecting agonist hits was set to a normalized percent activation (NPA) max ratio >46%. The NPA is calculated as the percentage of the normalized response of the particular agonist relative to the normalized response of the control kinin receptor agonist (FFFSWGa at 500 nmol L⁻¹), which was regarded as the maximal response (NPA=100%) from the BMLK₃ cell line.²⁸ The raw fluorescence signal of each well (RFU_{ago} or RFU_{c+}) was subtracted by the respective background signal (RFU_{bg}), as follows:

$$NPA = \frac{{RFU}_{ago} - {RFU}_{bg}}{{RFU}_{c +} - {RFU}_{bg}} \qquad Equation ( II )$$

The cut-off for selecting antagonist compounds was set to an observed inhibitory activity (Iₒ)>42%. The inhibitory activity was estimated as the percentage of the normalized response to the agonist peptide of the cells preincubated with the antagonist (RFUₐₙₜ-RFU_{bg}), relative to the normalized response to the agonist peptide of the cells preincubated with blank solvent (positive control).

$${I}_{o} = 1 - \frac{{RFU}_{ant} - {RFU}_{bg}}{{RFU}_{c +} - {RFU}_{bg}} \qquad Equation ( III )$$

### 2.7 Hit Validation in Dose-Response Assay

Agonist and antagonist hit molecules obtained from the HTS with the criteria described above subsequently were validated in a dose-response assay on the BMLK₃ cells and V/O cells using the same calcium fluorescence assay as described above (Section 2.3).

The analysis of the dose-response of each compound was performed using 20 dosages, starting from 25 μmol L⁻¹ (as final concentration in the assay plate) to 28 nmol L⁻¹, resulting from a serial dilution factor of 1:1.4. Hit molecules were obtained from 96-well master plates where the concentration of each molecule is ˜10 mmol L⁻¹ in 100% DMSO. Two microliters of these solutions were added into wells of the third column in the 384-well-plate, containing 78 μL DPBS, 6.67% DMSO, as the starting concentration (250 μcool L⁻¹) for serial dilutions. The rest of the 19 serial dilutions were in 10% DMSO in DPBS in these drug plates, and the final DMSO concentration in the assay plate was 1%.

The dose-response assays were performed with the same dual-addition assay described above, except that 1 μcool L⁻¹ of Rhimi-K-1 (QFSPWGamide, Genescript® Biotech, Piscataway. NJ, USA), an R. microplus endogenous kinin, was added in the second addition to achieve unbiased validation of antagonistic activity. The EC₅₀ and IC₅₀ were determined by a nonlinear regression fit, using the Hill equation for a concentration-response series of data in Collaborative Drug Discovery (CDD) vault (www.collaborativedrug.com).²⁹ Screening of the same compounds on the V/O cells allowed identification of false agonist hits, which were discarded. False hits were those that induced fluorescence responses in V/O cells, through likely activation of endogenous CHO-K1 cell receptors.

### 2.8 Virtual Hits and Analogs

Utilizing a chemical search tool available in the CDD vault, each of the molecules validated by dose-response assays were used as query for in silico searches of the Texas AgriLife library (389,957 molecules) to identify virtual hits with ≥70% Tanimoto structural similarity. This was performed using the ChemAxon's MarvinJS directly in the ‘explore data|search page’ in CDD. Additionally, seven analogs of the most potent antagonist hit (SACC-0064443) were purchased from ChemBridge™ for studying the structure-activity relationships. These analogs were validated in the 20-dosage serial dilution assay, as described above.

### 2.9 Cytotoxicity Assay

Human dermal fibroblasts (HDF) cells were used to evaluate the potential cytotoxicity of the hit molecules, following the assay protocol as described previously.³⁰ In brief, the cells were cultured in DMEM (Lonza) media supplemented with 10% fetal bovine serum (Lonza) and penicillin/streptomycin (10 U mL⁻¹) (Lonza). On the day of assay, HDF cells were trypsinized, counted and resuspended at a density of 64,000 cells mL⁻¹ in the media, and test compound stocks were prepared at 800 μmol L⁻¹ in 10% DMSO in PBS. Cells (39 μL) were plated in a 384-well plate (Corning, 3680), 1 μL of compound was added into each well (three pseudo-replicate wells for each assay) at a final concentration of 20 μmol L⁻¹ in 0.25% DMSO and incubated at 37° C. After 48 h, 4 μL of resazurin dye (0.2 mg mL⁻¹) were added and the assay plates were incubated for an additional 24 h. The next day the fluorescence of the resazurin was measured on a microplate reader (BMG Labtech) to assess cell death by obtaining the percentage of fluorescence signal decreased by comparing cells incubated with compound to the cells incubated with buffer control. The fluorescence intensity is proportional to the number of live cells. The HDF cytotoxicity was expressed as the average percentage of cell growth inhibition in the compound treated wells compared to the control wells. The cut-off threshold for cytotoxicity was 10%.

### 2.10 Validation of Antagonistic Activity on the Recombinant Mosquito Kinin Receptor

Three identified potent small molecule antagonists, and as control, a small molecule of very low antagonistic activity, all identified as part of this study, were tested in the calcium fluorescence assay, as above, for their activity on the cell line expressing the Aedes aegypti kinin receptor (IGKN G12 cell line). Receptor cloning, expression and clonal cell selection were as described previously.³′ The recombinant tick kinin receptor (BMLK₃) was tested simultaneously as positive control for the assay. BMLK₃ and IGKN G12 cells were seeded in 96-well-plates (Greiner Bio-One®, B/C 655097) at a density of 4×10° cells in 100 μL maintenance medium. Cells were prepared as described above for the calcium fluorescence assay; and each well was added with 90 μL assay buffer (see Section 2.3, HHBS plus dye efflux inhibitor).

For each compound three 10× concentrations (100, 300, 1000 μmol L⁻¹) were prepared and 10 μL of each was added manually into one well of each cell line for a final concentration of 10, 30 and 100 μcool L⁻¹. After 5 min incubation at room temperature, a kinin receptor agonist peptide, the kinin agonist analog 1728²⁸ [[Aib]FF[Aib]WGamide, RoyoBiotech, Shanghai, China] was added manually (50 μL; 3× stock in HHBS) into each well for a final concentration of 1 μmol L⁻¹. The cellular calcium response in RFU was immediately recorded continuously for 60 s with 3 s intervals between reads using a Clariostar plate reader. The average RFU of these 20 reads represented the raw response to each treatment. Three independent replicates were run for each concentration and compound. The percentage of signal reduction with respect to the control that contained solvent and the kinin agonist analog 1728 was calculated using the average RFU registered for the treatment with each small molecule divided by the average RFU recorded for the control (100%). Data analyses were performed with the percentage reduction of the three independent replicates. PRISM (Graphpad, San Diego. CA, USA) was utilized for analysis and graphics.

### 2.11 Validation of Antagonistic Activity in the Hindgut Contraction Inhibition Assay

A mosquito colony of Ae. aegypti (Diptera: Culicidae), Liverpool strain, was maintained at 26.5° C. in a 16 h:8 h, light:dark photoperiod and provided with 10% sugar water, as described previously.³¹ For the experiment, 3-5-day-old females were used, and the assay was modified based on our previous protocol.³³ A group of six female mosquitoes were cold-anesthetized on a Petri dish placed on ice, where both wings were removed. Mosquitoes became active when the plate was removed from the ice. The dissection dish was filled with Ringer's solution (150 mmol L⁻¹ NaCl, 25 mmol L⁻¹ HEPES, 3.4 mmol L⁻¹ KCl, 7.5 mmol L⁻¹ NaOH, 1.8 mmol L⁻¹ NaHCO₃. 1 mmol L⁻¹ MgSO₄, 1.7 mmol L⁻¹ CaCl₂ and 5 mmol L⁻¹ glucose, pH 7.1) at room temperature. To dissect the hindgut of each mosquito, their head and thorax were removed with forceps, and the abdomen was submerged in the Ringer solution. The hindgut, ovaries and Malpighian tubules (MTs), and midgut were taken out by pulling the last segment of the abdomen under the Ringer solution. Then, the ovaries and the majority of the midgut were removed using scissors leaving the intact hindgut and MTs. The isolated tissue was transferred with forceps, gripping on the cuticle of the last segment, into a drop of 30 μL Ringer solution in a 12-well-plate (Greiner Bio-One®, 665,102). After six mosquitoes were dissected as a group, paraffin oil (1-2 mL) was added to each well to cover the saline. The cuticle of the last abdominal segment was drawn out of the Ringer solution into the oil for better observation of the hindgut contraction. The tissues were filmed using an Olympus SZ60 camera (Olympus America Inc., Center Valley, PA, USA) mounted on a dissecting microscope.

First, the hindgut basal contraction rate was recorded by filming each tissue for 60 s. Secondly, 15 μL of 3× small molecule (300 μmol L⁻¹) solution or corresponding solvent-buffer (3% or 10% DMSO in Ringer solution) was added to the drop of Ringer solution containing each tissue for a final concentration of 100 μmol L⁻¹. After 5 min of incubation at room temperature, 15 μL kinin agonist analog 1728 solution (4×in Ringer solution) was added to the Ringer solution to reach a final concentration of 10 μmol L⁻¹. After 1 h of incubation, the tissue was filmed again for 60 s.

The contractible hindgut of mosquito consists of a funnel-shaped pylorus located immediately after the posterior midgut, followed by a long, coiled ileum, and a bulbous colon which contains the rectal pads, and a tapering rectal tube at the posterior end.³⁵ To analyze each video, the hindgut contraction number was counted by observing the contraction occurring at the junction between the pyloric sphincter and the most anterior ileum. The hindgut contraction rate was expressed as the number of contractions observed over 60 s. The fold change in contraction rate was calculated as the contraction rate estimated at 1 h post treatment divided by the basal rate estimated before the treatment with the chemicals. Each treatment was repeated with 30 female mosquitoes independently. All statistical analyses were done by PRISM software (v8, GraphPad). The differences in fold-change of the contraction rates among treatments were analyzed using the nonparametric Kruskal-Wallis test, followed by the Dunn's multiple comparison test. For only one small molecule. SACC-0412062, that had to be dissolved using a higher DMSO concentration (10%), the difference in fold-change of the contraction rate between the control Ringer-solvent treatment and each compound treatment was analyzed by the Mann-Whitney U-test, a nonparametric test.

### 2.12 Blast Analyses of Receptors

Blast searches of the ‘Genomic+transcript databases’ of Acari were conducted in NCBI using the R. microplus kinin receptor mRNA sequence (AF228521.1) as query. The tick kinin receptor deduced amino acid sequence (ADM47603.1) was used as query in NCBI against the Acari protein database to identify similar tick receptors.

### 3. Results

### 3.1 Identification of 58 Hit Molecules Through HTS

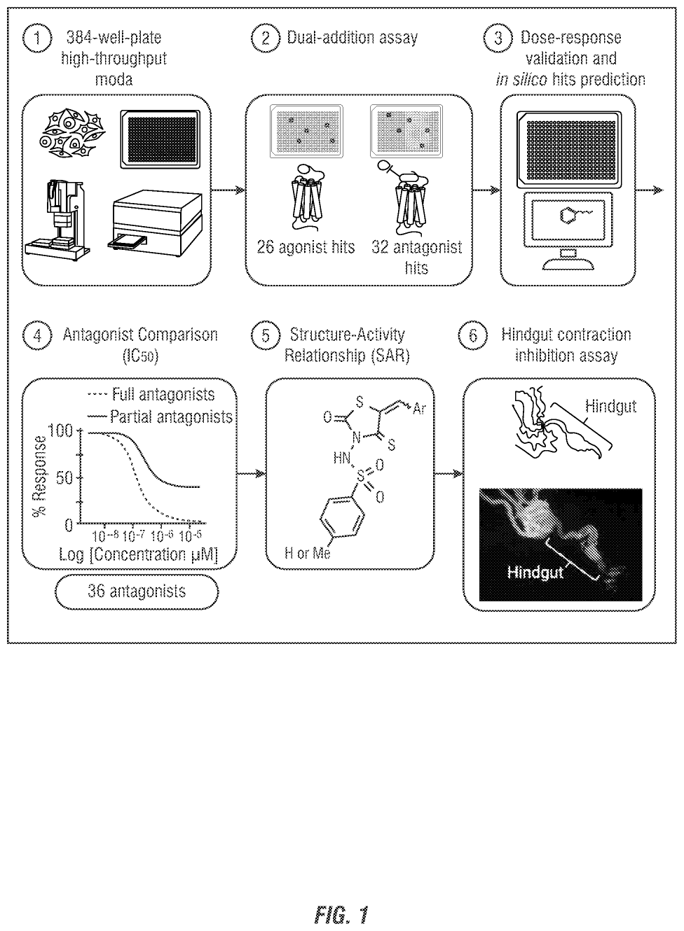

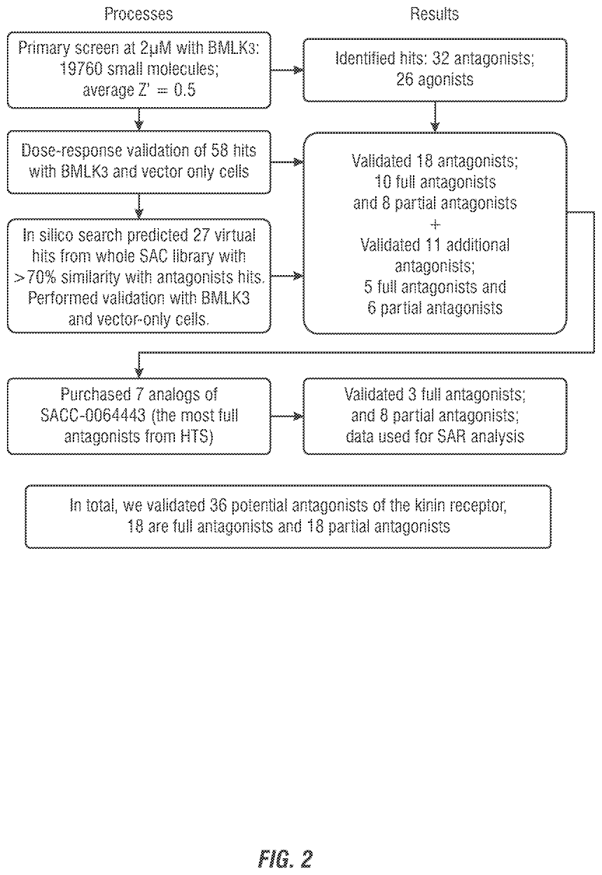

The pipeline for this novel ligand identification through HTS is outlined in FIG. 1. We developed a dual-addition calcium fluorescence assay in 384-well mode on the CHO-K1 cells permanently expressing kinin receptor from the southern cattle tick R. microplus. In the process, relevant variables were validated (data not shown), such as cell density (10,000 cells well⁻¹), concentration of the screened compounds (2 μmol L⁻¹), final DMSO concentration (0.2%) in the assay plate, agonist peptide (a generic kinin analog, FFFSWGa) concentration (500 nmol L⁻¹), pipetting speed and depth (CyBio® and Viaflo programs in Appendix Si) and reading time post-addition of the agonist peptide (5 min). The results of HTS and dose-validation are summarized in FIG. 2. A total of 19,760 small molecules were screened with this assay and the hit ratio was 0.29% with 58 hit molecules selected (Table 2) according to the criteria described in Section 2. Among them, 26 small molecules were identified as potential agonists (NPA≥46%) and 32 small molecules as potential antagonists (Iₒ≥42%).

### 3.2 Validation of Antagonists Through Dose-Response Assay

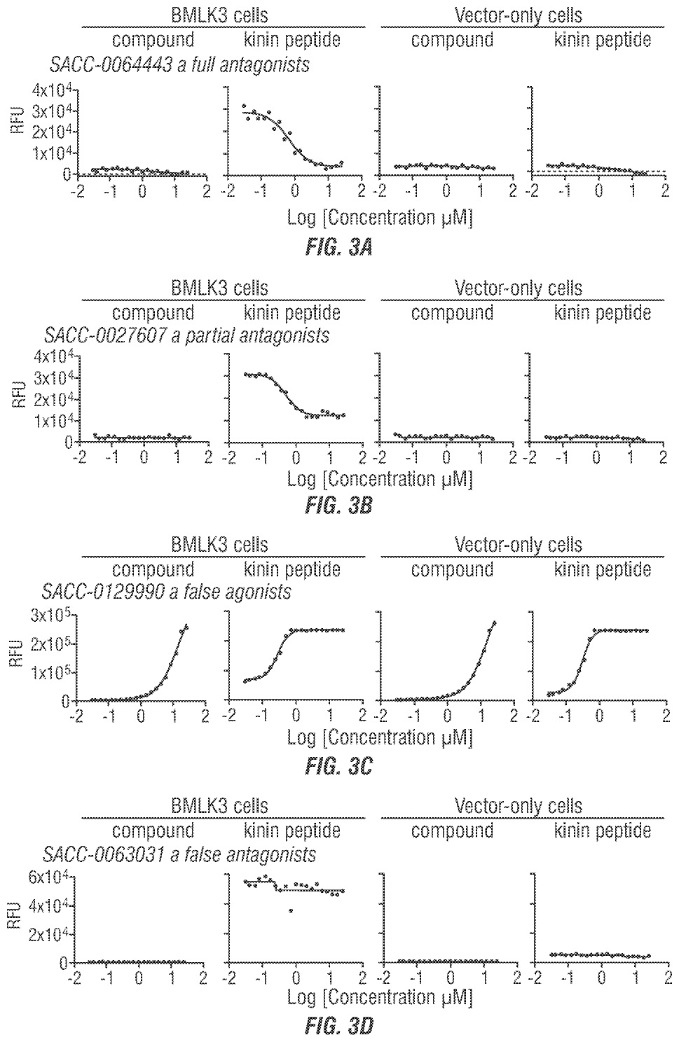

These 58 molecules were validated for their dose-dependent response on both BMLK₃ cells and V/O cells (lacking the tick kinin receptor). A summary of hit molecule characterization through dose-response curves is shown in FIG. 3. The screening of compounds on the V/O cells assisted with the exclusion of the off-target agonists when the V/O cells respond to a compound in a similar way to the BMLK₃ cells [e.g. FIG. 3C]. Our results suggested that all of the 26 putative agonists which we identified were either off-target or not active (curves in Table 3). The dose-response assay confirmed 18 off-target agonists as the V/O cells produced similar dose-responses as with the BMLK₃ cells. The remaining 22 hit molecules (putative agonists and antagonists) did not show dose-response activities [FIG. 3D]. By contrast, 18 molecules showed dose-dependent antagonistic activities. Ten molecules acted as full antagonists that fully inhibited the cellular response to the tick kinin, Rhimi-K-1 [e.g. FIG. 3A] and eight molecules acted as partial antagonists, as the response to kinin peptide was not completely suppressed even at high concentration (Table 3 and FIG. 3B as an example).

### 3.3 Antagonists Identification Through an in Silico Screen

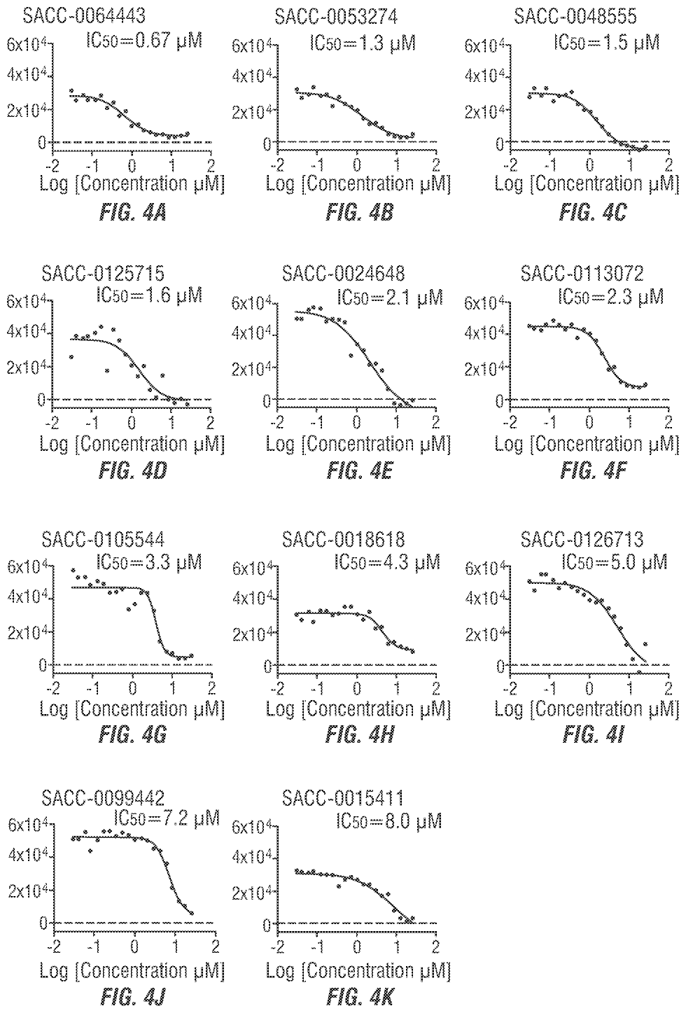

A structure-based in silico screen on the remaining existing chemical library, comprising 389,957 small molecules, identified 27 small molecules with ≥70% structural similarity to the experimentally identified antagonists (FIG. 2 and Table 4). These molecules were subsequently validated in the same dose-response assay on the BMLK₃ and V/O cells. As a result, 16 additional antagonists (five full antagonists and six partial antagonists) were identified (FIG. 2 and Table 4). In summary, 15 full antagonists and 14 partial antagonists were validated from hits identified in HTS and in silico prediction. Among the 15 full antagonists, nine small molecules had IC₅₀<5 μmol L⁻¹ [FIGS. 4A-I], two had IC₅₀ between 7 and 8 μmol L⁻¹ [FIGS. 41 and 4K], and three were less potent, with IC₅₀>10 μmot L⁻¹ (Table 6).

### 3.4 Structure-Activity Relationship Analysis

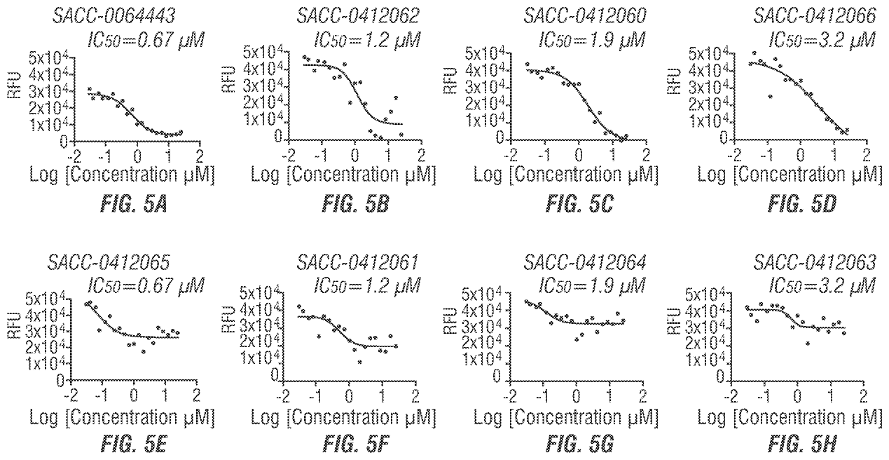

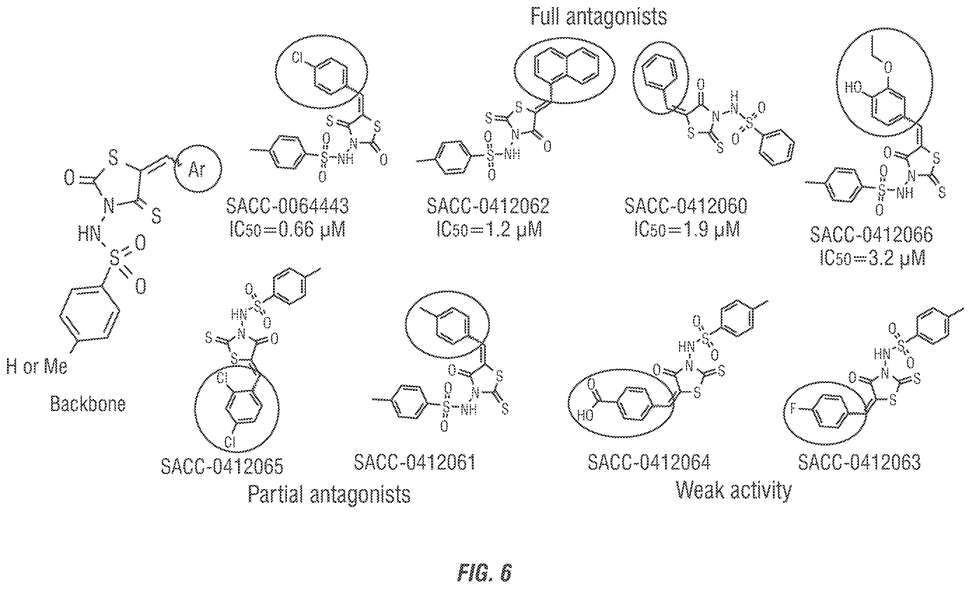

One of the most potent antagonists identified from HTS, SACC-0064443 [IC₅₀=0.67 μmol L⁻¹; FIG. 4A], was selected to study structure-activity relationships (SAR). One small molecule (SACC-0412066) with 95% structural similarity to SACC-0064443, and six 2D and 3D analogs of SACC-0412066 were obtained from ChemBridge™ Chemical. They were tested in the dose-response assay in the same fashion as mentioned above (Table 5). Three compounds showed full antagonistic activity with IC₅₀s ranging from 1.2 to 3.2 μmol L⁻¹ [FIGS. 5B-D], which were equivalent to the activity to SACC-0064443 [FIG. 5A]. Two compounds were partial antagonists [FIGS. 5E and 5F] and the other two showed very weak antagonistic activity [FIGS. 5G and 5H]. Based on this series of results, we predicted a backbone of tick kinin receptor antagonist with variable side chain (R-group) (FIG. 6, structure on the far left). The results suggested that the R-group is selected from Phenyl, naphthyl, 4-Me-phenyl, 4-Cl-phenyl, 4-F-phenyl, 4-carboxy-phenyl or 4-OH-groups. Among them, phenyl and naphthyl were best for activity, whereas substitution on phenyl decreased activity (FIGS. 5 and 6). In addition, to determine the mammalian cellular toxicity of the 36 antagonists that we validated from dose-response assay, a cell toxicity assay was performed with human epidermal fibroblast cells. Thirty-two of 36 compounds showed no cytotoxicity with human dermal fibroblast (HDF) fluorescence signal inhibition <10% when tested at 20 μcool L⁻¹ (Table 6). Two of the potent antagonists, SACC-0053274 and SACC-0015411 [curves in FIGS. 4B and 4K], showed low toxicity with 10-11% HDF inhibition. The remaining SACC-0050177 and SACC-0029037 had a HDF inhibition >10% and they exhibited low activity on the receptor, and therefore were not pursued.

### 3.5 Antagonistic Activity Validation on the Recombinant Mosquito Kinin Receptor

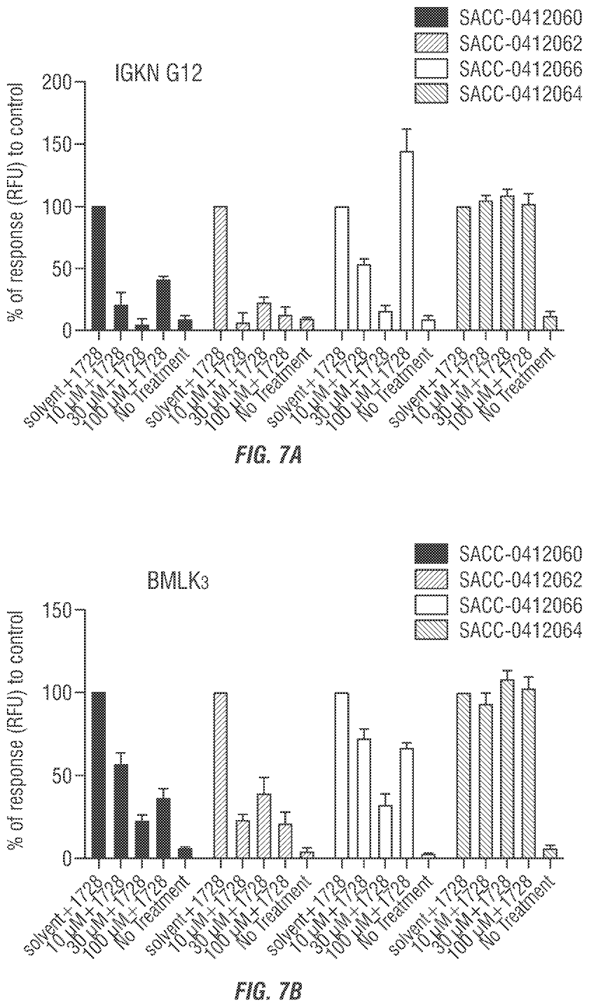

The most potent commercially available antagonists, SACC-0412060, SACC-0412062, and SACC-0412066 [FIGS. 5B-5D] were selected to be tested and SACC-0412064 was used as a negative control for its weak activity on the tick recombinant receptor [FIG. 5G]. Before the tissue bioassay, we first validated the in vitro antagonistic activity of these four molecules on the recombinant mosquito kinin receptor (IGKN G12). Through results of the calcium mobilization fluorescence assay, three active molecules showed antagonistic activities on IGKN G12 similar as on the BMLK₃ cells. Inhibition was calculated as the percentage inhibition of the maximal response that was obtained by applying solvent in the first addition and the kinin agonist analog 1728 in the second addition of the assay (FIG. 7, first bar for each compound). All three tested concentrations (10, 30 and 100 μmol L⁻¹) of SACC-0412060 and SACC-0412062 inhibited ≥50% of this maximal response [FIG. 7A]. SACC-0412066 at 30 μmol L⁻¹ inhibited 84% response on IGKN G12 compared to the control [FIG. 7A]. Unexpectedly, this molecule elicited a strong calcium response at 100 μmol L⁻¹, indicative of receptor activation. It is possible that at this high supraphysiological concentration, other endogenous receptors could have been activated. We did not explore this response further. The control molecule (SACC-0412064) did not antagonize the calcium response even at 100 μcool L⁻¹ (FIG. 6, bars in violet).

### 3.6 Antagonistic Activity Validation in the Mosquito Hindgut Contraction Inhibition Assay

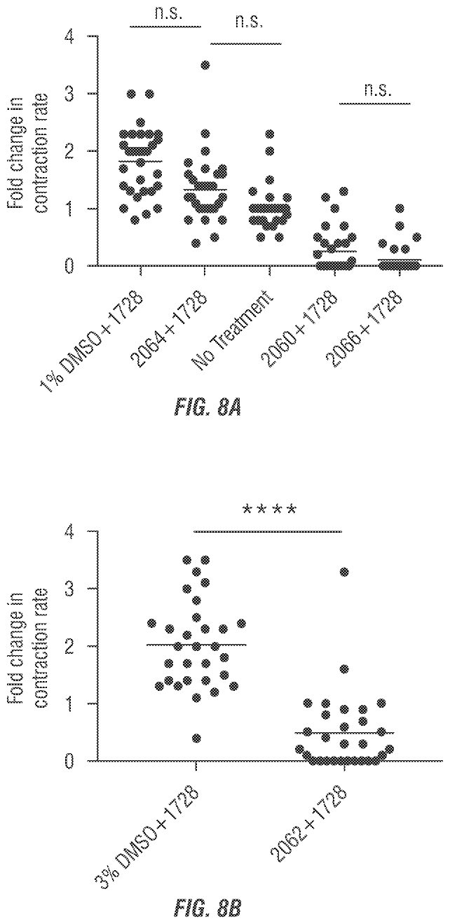

In order to validate the bioactivity of these small molecule antagonists, the compounds were tested for inhibition of the canonical myotropic activity of the kinin peptide using a hindgut contraction inhibition assay. For the hindgut contraction inhibition assay we first confirmed the contractile activity of the kinin agonist analog 1728 on mosquito hindgut. In this assay we detected significant differences among treatments (Kruskal-Wallis test) and the Dunn's test revealed a significant (-two-fold) increase in contraction rate in hindguts incubated with 10 μmol L⁻¹ 1728 in 1% DMSO 1 h post-addition of the analog [FIG. 8A]. This treatment did not differ from the joint treatment of the kinin agonist analog 1728 and SACC-0412064, the latter which had low antagonistic effect in the cell assay. This low antagonistic effect can perhaps explain that the effect of the kinin agonist analog 1728 plus SACC-0412064 treatment was intermediate between those of analog 1728 and the no treatment group, the latter from which was also not significantly different [FIG. 8A]. In contrast, the antagonists SACC-0412000 and -10412066 significantly reduced the mobility of the hindgut [FIG. 8A]. Although SACC-0412006 agonized the calcium response at 100 μmol L⁻¹, we did not observe any increase in the hindgut contraction rate in the hindgut contraction assay, supporting a potential off-target agonistic activity at high concentration on the mammalian cells only [FIGS. 7A and B]. Hindguts incubated with SACC-0412002 and the kinin agonist analog 1728 showed reduced contraction rate (<one-fold smaller), compared to a two-fold increase of contraction rate in solvent plus the kinin agonist analog 1728 group (P<0.0001) [FIG. 8B].

### 3.7 Blast Analyses of Kinin Receptors

The R. microplus kinin receptor (Boophilus microplus leucokinin-like peptide receptor mRNA: AF228521.1; protein: ADM47603.1) and the ortholog Ae. aegypti kinin receptor (mRNA: AY596453.1; protein: AAT95982.1), both cloned in our laboratory, have a 70% similarity in their transcript sequences and 57.93% similarity in their protein sequences. BLAST searches in NCBI using the R. microplus kinin receptor mRNA sequence (AF228521.1) as query, identified highly similar predicted mRNA sequences from other tick vector species. These top matching sequences are currently annotated as ‘Predicted RYamide receptor-like mRNAs’ from R. microplus (LOCI 19161208) (97.75% identity to the query sequence over a 98% query coverage), R. sanguineus (LOC 119395837) (9(0.44% identity over 58% coverage), Ixodes scapularis (LOC8039699) (82.16% identity over 39% coverage) and Dermacentor silvarum (LOC 119437389) (91.69% identity over 34% coverage). The alignment of these identified predicted receptor protein sequences revealed that R. sanguineus (XP_037518776.1) and Jr. Scapularis (XP 002413649.2) are 97.74% and 85.15% (respectively) identical to the R. microplus receptor query (ADM47603.1). For the predicted Dermacentor silvarum receptor (XP_037560349.1), the identity to the latter one is 97.45% over a query coverage of 69%. These results indicate that the currently annotated tick ‘RYamide receptor loci’ correspond to tick kinin receptor genes.

### 4. Discussion

In this study we aimed to identify novel small molecule ligands of the tick kinin receptor to expand our tool set towards elucidating the function of the tick kinin signaling system and identifying potential chemical leads for tick control. The kinin receptor from mosquito Ae. aegypti is activated by different kinin peptidomimetics that can induce different physiological functions, and specifically, the kinin agonist analog 1728 used in this study.^{16,36} This analog is antifeedant not only in adult Ae. aegypti but also in aphids,³⁷ and other kinin analogs are antifeedant in the kissing bug.³⁸ In addition, this mosquito receptor is activated by a series of analogs designed based on the endogenous mosquito kinins.²⁸ The tick kinin receptor is activated by kinin peptidomimetics that are more potent than 1728.²⁸ Further, we previously validated four small molecule antagonists of the human neurokinin receptors (the closest human GPCRs to invertebrate kinin receptors) as weak antagonists of the tick kinin receptor,¹⁹ suggesting that the search for more potent small molecule ligands was a promising approach. ‘Small molecules’ for therapeutic purposes are defined by the ‘Lipinski rule of five’, and include those <500 Da (oral administration).³⁹ however, this size rule is often violated for agrochemicals. In previous work we tested novel peptidomimetic ligands of the tick kinin receptor that were designed based on the sequences of endogenous tick kinins; all such mimetics were full agonists.²⁰ However, peptidomimetic ligands are more costly to synthesize and may have limited penetration through the arthropod cuticle, both of which limit their potential as agrochemicals. Lack of crystal structures of arthropod neuropeptide GPCRs remains a major obstacle for predicting small molecule ligands. The alternative approach to identify novel ligands of GPCRs is through high-throughput screening (HTS) of random small molecule libraries. Currently, only two published studies have performed HTS for novel ligand discovery on arthropod vector GPCR targets. A novel small molecule agonist of the mosquito neuropeptide Y (NPY) receptor was identified through HTS.⁴⁰ In addition, the invertebrate dopamine receptor, a biogenic amine GPCR, was subjected to HTS with target-specific libraries, and antagonists exhibiting considerable toxicity to mosquito larvae were discovered.⁴¹

The ‘dual-addition’ assay we developed here allows identification of agonist or antagonist in the same assay in a high-throughput mode.¹⁹ The theory of the ‘dual-addition’ assay is reviewed elsewhere.²⁵ Our targeted screening of the random small molecule library using the tick kinin receptor identified 18 antagonists after dose-response analyses of the initial 32 antagonist hits. The 18 validated antagonists comprised 0.09% of the total screened compounds. It is worth mentioning that the primary HTS and the secondary validation in the dose-response assays utilized different agonist peptides. The primary screen was done with an hexapeptide generic kinin, FFFSWGa, that although potent on the tick receptor,⁴² differed in structure from the tick endogenous kinins.¹⁹ This kinin analog was used because at the time the primary screen was performed, we had not yet identified and tested the R. microplus kinins. The implication is that the antagonist hits derived from the primary screen may have been biased towards this kinin agonist.

During validation of hits in the dose-response assays, we used one of the kinins from R. microplus, Rhimi-K-1, the sequence of which was deduced from cloning the peptide precursor, and its activity was verified on the tick kinin receptor.^{19,20} No agonist molecules were validated from the 26 putative agonist hits identified from high-throughput screening. This may be attributed to our rigorous standard for selecting agonists, requiring that a hit elicits a cellular response higher than 46% of the responses to 500 nmol L⁻¹ of the kinin peptide FFFSWGa; this concentration can elicit almost the maximal response (80% to 100%) from the recombinant tick kinin receptor cells.²⁸ The initial chosen threshold for selecting agonist and antagonist hits was 50% for both NPA and Iₒ. However, in order to obtain more hits from the performed primary screen, the data was manually curated, and two agonist hits and four antagonist hits that were slightly below the cut-off threshold were picked for dose-response validation, therefore, the actual cut-offs were 42% for antagonists and 46% for agonists.

The kinin agonist peptides have a conserved C-terminal motif, FX¹X²WGamide (X¹ and X² are variable residues).^{43,44} In contrast, the structure of the 18 potential antagonist small molecules had very low structural similarities among themselves (Suppl. Table 7). A structure-based in silico screen search resulted in identification of 27 putative antagonist hits. Dose-response validation experiments validated 40.7% of these predicted hits (11/27). Therefore, prediction of the ligand structure based on known antagonists can improve hit identification. In total we validated 36 antagonists of the tick kinin receptor that, based on their structural similarities, could be clustered into 27 groups (Suppl. Table 7). The larger cluster of molecules with high structural similarity included the eight lead analog molecules shown in FIG. 6. The structure-activity relationships analysis suggested that compounds with phenyl, naphthyl, or 4-Cl-phenyl at the Ar-position exhibited full antagonistic characteristics whereas substitution with four alternative groups resulted in decreased activities and loss of full antagonistic activity. Further receptor-ligand structure analyses followed by in silico design of hit molecules will be meaningful to improve the resolution of the SAR.

Although the dose-response assay validation in V/O cells allowed us to discard false agonist hits, the same cannot be concluded from the potential antagonist hits. Therefore, a functional assay was developed to verify the inhibitory effect of the most potent antagonists. For this, we reasoned the hindgut contractions had to be first chemically stimulated to verify the inhibitory activity of the small molecules. Kinin peptides were known as myokinins for their myotropic activity on the hindgut of various insect species.^{13,45} The hindgut bioassay has been the gold standard for evaluating the activity of myotropic or myoinhibitory ligands of GPCRs in insects and ticks.^{33,46} To determine the antagonistic activity of the validated compounds in a bioassay, we adapted the mosquito hindgut contraction assay we previously developed,³³ as a hindgut contraction inhibition assay. The equivalent assay was not attempted in a tick system because the exact physiological function of kinins in ticks remains unknown and we have not yet validated a tick muscle contraction assay in our laboratory. Our lab has developed parallel pipelines for studying kinins and kinin receptors from mosquitoes and ticks¹¹; both receptors were de-orphanized in our lab,^{20,31} and many kinin peptidomimetics tested side-by-side on both recombinant receptors showed comparable stimulatory activities on both receptors.^{21,28,42} Altogether, these results pointed to the mosquito hindgut contraction inhibition assay as a strong surrogate for testing the ligand activities of invertebrate kinin receptors.

In addition, the high amino acid sequence similarity among tick kinin receptors reported herein, added to our previous phylogenetic analysis of kinin peptide precursors that revealed high similarity in kinin sequences among tick species,¹⁹ support that the antagonists we discovered herein should also have activity on kinin receptors from other tick vector species.

Only one gene encoding the Ae. aegypti kinin receptor¹ (annotated as substance-K receptor, Gene ID: 5574266) can be found in the most recent genome of Ae. aegypti,⁴⁷ two predicted transcript variants⁴⁸ have identical protein coding region (AAEL006636-RA and RB on VectorBase²), therefore, there is only one form of the protein. Although the R. microplus genome is less well-annotated, there is also only one functional kinin receptor identified so far. Although the three endogenous mosquito kinins (aedeskinins) stimulate hindgut contractions in the mosquito Ae. aegypti at concentrations of 10⁻⁸ M,⁴⁹ and kinin peptides increase hindgut contractile activity in other insects such as cockroaches and kissing bug,^{13,45}, we chose the kinin agonist analog 1728 to stimulate the mosquito hindgut contraction before applying the antagonists in the hindgut contraction inhibition assay. The kinin agonist analog 1728 ([Aib]FF[Aib]WGa) was chosen because it is a positive control peptide with enhanced peptidase resistance, and is a very potent agonist on both mosquito and tick kinin recombinant receptors.²¹ This analog induces Rhodnius prolixus hindgut contraction at 10⁻¹⁶ M,⁴⁵ and it was also active in the diuretic assays with Drosophila melanogaster renal organs at 2.5×10⁻⁵ M but not active in the 10⁻⁷ M range to increase fluid secretion from the renal organs.⁵⁰ Likewise, in the current study the kinin agonist analog 1728 was active in the mosquito hindgut when tested at 10 μmol L⁻¹. It is worth noting that the increase of hindgut contraction in response to the kinin agonist analog 1728 had not been previously reported for the mosquito. The increase of hindgut contraction rate was more clearly observed under the microscope 1 h after the addition of the kinin agonist analog 1728, and also in tissues incubated with 1 μmol L⁻¹ of an endogenous mosquito kinin, aedeskinin 2 (NPFHAWGamide, data not shown). In contrast, kinins almost immediately increased hindgut contractions in the cockroach and kissing bug.^{13,45} This difference in observed speed of activity is likely due to the difference in the measurement methods as other researchers^{13,45} used electrodes to measure the movement of the hindgut, which are much more sensitive to small contractions, whereas we used video recording under the dissecting microscope and visual inspection by the same operator.

After validating the hindgut contractile activity of the kinin agonist analog 1728 in females of Ae. aegypti, we tested the most potent antagonist compounds that were commercially available in the hindgut contraction inhibition assay. All three molecules we tested inhibited the contraction of the hindgut. Indeed, the active compounds tested at 100 μmol L⁻¹ either partially or completely reduced the amplitude of the peristaltic contraction of the hindgut after incubation with the tissue for 1 h. Although these antagonists were able to inhibit the fluorescence response at 30 μmol L⁻¹ in the cellular assay with CHO-K1 cells (FIG. 7), we decided to use a higher concentration of 100 μmol L⁻¹ in the tissue assay to account for potential degradation by the tissues during the incubation period. Thus, the antagonists are likely active at lower concentrations. When we reduced the concentration of SACC-0412060 and SACC-0412066 to 10 μmol L⁻¹ in the hindgut bioassay, no increase in the hindgut contraction rate was observed 1 h after the kinin agonist analog 1728 was added (data not shown), whereas the hindgut contraction rate in the kinin agonist analog 1728 treatment increased by a factor of 2 (FIG. 8)). During the validation of small molecules in the dose-response fluorescence assay, there was the possibility that the small molecules themselves elicited the dose-dependent antagonistic activity by quenching the fluorescence response. However, the results from the hindgut contraction inhibition assay, independently from the cellular fluorescence calcium assay, confirmed that the small molecules were able to block the myotropic activity induced by the kinin agonist analog 1728, even when the latter was applied at 10 μmol L⁻¹ (FIG. 7). The potent antagonists (IC₅₀ s 1.2-3.2 μmol L⁻¹) identified from the in silico search of small molecules in the ChemBridge™ library were chosen for validation in the hindgut contraction inhibition assay because of their immediate commercial availability. However, the rest of the 11 potent full antagonists that had IC₅₀s from 0.67- to 8-μmol L⁻¹ may have greater or similar activity on this tissue than the assayed molecules, and therefore, remain to be tested.

In conclusion, this study utilized a HTS of a library of random small molecules combined with a structure-based in silico screen to identify novel ligands of the tick kinin receptor. We validated 36 potential antagonists on the recombinant receptor using a dual-addition cell assay we developed as part of this study. This is the first reported HTS of a neuropeptide GPCR on any tick species. Three of the most potent small molecules with similar structure were tested on the recombinant mosquito orthologous receptor and in the mosquito hindgut contraction inhibition assay. For the first time, the hindgut contractile activity of the kinin agonist analog 1728 was reported for mosquito. The antagonists identified in the HTS and validated in dose-response cellular assays also inhibited the myotropic effect of the potent kinin agonist analog 1728. These antagonists are tools to study arthropod kinin signaling physiology and are potentially useful for novel approaches for pest control. Bioassays with live ticks remain to be performed, as access to this quarantined species was limited by regulations imposed due to SARS-CoV-2 (COVID-19) pandemic.

### REFERENCES

1. Jongejan F and Uilenberg G, The global importance of ticks. Parasitology 129: S3-S14(2004).
2. Bock R E, Jackson, L. A., de Vos, A. J., and Jorgensen, W. K. Babesiosis of cattle. Parasitology. (2004). 129: S247-S269.
3. Rodriguez-Vivas R I, Jonsson N N and Bhushan C, Strategies for the control of Rhipicephalus microplus ticks in a world of conventional acaricide and macrocyclic lactone resistance. Parasilol Res 117: 3-29 (2018).
4. Angus B M, The history of the cattle tick Boophilus microplus in Australia and achievements in its control. Int J Parasitol 26: 1341-1355 (1996).
5. Madder M. Thys E, Achi L, Touré A and De Deken R, Rhipicephalus (Boophilus) microplus: a most successful invasive tick species in West-Africa. Exp App Acarol 53: 139-145 (2011).
6. Rodriguez-Vivas R I, Grisi L, de León AAP, Villela H S, de Jesus Torres-Acosta J F, Sanchez H F, et al., Potential economic impact assessment for cattle parasites in Mexico. Revista Mexican de Ciencias Pecuarias 8: 61-74 (2017).
7. Thomas D B, Klafke G, Busch J D, Olafson P U, Miller R A, Mosqueda J, et al., Tracking the increase of acaricide resistance in an invasive population of Cattle Fever Ticks (Acari: Ixodidae) and implementation of real-time PCR assays to rapidly genotype resistance mutations. Ann Entomol Soc Am 2020).
8. Hilger D, Masureel M and Kobilka B K, Structure and dynamics of GPCR signaling complexes. Nat Struct Mol Biol 25: 4-12 (2018).
9. Hauser A S, Attwood M M, Rask-Andersen M, Schiöth H B and Gloriam D E, Trends in GPCR drug discovery: new agents, targets and indications. Nat Rev Drug Discov 16: 829 (2017).
10. Kita T, Hayashi T, Ohtani T. Takao H, Takasu H, Liu G, et al., Amitraz and its metabolite differentially activate α- and β-adrenergic-like octopamine receptors. Pest Manag Sci 73: 984-990 (2017).
11. Pietrantonio P V, Xiong C, Nachman R J and Shen Y, G protein-coupled receptors in arthropod vectors: Omics and pharmacological approaches to elucidate ligand-receptor interactions and novel organismal functions. Curr Opin Insect Sci 29: 12-20 (2018).
12. Audslev N and Down R E, G protein coupled receptors as targets for next generation pesticides. Insect Biochem Molec 67: 27-37 (2015).
13. Holman G, Cook B and Nachman R, Isolation, primary structure and synthesis of two neuropeptides from Leucophaea maderae: members of a new family of cephalomyotropins. Comp Biochem Phys C 84: 205-211 (1986).
14. Kersch C N and Pietrantonio P V, Mosquito Aedes aegypti (L.) leucokinin receptor is critical for in vivo fluid excretion post blood feeding. FEBS Lett 585: 3507-3512 (2011).
15. Harshini S, Manchu V. Sunitha V. Sreekumar S and Nachman R, In vitro release of amylase by culekinins in two insects: Opsinia arenosella (Lepidoptera) and Rhynchophorus ferrugineus (Coleoptera). Trends in Life Sciences 17: 61-64 (2(03).
16. Kwon H, Agha M A, Smith R C, Nachman R J, Marion-Poll F and Pietrantonio P V, Leucokinin mimetic elicits aversive behavior in mosquito Aedes aegypti (L.) and inhibits the sugar taste neuron. Proc Nail Acad Sci 113: 6880-6885 (2016).
17. Kim D-H, Kim Y-J and Adams M E, Endocrine regulation of airway clearance in Drosophila. Proc Nail Acad Sci: 201717257 (2018).
18. Brock C M, Temeyer K B, Tidwell J, Yang Y, Blandon M A, Carreón-Camacho D, et al., The leucokinin-like peptide receptor from the cattle fever tick, Rhipicephalus microplus, is localized in the midgut periphery and receptor silencing with validated double-stranded RNAs causes a reproductive fitness cost. Int J Parasitol 49: 287-299 (2019).
19. Xiong C, Baker D and Pietrantonio P V, The cattle fever tick, Rhipicephalus microplus, as a model for forward pharmacology to elucidate kinin GPCR function in the Acari. Front Physiol 10: 1008 (2019).
20. Xiong C. Kaczmarek K, Zabrocki J, Nachman R J and Pietrantonio P V, Activity of native tick kinins and peptidomimetics on the cognate target G protein-coupled receptor from the cattle fever tick. Rhipicephalus microplus (Acari: Ixodidae). Pest Manag Sci (2020). 76: 3423-3431.
21. Taneja-Bageshwar S. Strey A, Isaac R E, Coast G M, Zubrzak P. Pietrantonio P V, et al., Biostable agonists that match or exceed activity of native insect kinins on recombinant arthropod GPCRs. Gen Comp Endocrinol 162: 122-128 (2009).
22. Thomsen W. Frazer J and Unett D, Functional assays for screening GPCR targets. Current opinion in biotechnology 16: 655-665 (2005).
23. Holmes S P, Barhoumi R, Nachman R J and Pietrantonio P V, Functional analysis of a G protein-coupled receptor from the Southern cattle tick Boophilus microplus (Acari: Ixodidae) identifies it as the first arthropod myokinin receptor. Insect Mol Biol 12: 27-38 (2003).
24. Lu H L, Kersch C N, Taneja-Bageshwar S and Pietrantonio P V, A calcium bioluminescence assay for functional analysis of mosquito (Aedes aegypti) and tick (Rhipicephalus microplus) G protein-coupled receptors. Jove-J Vis Exp 2011).
25. Ma Q, Ye L, Liu H, Shi Y and Zhou N, An overview of Ca2+mobilization assays in GPCR drug discovery. Expert Opin Drug Dis 12: 511-523 (2017).
26. Zhang J-H, Chung T D and Oldenburg K R, A simple statistical parameter for use in evaluation and validation of high throughput screening assays. J Biomo Screen 4: 67-73 (1999).
27. Blanchard J E, Elowe N H, Huitema C, Fortin P D, Cechetto J D, Eltis L D, et al., High-Throughput Screening Identifies Inhibitors of the SARS Coronavirus Main Proteinase. Chem Biol 11: 1445-1453 (2004).
28. Xiong C, Kaczmarek K. Zabrocki J, Pietrantonio P V and Nachman R J, Evaluation of Aib and PEG-polymer insect kinin analogs on mosquito and tick GPCRs identifies potent new pest management tools with potentially enhanced biostability and bioavailability. Gen Comp Endocrinol 278: 58-67 (2019).
29. Hohman M. Gregory K, Chibale K, Smith P J, Ekins S and Bunin B, Novel web-based tools combining chemistry informatics, biology and social networks for drug discovery. Drug discovery today 14: 261-270 (2009).
30. Aggarwal A, Parai M K, Shetty N, Wallis D, Woolhiser L, Hastings C, et al., Development of a novel lead that targets M. tuberculosis polyketide synthase 13. Cell 170: 249-259. e225 (2017).
31. Pietrantonio P V, Jagge C, Taneja-Bageshwar S. Nachman R J and Barhoumi R, The mosquito Aedes aegypti (L.) leucokinin receptor is a multiligand receptor for the three Aedes kinins. Insect Mol Biol 14: 55-67 (2005).
32. Jagge C L and Pietrantonio P V, Diuretic hormone 44 receptor in Malpighian tubules of the mosquito Aedes aegypti: evidence for transcriptional regulation paralleling urination. Insect Mol Biol 17: 413-426 (2008).
33. Kwon H and Pietrantonio P V, Calcitonin receptor 1 (AedaeGPCRCAL1) hindgut expression and direct role in myotropic action in females of the mosquito Aedes aegypn (L.). Insect Biochem Molec 43: 588-593 (2013).
34. Clark T M, Hayes T K, Holman G and Beyenbach K W, The concentration-dependence of CRF-like diuretic peptide: mechanisms of action. J Exp Biol 201: 1753-1762 (1998).
35. Odland G C and Jones J C, Contractions of the hindgut of adult Aedes aegypti with special reference to the development of a physiological saline. Ann Entomol Soc Am 68: 613-616 (1975).
36. Schepel S A, Fox A J, Miyauchi J T, Sou T, Yang J D, Lau K, et al., The single kinin receptor signals to separate and independent physiological pathways in Malpighian tubules of the yellow fever mosquito. Am J Physiol-Reg I 299: R612-R622 (2010).
37. Smagghe G, Mandian K, Zubrzak P and Nachman R J, Antifeedant activity and high mortality in the pea aphid Acyrthosiphon pisum (Hemiptera: Aphidae) induced by biostable insect kinin analogs. Peptides 31: 498-505 (2010).
38. Lange A B, Nachman R J, Kaczmarek K and Zabrocki J, Biostable insect kinin analogs reduce blood meal and disrupt ecdysis in the blood-gorging Chagas' disease vector, Rhodnius prolixus. Peptides 80: 108-113 (2016).
39. Lipinski C A, Rule of five in 2015 and beyond: Target and ligand structural limitations, ligand chemistry structure and drug discovery project decisions. Adv Drug Deliver Rev 101: 34-41 (2016).
40. Duvall L B, Ramos-Espiritu L, Barsoum K E, Glickman J F and Vosshall L B, Small-molecule agonists of Ae. aegypti neuropeptide Y receptor block mosquito biting. Cell 176: 687-701. e685 (2019).
41. Ejendal K F K, Meyer J M, Brust T F, Avramova L V, Hill C A and Watts V J, Discovery of antagonists of tick dopamine receptors via chemical library screening and comparative pharmacological analyses. Insect Biochem Molec 42: 846-853 (2012).
42. Taneja-Bageshwar S, Strey A, Zubrzak P. Pietrantonio P V and Nachman R J, Comparative structure-activity analysis of insect kinin core analogs on recombinant kinin receptors from Southern cattle tick Boophilus microplus (Acari: Ixodidae) and mosquito Aedes aegypti (Diptera: Culicidae). Arch Insect Biochem 62: 128-140 (2006).
43. Nachman R J and Holman G M. Myotropic Insect Neuropeptide Families from the Cockroach Leucophaea maderae: Structure-Activity Relationships. In Insect neuropeptides: chemistry, biology, and action, ed. by Menn J J, Masler, Edward P. American Chemical Society: Washington, D. C., pp. 194-214 (1991).
44. Nachman R J, Strev A, Isaac E, Pryor N, Lopez J D, Deng J-G, et al., Enhanced in vivo activity of peptidase-resistant analogs of the insect kinin neuropeptide family. Peptides 23: 735-745 (2002).
45. Bhatt G, da Silva R, Nachman R J and Orchard I, The molecular characterization of the kinin transcript and the physiological effects of kinins in the blood-gorging insect, Rhodnius prolixus. Peptides 53: 148-158 (2014).
46. Šimo L and Park Y, Neuropeptidergic control of the hindgut in the black-legged tick Ixodes scapularis. Int J Parasitol 44: 819-826 (2014).
47. Matthews B J, Dudchenko O, Kingan S B, Koren S, Antoshechkin I, Crawford J E, et al., Improved reference genome of Aedes aegypti informs arbovirus vector control. Nature 563: 501-507 (2018).
48. Matthews B J, McBride C S, DeGennaro M, Despo O and Vosshall L B, The neurotranscriptome of the Aedes aegypti mosquito. BMC genomics 17: 1-20 (2016).
49. Veenstra J A, Pattillo J M and Petzel D H, A single cDNA encodes all three aedesleucokinins, which stimulate both fluid secretion by the malpighian tubules and hindgut contractions. J Biol Chem 272: 10402-10407 (1997).
50. Alford L, Marley R, Dornan A. Dow J A, Nachman R J and Davies S A, Desiccation, thermal stress and associated mortality in Drosophila fruit flies induced by neuropeptide analogue treatment. Journal of Pest Science 92: 1123-1137 (2019).

TABLE 1
Summary of the assay quality of each screen. The * indicates a Z′-factor could not be calculated due to pulled data from two plates.

| | | | | | | | | | | Antagonist | Agonist |
|---|---|---|---|---|---|---|---|---|---|---|---|
| | μₛ | σₛ | | | μₛ | σₛ | | | Z′- | threshold | threshold |
| Plate name | (compound) | (compound) | μ_{c-} | σ_{c-} | (peptide) | (peptide) | μ_{c+} | σ_{c+} | factor | (50% μ_{c+}) | (μ_{c+}) |
| Library (2019) each | n = 640 | n = 640 | n = 128 | n = 128 | n = 640 | n = 640 | n = 128 | n = 128 | | | |
| library was screened | | | | | | | | | | | |
| in duplicate | | | | | | | | | | | |
| SAC2-19-6095 | 3,070 | 4,005 | 1,971 | 1,033 | 34,174 | 10,869 | 33,587 | 7,252 | * | 16,794 | 33,587 |
| SAC2-20-6100 | 1,485 | 944 | 994 | 854 | 25,802 | 9,088 | 22,250 | 7,859 | | 11,125 | 22,250 |
| SAC2-21-6105 | 1,616 | 2,735 | 1,088 | 652 | 28,688 | 6,522 | 27,842 | 8,117 | | 13,921 | 27,842 |
| SAC2-96-6480 | 3,654 | 1,713 | 1,839 | 776 | 46,141 | 5,499 | 46,373 | 6,376 | | 23,187 | 46,373 |
| SAC2-97-6485 | 2,492 | 1,424 | 1,685 | 928 | 54,254 | 11,935 | 63,993 | 6,945 | | 31,997 | 63,993 |
| Library (2019) | n = 320 | n = 320 | n = 64 | n = 64 | n = 320 | n = 320 | n = 64 | n = 64 | | | |
| SAC2-100-6500 | 1,693 | 1,085 | 812 | 776 | 30,320 | 3,740 | 33,884 | 3,198 | 0.6 | 16,942 | 33,884 |
| SAC2-101-6505 | 2,789 | 1,273 | 1,463 | 710 | 31,136 | 3,487 | 30,126 | 3,003 | 0.6 | 15,063 | 30,126 |
| SAC2-156-6780 | 2,555 | 1,157 | 1,395 | 742 | 36,484 | 4,154 | 36,902 | 4,113 | 0.6 | 18,451 | 36,902 |
| SAC2-157-6785 | 2,079 | 1,319 | 1,385 | 2,315 | 37,304 | 6,452 | 38,352 | 3,817 | 0.5 | 19,176 | 38,352 |
| SAC2-158-6790 | 3,500 | 2,912 | 1,373 | 1,062 | 52,986 | 8,981 | 55,643 | 7,710 | 0.5 | 27,822 | 55,643 |
| SAC2-159-6795 | 3,842 | 1,729 | 2,218 | 1,341 | 56,110 | 8,629 | 57,429 | 8,067 | 0.5 | 28,715 | 57,429 |
| SAC2-22-6110 | 1,494 | 1,003 | 829 | 718 | 32,782 | 4,889 | 32,026 | 5,641 | 0.4 | 16,013 | 32,026 |
| SAC2-3-6015 | 1,317 | 1,796 | 1,042 | 838 | 25,852 | 3,977 | 28,005 | 3,910 | 0.5 | 14,003 | 28,005 |
| SAC2-4-6020 | 2,070 | 2,321 | 1,746 | 768 | 26,407 | 4,485 | 27,741 | 3,647 | 0.5 | 13,871 | 27,741 |
| SAC2-5-6025 | 2,030 | 1,139 | 1,659 | 595 | 30,050 | 5,194 | 30,216 | 3,714 | 0.5 | 15,108 | 30,216 |
| SAC2-6-6030 | 2,385 | 1,578 | 2,319 | 971 | 24,938 | 5,532 | 29,166 | 3,608 | 0.5 | 14,583 | 29,166 |
| SAC2-7-6035 | 6,301 | 5,489 | 4,431 | 2,208 | 46,873 | 5,374 | 48,845 | 5,132 | 0.5 | 24,423 | 48,845 |
| SAC2-8-6040 | 6,519 | 2,890 | 4,709 | 1,942 | 45,945 | 6,526 | 47,840 | 7,175 | 0.4 | 23,920 | 47,840 |
| SAC2-9-6045 | 6,532 | 3,144 | 4,550 | 2,176 | 54,751 | 7,443 | 56,396 | 7,146 | 0.5 | 28,198 | 56,396 |
| SAC2-10-6050 | 6,910 | 12,193 | 4,546 | 1,943 | 47,941 | 12,402 | 53,424 | 5,791 | 0.5 | 26,712 | 53,424 |
| SAC2-11-6055 | 3,763 | 3,446 | 1,381 | 839 | 53,439 | 6,184 | 57,839 | 6,755 | 0.6 | 28,920 | 57,839 |
| SAC2-12-6060 | 1,894 | 1,472 | 1,023 | 547 | 27,128 | 6,646 | 29,636 | 4,582 | 0.5 | 14,818 | 29,636 |
| SAC2-13-6065 | 4,038 | 2,702 | 3,893 | 1,491 | 63,017 | 6,569 | 71,331 | 5,880 | 0.7 | 35,666 | 71,331 |
| SAC2-14-6070 | 3,841 | 1,601 | 3,583 | 1,449 | 66,481 | 7,368 | 70,005 | 7,875 | 0.6 | 35,003 | 70,005 |
| SAC2-15-6075 | 4,415 | 3,440 | 3,515 | 1,809 | 71,599 | 9,942 | 78,163 | 9,662 | 0.5 | 39,082 | 78,163 |
| SAC2-16-6080 | 2,786 | 3,558 | 2,291 | 1,881 | 51,583 | 7,127 | 55,627 | 6,456 | 0.5 | 27,814 | 55,627 |
| SAC2-17-6085 | 4,069 | 1,489 | 2,758 | 916 | 49,894 | 5,655 | 50,037 | 5,425 | 0.6 | 25,019 | 50,037 |
| SAC2-18-6090 | 4,369 | 4,808 | 2,920 | 1,113 | 56,462 | 7,450 | 59,939 | 8,388 | 0.5 | 29,970 | 59,939 |
| SAC2-23-6115 | 4,480 | 2,277 | 3,579 | 989 | 54,010 | 7,772 | 59,344 | 6,652 | 0.6 | 29,672 | 59,344 |
| SAC2-21-6105 | 2,425 | 1,695 | 2,036 | 806 | 41,485 | 3,556 | 41,577 | 2,908 | 0.7 | 20,788 | 41,577 |
| SAC2-42-6210 | 1,441 | 675 | 1,339 | 513 | 40,283 | 4,528 | 41,306 | 5,088 | 0.6 | 20,653 | 41,306 |
| SAC2-43-6215 | 2,019 | 719 | 1,637 | 448 | 40,686 | 4,153 | 37,867 | 3,952 | 0.6 | 18,934 | 37,867 |
| SAC2-44-6220 | Undetermined | | | | 34,009 | 3,649 | 33,751 | 3,629 | | 16,876 | 33,751 |
| SAC2-24-6120 | 4,793 | 2,656 | 4,304 | 2,068 | 83,429 | 6,562 | 88,398 | 6,124 | 0.7 | 44,199 | 88,398 |
| SAC2-25-6125 | 4,218 | 1,889 | 2,999 | 1,110 | 75,980 | 5,547 | 75,571 | 5,334 | 0.7 | 37,786 | 75,571 |
| SAC2-26-6130 | 2,810 | 1,354 | 2,217 | 873 | 34,396 | 3,483 | 34,482 | 3,593 | 0.6 | 17,241 | 34,482 |
| SAC2-27-6135 | 2,591 | 1,593 | 1,564 | 635 | 30,003 | 4,862 | 31,372 | 3,765 | 0.6 | 15,686 | 31,372 |
| SAC2-28-6140 | 2,861 | 1,382 | 2,450 | 990 | 32,213 | 6,036 | 32,020 | 3,945 | 0.5 | 16,010 | 32,020 |
| SAC2-29-6145 | 2,048 | 928 | 1,556 | 531 | 33,520 | 3,726 | 34,718 | 3,654 | 0.6 | 17,359 | 34,718 |
| SAC2-30-6150 | 1,621 | 688 | 1,449 | 532 | 23,287 | 2,500 | 22,849 | 2,790 | 0.5 | 11,425 | 22,849 |
| SAC2-31-6155 | 1,090 | 745 | 999 | 478 | 17,492 | 2,168 | 18,530 | 2,536 | 0.5 | 9,265 | 18,530 |
| SAC2-32-6160 | 1,280 | 808 | 1,029 | 523 | 20,135 | 2,291 | 19,581 | 2,092 | 0.6 | 9,791 | 19,581 |
| SAC2-33-6165 | 1,090 | 745 | 1,000 | 478 | Undetermined | | | | | 0 | 0 |
| SAC2-34-6170 | 598 | 1,379 | 785 | 1,270 | 34,044 | 6,127 | 32,375 | 2,377 | 0.7 | 16,188 | 32,375 |
| SAC2-35-6175 | 3,066 | 1,042 | 1,912 | 701 | 29,308 | 3,080 | 26,543 | 3,351 | 0.5 | 13,272 | 26,543 |
| SAC2-36-6180 | 7,426 | 14,691 | 3,546 | 767 | 37,020 | 12,363 | 32,489 | 3,085 | 0.6 | 16,245 | 32,489 |
| SAC2-37-6185 | 1,474 | 770 | 891 | 425 | 15,699 | 2,630 | 17,652 | 1,616 | 0.6 | 8,826 | 17,652 |
| SAC2-38-6190 | 1,786 | 784 | 1,134 | 466 | 16,582 | 1,639 | 16,915 | 1,706 | 0.6 | 8,458 | 16,582 |
| SAC2-39-6195 | 1,225 | 552 | 957 | 337 | 11,715 | 1,764 | 11,895 | 1,427 | 0.5 | 5,948 | 11,715 |
| SAC2-40-6200 | 1,379 | 470 | 1,058 | 309 | 9,412 | 1,354 | 9,627 | 946 | 0.6 | 4,814 | 9,412 |
| SAC2-41-6205 | 2,373 | 1,070 | 1,731 | 452 | 10,887 | 2,674 | 12,065 | 1,320 | 0.5 | 6,033 | 10,887 |
| SAC2-45-6225 | 1,140 | 545 | 944 | 478 | 18,244 | 1,804 | 16,765 | 1,593 | 0.6 | 8,383 | 18,244 |
| SAC2-46-6230 | 1,621 | 890 | 1,114 | 418 | 17,263 | 1,900 | 17,589 | 1,999 | 0.6 | 8,795 | 17,263 |
| SAC2-47-6235 | 1,451 | 760 | 754 | 514 | 22,127 | 2,922 | 20,337 | 2,517 | 0.5 | 10,169 | 22,127 |
| SAC2-48-6240 | 2,000 | 956 | 1,127 | 414 | 22,449 | 2,722 | 19,397 | 2,507 | 0.5 | 9,699 | 22,449 |
| SAC2-34-6170 | 3,650 | 8,594 | 3,277 | 1,286 | 60,032 | 6,448 | 59,188 | 4,915 | 0.7 | 29,594 | 60,032 |
| SAC2-36-6180 | 3,391 | 13,015 | 2,527 | 813 | 48,792 | 11,678 | 51,556 | 4,133 | 0.7 | 25,778 | 48,792 |
| SAC2-49-6245 | 1,965 | 796 | 1,254 | 528 | 44,959 | 3,495 | 41,449 | 3,445 | 0.7 | 20,725 | 44,959 |
| SAC2-50-6250 | 2,444 | 1,108 | 1,578 | 521 | 41,376 | 4,054 | 39,022 | 5,253 | 0.5 | 19,511 | 41,376 |
| SAC2-51-6255 | 1,002 | 521 | 553 | 478 | 18,797 | 2,149 | 19,910 | 1,358 | 0.7 | 9,955 | 18,797 |
| SAC2-52-6260 | 1,673 | 955 | 688 | 447 | 16,004 | 1,970 | 15,262 | 2,120 | 0.5 | 7,631 | 16,004 |
| SAC2-53-6265 | 1,286 | 598 | 759 | 415 | 21,340 | 2,047 | 19,150 | 2,090 | 0.6 | 9,575 | 21,340 |
| SAC2-54-6270 | 1,615 | 671 | 1,276 | 491 | 21,133 | 2,011 | 19,099 | 2,128 | 0.6 | 9,550 | 21,133 |

TABLE 2
List of hit molecules from the primary high-throughput screening summarized in Table 1. The ** indicates
compounds which were previously identified from the screening 960 compounds from SAC1 library.

| 384wp: screening drug plate | | 96wp: Master plate | | | | agonist | antagonist |
|---|---|---|---|---|---|---|---|
| Plate | Well | Plate | Well | Molecule | Hits | NPA (%) | Io (%) |
| SAC2-36- | J04 | SAC2vendor_EN: EN103276- | E02 | SACC-0129990 | agonist | 481 | |
| 6180 | | 195-8568 | | | | | |
| SAC2-10- | N05 | SAC2vendor_BAS: 46-8144 | G03 | SACC-0072619 | agonist | 446 | |
| 6050 | | | | | | | |
| SAC2-21- | F20 | SAC2vendor_EN: EN103276- | C10 | SACC-0052578 | agonist | 166 | |
| 6105 | | 002-8328 | | | | | |
| SAC2-34- | B03 | SAC2vendor_EN: EN103276- | A02 | SACC-0114394 | agonist | 152 | |
| 6170 | | 160-8528 | | | | | |
| SAC2-34- | O06 | SAC2vendor_EN: EN103276- | H03 | SACC-0095102 | agonist | 145 | |
| 6170 | | 189-8432 | | | | | |
| SAC2-19- | P18 | SAC2vendor_BAS: 47-8296 | H09 | SACC-0075838 | agonist | 143 | |
| 6095 | | | | | | | |
| SAC2-34- | C15 | SAC2vendor_EN: EN103276- | B08 | SACC-0101626 | agonist | 141 | |
| 6170 | | 178-8524 | | | | | |
| SAC2-19- | A19 | SAC2vendor_BAS: 57-8284 | A10 | SACC-0126930 | agonist | 133 | |
| 6095 | | | | | | | |
| SAC2-18- | J20 | SAC2vendor_BAS: 68-8280 | E10 | SACC-0055093 | agonist | 128 | |
| 6090 | | | | | | | |
| SAC2-7- | L11 | SAC2vendor_BAS: 19-8096 | F06 | SACC-0117741 | agonist | 127 | |
| 6035 | | | | | | | |
| SAC2-7- | N14 | SAC2vendor_BAS: 14-8104 | G07 | SACC-0026915 | agonist | 111 | |
| 6035 | | | | | | | |
| SAC2-41- | O06 | SAC2vendor_EN: EN103276- | H03 | SACC-0005314 | agonist | 108 | |
| 6205 | | 018-8644 | | | | | |
| SAC2-16- | C17 | SAC2vendor_BAS: 38-8236 | B09 | SACC-0060049 | agonist | 104 | |
| 6080 | | | | | | | |
| SAC2-4- | N13 | SAC2vendor_BAS: 15-8048 | G07 | SACC-0004441 | agonist | 90 | |
| 6020 | | | | | | | |
| SAC2-11- | I22 | SAC2vendor_BAS: 17-8164 | E11 | SACC-0128437 | agonist | 86 | |
| 6055 | | | | | | | |
| SAC2-6- | D18 | SAC2vendor_BAS: 4-8088 | B09 | SACC-0027895 | agonist | 84 | |
| 6030 | | | | | | | |
| SAC2-4- | D12 | SAC2vendor_BAS: 78-8056 | B06 | SACC-0040154 | agonist | 81 | |
| 6020 | | | | | | | |
| SAC2-3- | A20 | SAC2vendor_BAS: 10-8036 | A10 | SACC-0055903 | agonist | 75 | |
| 6015 | | | | | | | |
| SAC2-15- | I16 | SAC2vendor_BAS: 73-8228 | E08 | SACC-0095111 | agonist | 71 | |
| 6075 | | | | | | | |
| SAC2-27- | F08 | SAC2vendor_EN: EN103276- | C04 | SACC-0029825 | agonist | 70 | |
| 6135 | | 153-8424 | | | | | |
| SAC2-7- | L10 | SAC2vendor_BAS: 14-8104 | F05 | SACC-0015467 | agonist | 70 | |
| 6035 | | | | | | | |
| SAC2-46- | K12 | SAC2vendor_EN: EN103276- | F06 | SACC-0128771 | agonist | 69 | |
| 6230 | | 051-8724 | | | | | |
| SAC2-32- | P18 | SAC2vendor_EN: EN103276- | H09 | SACC-0062945 | agonist | 61 | |
| 6160 | | 019-8504 | | | | | |
| SAC2-34- | E19 | SAC2vendor_EN: EN103276- | C10 | SACC-0033336 | agonist | 51 | |
| 6170 | | 178-8524 | | | | | |
| SAC2-48- | H03 | SAC2vendor_EN: EN103276- | D02 | SACC-0024181 | agonist | 48 | |
| 6240 | | 194-8752 | | | | | |
| SAC2-39- | I03 | SAC2vendor_EN: EN103276- | E02 | SACC-0095062 | agonist | 47 | |
| 6195 | | 159-8604 | | | | | |
| SAC2-100- | A06 | SAC2vendor_LC: 0073-9572 | A03 | SACC-0115325 | antagonist | | 83 |
| 6500 | | | | | | | |
| SAC2-4- | I14 | SAC2vendor_BAS: 27-8052 | E07 | SACC-0064443 | antagonist | | 83 |
| 6020 | | | | | | | |
| SAC2-28- | H18 | SAC2vendor_EN: EN103276- | D09 | SACC-0063031 | antagonist | | 80 |
| 6140 | | 201-8440 | | | | | |
| SAC2-28- | I18 | SAC2vendor_EN: EN103276- | E09 | SACC-0064624 | antagonist | | 76 |
| 6140 | | 016-8436 | | | | | |
| SAC2-34- | K04 | SAC2vendor_EN: EN103276- | F02 | SACC-0081272 | antagonist | | 74 |
| 6170 | | 189-8432 | | | | | |
| SAC2-37- | K14 | SAC2vendor_EN: EN103276- | F07 | SACC-0057260 | antagonist | | 73 |
| 6185 | | 017-8580 | | | | | |
| SAC2-101- | G17 | SAC2vendor_LC: 0092-9580 | D09 | SACC-0105544 | antagonist | | 72 |
| 6505 | | | | | | | |
| SAC2-23- | E19 | SAC2vendor_EN: EN103276- | C10 | SACC-0024648 | antagonist | | 70 |
| 6115 | | 009-8348 | | | | | |
| SAC2-13- | E19 | SAC2vendor_BAS: 25-8188 | C10 | SACC-0053274 | antagonist | | 68 |
| 6065 | | | | | | | |
| SAC2-156- | E07 | SAC2vendor_MB P: 36-10460 | C04 | SACC-0026188 | antagonist | | 67 |
| 6780 | | | | | | | |
| SAC2-21- | L12 | SAC2vendor_EN: EN103276- | F06 | SACC-0048694 | antagonist | | 65 |
| 6105 | | 002-8328 | | | | | |
| SAC2-157- | F21 | SAC2vendor_MB_P: 2-10480 | C11 | SACC-0033457 | antagonist | | 63 |
| 6785 | | | | | | | |
| SAC2-22- | O06 | SAC2vendor_EN: EN103276- | H03 | SACC-0037245 | antagonist | | 63 |
| 6110 | | 020-8340 | | | | | |
| SAC2-158- | F03 | SAC2vendor_SYN: 33-10496 | C02 | SACC-0007370 | antagonist | | 60 |
| 6790 | | | | | | | |
| SAC2-27- | K08 | SAC2vendor_EN: EN103276- | F04 | SACC-0018618 | antagonist | | 58 |
| 6135 | | 031-8420 | | | | | |
| SAC2-47- | M03 | SAC2vendor_EN: EN103276- | G02 | SACC-0088454 | antagonist | | 57 |
| 6235 | | 129-8732 | | | | | |
| SAC2-158- | I11 | SAC2vendor_SYN: 42-10492 | E06 | SACC-0048722 | antagonist | | 56 |
| 6790 | | | | | | | |
| SAC2-6- | O15 | SAC2vendor_BAS: 3-8076 | H08 | SACC-0068485 | antagonist | | 56 |
| 6030 | | | | | | | |
| SAC2-46- | K15 | SAC2vendor_EN: EN103276- | F08 | SACC-0101074 | antagonist | | 55 |
| 6230 | | 023-8716 | | | | | |
| SAC2-5- | F16 | SAC2vendor_BAS: 20-8072 | C08 | SACC-0006795 | antagonist | | 55 |
| 6025 | | | | | | | |
| SAC2-15- | H14 | SAC2vendor_BAS: 29-8232 | D07 | SACC-0048555 | antagonist | | 54 |
| 6075 | | | | | | | |
| SAC2-30- | F22 | SAC2vendor_EN: EN103276- | C11 | SACC-0108986 | antagonist | | 53 |
| 6150 | | 014-8472 | | | | | |
| SAC2-4- | B03 | SAC2vendor_BAS: 15-8048 | A02 | SACC-0037442 | antagonist | | 52 |
| 6020 | | | | | | | |
| SAC2-25- | A12 | SAC2vendor_EN: EN103276- | A06 | SACC-0125713 | antagonist | | 47 |
| 6125 | | 015-8388 | | | | | |
| SAC2-31- | O09 | SAC2vendor_EN: EN103276- | H05 | SACC-0027607 | antagonist | | 47 |
| 6155 | | 139-8476 | | | | | |
| SAC2-26- | K08 | SAC2vendor_EN: EN103276- | F04 | SACC-0131907 | antagonist | | 44 |
| 6130 | | 120-8404 | | | | | |
| SAC2-35- | L13 | SAC2vendor_EN: EN103276- | F07 | SACC-0025692 | antagonist | | 42 |
| 6175 | | 078-8544 | | | SACC-0126875 | antagonist | ** |
| | | | | | SACC-0121252 | antagonist | ** |
| | | | | | SACC-0113072 | antagonist | ** |
| | | | | | SACC-0050177 | antagonist | ** |
| | | | | | SACC-0010666 | antagonist | ** |

TABLE 3
Validation of 58 hit molecules in the dose-dependent assay. Dose-response curves (exemplary data presented
in FIGS. 4A-4K) were generated from the fluorescence responses of the recombinant tick kinin receptor (BMLK3)
and vector-only cell (V/O) in the dual-addition assay (described in Example 1 at the Materials and Method).
The Relative Fluorescence Units (RFU) in the Y-axis was normalized by subtracting the background signal.

| Molecule Name | BMLK3 EC50 (μM) | BMLK3 IC50 (μM) | Vector only EC50 (μM) | V/O antagonist IC50 (μM) | |
|---|---|---|---|---|---|
| SACC-0131907 | >25.0 | >25.0 | <0.0285 | 1.19 | *** |
| SACC-0129990 | 13.9 | 0.282 | 15.3 | 0.304 | *** |
| SACC-0128771 | 4.98 | 5.49 | 5.59 | 5.98 | *** |
| SACC-0128437 | 1.13 | 1.4 | 1.5 | 2.37 | *** |
| SACC-0126930 | <0.0285 | >25.0 | 0.761 | <0.0285 | *** |
| SACC-0125713 | 12.1 | 4.95 | 0.0558 | 0.462 | ✓ |
| SACC-0117741 | 1.74 | <0.0285 | 1.89 | 1.49 | *** |
| SACC-0115325 | 0.299 | >25.0 | >25.0 | >25.0 | *** |
| SACC-0114394 | 12 | 10.9 | 4.19 | 4.32 | *** |
| SACC-0108986 | 0.684 | 0.363 | 0.132 | 3.87 | *** |
| SACC-0105544 | <0.0285 | 3.33 | 11.4 | 0.816 | ✓ |
| SACC-0101626 | <0.0285 | 2.02 | <0.0285 | >25.0 | *** |
| SACC-0101074 | 11 | 10.9 | 2.58 | 2.95 | ✓ |
| SACC-0095111 | >25.0 | 3.32 | >25.0 | 7.33 | *** |
| SACC-0095102 | 0.974 | 1.88 | <0.0285 | 2.21 | *** |
| SACC-0095062 | >25.0 | >25.0 | >25.0 | <0.0285 | *** |
| SACC-0088454 | >25.0 | >25.0 | >25.0 | 14.4 | *** |
| SACC-0081272 | 1.48 | <0.0285 | 0.0485 | >25.0 | *** |
| SACC-0075838 | 4.37 | 0.0845 | >25.0 | >25.0 | *** |
| SACC-0072619 | 1.22 | 1.02 | 1.07 | 1.06 | *** |
| SACC-0068485 | 0.743 | 4.15 | 3.77 | 0.549 | *** |
| SACC-0064624 | 0.504 | 0.21 | >25.0 | >25.0 | *** |
| SACC-0064443 | 2.01 | 0.666 | 9.13 | >25.0 | ✓ |
| SACC-0063031 | 0.246 | 0.242 | 17 | 4.45 | *** |
| SACC-0062945 | <0.0285 | 0.128 | 0.0604 | 3.84 | *** |
| SACC-0060049 | 4.55 | >25.0 | 1.93 | 0.243 | *** |
| SACC-0057260 | 0.54 | 6.63 | 1.05 | >25.0 | ✓ |
| SACC-0055903 | 6.76 | 6.02 | 3.68 | 2.63 | *** |
| SACC-0055093 | 0.898 | 10.3 | 0.683 | 0.376 | *** |
| SACC-0053274 | 0.808 | 1.28 | 0.849 | 4.28 | ✓ |
| SACC-0052578 | >25.0 | >25.0 | >25.0 | >25.0 | *** |
| SACC-0048722 | 4.17 | >25.0 | <0.0285 | 4.32 | *** |
| SACC-0048694 | 0.281 | >25.0 | 6.87 | 17.5 | *** |
| SACC-0048555 | 8.88 | 1.51 | 12.8 | 3.59 | ✓ |
| SACC-0040154 | 13.2 | 3.09 | 0.12 | 1.24 | *** |
| SACC-0037442 | 7.77 | <0.0285 | >25.0 | 7.18 | *** |
| SACC-0037245 | 5.78 | 4.43 | <0.0285 | 0.605 | *** |
| SACC-0033457 | 0.122 | 14.8 | 11.5 | 0.679 | ✓ |
| SACC-0033336 | 2.95 | 7.17 | 4.34 | >25.0 | *** |
| SACC-0029825 | 6.95 | <0.0285 | 5.46 | 9.81 | *** |
| SACC-0027895 | >25.0 | >25.0 | >25.0 | >25.0 | *** |
| SACC-0027607 | <0.0285 | 0.483 | <0.0285 | >25.0 | ✓ |
| SACC-0026915 | 0.662 | 4.6 | 1 | >25.0 | *** |
| SACC-0026188 | <0.0285 | 0.225 | 0.154 | >25.0 | *** |
| SACC-0025692 | >25.0 | 0.765 | 9.54 | 0.112 | *** |
| SACC-0024648 | 0.346 | 2.11 | 0.665 | 6.82 | ✓ |
| SACC-0024181 | 9.01 | 0.252 | 8.1 | 6.27 | *** |
| SACC-0018618 | >25.0 | 4.29 | >25.0 | >25.0 | ✓ |
| SACC-0015467 | 4.32 | 6.3 | 3.45 | 7.1 | *** |
| SACC-0007370 | 1.78 | 1.42 | <0.0285 | >25.0 | *** |
| SACC-0006795 | >25.0 | 17.3 | <0.0285 | >25.0 | ✓ |
| SACC-0005314 | 0.956 | 3.09 | 0.474 | 0.434 | ✓ |
| SACC-0004441 | 3.5 | 1.72 | 3.51 | 3.05 | *** |
| SACC-0126875 | 5.73 | 8.51 | 4.61 | >25.0 | ✓ |
| SACC-0121252 | 0.177 | >25.0 | >25.0 | 2.53 | ✓ |
| SACC-0113072 | 2.79 | 2.33 | 1.95 | 2.59 | ✓ |
| SACC-0050177 | 7.08 | >25.0 | >25.0 | 12.3 | ✓ |
| SACC-0010666 | 0.914 | 0.808 | 1.35 | 14.9 | ✓ |

A *** indicates molecules which are either off-target or of low activity (<25 μM). A ✓ indicates molecules validated with Rhimi-K-1 (Pass or Fail).

TABLE 4
Validation of 27 virtual hit molecules in the dose-dependent assay. Dose-response curves (exemplary data presented in FIGS.
4A-4K) were generated from the fluorescence responses of the recombinant tick kinin receptor (BMLK3) and vector-only cell
(V/O) in the dual-addition assay (described in the Materials and Method). The Relative Fluorescence Units (RFU) in the Y-
axis was normalized by subtracting the background signal. Molecules with a check in column K were validated as antagonists.

| Molecule Name | BMLK3 EC50 (μM) | BMLK3 antagonist IC50 (μM) | Vector only EC50 (μM) | V/O antagonist IC50 (μM) | |
|---|---|---|---|---|---|
| SACC-0131532 | 16.6 | 1.41 | >25.0 | >25.0 | |
| SACC-0125715 | >25.0 | 1.55 | 14.3 | 0.711 | ✓ |
| SACC-0123851 | 6.57 | 15.3 | 6.85 | 2.36 | ✓ |
| SACC-0117388 | 0.124 | 13.2 | 1.78 | 8.39 | |
| SACC-0112961 | >25.0 | <0.0285 | 0.243 | >25.0 | |
| SACC-0099442 | 5.4 | 7.17 | 10.8 | 1.12 | ✓ |
| SACC-0095849 | >25.0 | 8.47 | 0.393 | 0.914 | |
| SACC-0089495 | 3.04 | 6.53 | 9.06 | 5.99 | ✓ |
| SACC-0087120 | 1.11 | <0.0285 | 3.16 | >25.0 | |
| SACC-0072383 | 0.326 | 0.501 | 0.294 | >25.0 | |
| SACC-0064924 | 17.3 | <0.0285 | 11.9 | >25.0 | |
| SACC-0058222 | >25.0 | 15.4 | >25.0 | 11.8 | ✓ |
| SACC-0054132 | <0.0285 | 3.69 | 2.56 | 0.207 | ✓ |
| SACC-0047526 | >25.0 | >25.0 | >25.0 | >25.0 | |
| SACC-0046099 | >25.0 | 8.31 | 3.58 | 2.45 | |
| SACC-0039825 | <0.0285 | 8.06 | >25.0 | 0.334 | |
| SACC-0039590 | 2.27 | >25.0 | 1.63 | 5.58 | ✓ |
| SACC-0034373 | 1.96 | 6.22 | 2.49 | >25.0 | ✓ |
| SACC-0029249 | >25.0 | 1.88 | >25.0 | <0.0285 | |
| SACC-0029037 | 4.1 | >25.0 | 12.4 | 7.67 | ✓ |
| SACC-0025777 | >25.0 | 0.106 | 1.57 | >25.0 | |
| SACC-0021424 | >25.0 | 13.6 | <0.0285 | >25.0 | ✓ |
| SACC-0015411 | 5.78 | 7.97 | 10.1 | 0.309 | ✓ |
| SACC-0011565 | 0.0417 | >25.0 | >25.0 | 1.22 | |
| SACC-0011206 | >25.0 | 6.54 | 17.5 | 0.985 | |
| SACC-0010978 | >25.0 | 9.82 | 0.0677 | 12.2 | |
| SACC-0005102 | 1.08 | 0.0832 | 0.485 | 0.177 | |

A ✓ indicates a validated antagonist.

TABLE 5
Validation of 7 analogs of the most potent antagonist identified (SACC-0064443) in the dose-dependent assay. Dose-
response curves (FIGS. 5A-5H) were generated from the fluorescence responses of the recombinant tick kinin receptor
(BMLK3) and vector-only cell (V/O) in the dual-addition assay (described in Example 1 in the Materials and Method
section). The Relative Fluorescence Units (RFU) in the Y-axis was normalized by subtracting the background signal.

| Molecule Name | BMLK3 EC50 (μM) | BMLK3 antagonist IC50 (μM) | Vector only EC50 (μM) | V/O antagonist IC50 (μM) | |
|---|---|---|---|---|---|
| SACC-0412066 | <0.0285 | 3.22 | >25.0 | 1.04 | * |
| SACC-0412065 | 2.67 | 0.0807 | 1.25 | 0.209 | |
| SACC-0412064 | 13 | 0.118 | 17.4 | 1.47 | |
| SACC-0412063 | 24.2 | 0.627 | 1.21 | >25.0 | |
| SACC-0412062 | 6.61 | 1.18 | 6.26 | 7.36 | * |
| SACC-0412061 | 0.978 | 0.509 | 5.5 | 2.71 | |
| SACC-0412060 | 9.57 | 1.89 | 9.27 | 0.538 | * |
| SACC-0064443 | 2.01 | 0.666 | 9.13 | >25.0 | * |

A * indicates the IC₅₀s of molecules validated as full antagonists

TABLE 6
Summary list of 36 validated antagonist hits. Dose-response curves (not shown) were generated from the fluorescence
responses of the recombinant tick kinin receptor (BMLK3) and vector-only cell (V/O) in the dual-addition assay
(described in the Materials and Method). The Relative Fluorescence Units (RFU) in the Y-axis was normalized
by subtracting the background signal. * Human dermal fibroblasts cells were used for cytotoxicity assay. A
** indicates molecules caused >10% cell death in the assay which was above the cutoff point.

| | BMLK3 | BMLK3 | Vector only | V/O Antagonist | | | Inhibitory | |
|---|---|---|---|---|---|---|---|---|
| | EC₅₀ | IC₅₀ | EC₅₀ | IC₅₀ | HDF average % | Partial/Full | Efficacy at | |
| Molecule Name | (μM) | (μM) | (μM) | (μM) | growth inhibition | antagonist | 12 μM | |
| SACC-0024648 | 0.346 | 2.11 | 0.665 | 6.82 | −8.08333 | F | 107.63% | |
| SACC-0048555 | 8.88 | 1.51 | 12.8 | 3.59 | −9.76667 | F | 107.39% | |
| SACC-0125715 | >25.0 | 1.55 | 14.3 | 0.711 | −1.92 | F | 104.16% | |
| SACC-0105544 | 7.26 | 3.33 | 11.4 | 0.816 | 6.956667 | F | 92.25% | |
| SACC-0412060 | 9.57 | 1.89 | 9.27 | 0.538 | 6.873333 | F | 91.75% | |
| SACC-0125713 | 12.1 | 4.95 | 0.0558 | 0.462 | 7.543333 | F | 91.08% | |
| SACC-0053274 | 0.808 | 1.28 | 0.849 | 4.28 | 11.04667 | F | 90.98% | ** |
| SACC-0064443 | 2.01 | 0.666 | 9.13 | >25.0 | 2.623333 | F | 89.44% | |
| SACC-0015411 | 5.78 | 7.97 | 10.1 | 0.309 | 10.57667 | F | 85.08% | ** |
| SACC-0113072 | 2.79 | 2.33 | 1.95 | 2.59 | −3.32333 | P | 82.45% | |
| SACC-0029037 | 4.1 | >25.0 | 12.4 | 7.67 | 10.19 | F | 79.39% | ** |
| SACC-0412066 | <0.0285 | 3.22 | >25.0 | 1.04 | 2.02 | F | 79.17% | |
| SACC-0089495 | 3.04 | 6.53 | 9.06 | 5.99 | −2.81667 | P | 71.34% | |
| SACC-0099442 | 5.4 | 7.17 | 10.8 | 1.12 | −1.16 | F | 71.31% | |
| SACC-0039590 | 2.27 | >25.0 | 1.63 | 5.58 | −13.23 | F | 69.87% | |
| SACC-0005314 | 0.956 | 3.09 | 0.474 | 0.434 | 1.676667 | P | 67.63% | |
| SACC-0126875 | 5.73 | 8.51 | 4.61 | >25.0 | 7.476667 | P | 59.47% | |
| SACC-0018618 | >25.0 | 4.29 | >25.0 | >25.0 | 7.326667 | F | 55.77% | |
| SACC-0033457 | 0.122 | | | | 8.023333 | F | 55.73% | |
| SACC-0027607 | <0.0285 | 0.483 | <0.0285 | >25.0 | 1.74 | P | 51.54% | |
| SACC-0412062 | 6.61 | 1.18 | 6.26 | 7.36 | 8.116667 | F | 50.31% | |
| SACC-0412061 | 0.978 | 0.509 | 5.5 | 2.71 | −1.01333 | P | 48.16% | |
| SACC-0121252 | 0.177 | >25.0 | >25.0 | 2.53 | 4.83 | F | 42.68% | |
| SACC-0054132 | <0.0285 | 3.69 | 2.56 | 0.207 | 9.036667 | P | 37.34% | |
| SACC-0050177 | 7.08 | >25.0 | >25.0 | 12.3 | 38.55333 | P | 35.48% | ** |
| SACC-0006795 | >25.0 | 17.3 | <0.0285 | >25.0 | −1.77333 | F | 33.05% | |
| SACC-0057260 | 0.54 | 6.63 | 1.05 | >25.0 | −5.02333 | P | 32.26% | |
| SACC-0034373 | 1.96 | 6.22 | 2.49 | >25.0 | −7 | P | 26.16% | |
| SACC-0123851 | 6.57 | 15.3 | 6.85 | 2.36 | 2.85 | P | 16.24% | |
| SACC-0412065 | 2.67 | 0.0807 | 1.25 | 0.209 | 6.54 | P | 14.72% | |
| SACC-0101074 | 11 | 10.9 | 2.58 | 2.95 | −4.72667 | P | 11.92% | |
| SACC-0021424 | >25.0 | 13.6 | <0.0285 | >25.0 | −1.69333 | P | 11.60% | |
| SACC-0010666 | 0.914 | 0.808 | 1.35 | 14.9 | −4.62 | P | 4.47% | |
| SACC-0412063 | 24.2 | 0.627 | 1.21 | >25.0 | 2.276667 | P | 0.61% | |
| SACC-0412064 | 13 | 0.118 | 17.4 | 1.47 | −7.06667 | P | 0.31% | |
| SACC-0058222 | >25.0 | 15.4 | >25.0 | 11.8 | −8.05333 | P | −0.42% | |

TABLE 7
Structure similarity analysis of 36 validated antagonists of the tick kinin receptor. Molecules in the same cluster have a common scaffold.

| | | Structure | Formula |
|---|---|---|---|
| | | Cluster | of |
| Molecule Name | SMILES (Simplified molecular-input line-entry system) | Group | Cluster |
| SACC-0064443 | C1C1═CC═C(\C═C2\SC(═O)N(NS(═O)(═O)C3═CC═CC═C3)C2═S)C═C1 | 1 | XCI |
| SACC-0412060 | O═C1N(NS(═O)(═O)c2ccccc2)C(═S)SC1═Cc1ccccc1 | 1 | XCI |
| SACC-0412061 | Cc1ccc(C═C2SC(═S)N(NS(═O)(═O)c3ccc(C)cc3)C2═O)cc1 | 1 | XCI |
| SACC-0412062 | Cc1ccc(cc1)S(═O)(═O)NN1C(═S)SC(═Cc2cccc3ccccc23)C1═O | 1 | XCI |
| SACC-0412063 | Cc1ccc(cc1)S(═O)(═O)NN1C(═S)SC(═Cc2ccc(F)cc2)C1═O | 1 | XCI |
| SACC-0412064 | Cc1ccc(cc1)S(═O)(═O)NN1C(═S)SC(═Cc2ccc(cc2)C(O)═O)C1═O | 1 | XCI |
| SACC-0412065 | Cc1ccc(cc1)S(═O)(═O)NN1C(═S)SC(═Cc2ccc(Cl)cc2Cl)C1═O | 1 | XCI |
| SACC-0412066 | CCOc1cc(C═C2SC(═S)N(NS(═O)(═O)c3ccc(C)cc3)C2═O)ccc1O | 1 | XCI |
| SACC-0126875 | OC(═O)COC1═C(\C═N\NC2═C3N═CC═CC3═CC═C2)C═C(Br)C═C1 | 2 | N/A |
| SACC-0125713 | OC(═O)C1═CC═CC(NC2═N[C@H]3C═CC(Br)═C[C@@H]3C(═N2)C2═CC═CC═C2)═C1 | 3 | XCII |
| SACC-0125715 | OC(═O)C1═CC═CC(NC2═NC3C═CC(Br)═CC3C(═N2)C2═CC═CC═C2)═C1 | 3 | XCII |
| SACC-0123851 | O═S(═O)(NC1═C2C═C(SC2═NC═N1)C1═CC═CC═C1)C1═CC2═C(OCCO2)C═C1 | 4 | N/A |
| SACC-0121252 | O═C1C═CC(NC2═CC═C(C═C2)N(═O)═O)C1NC1═CC═C(C═C1)N(═O)═O | 5 | N/A |
| SACC-0113072 | O\N═C(\CCCCCCC\C(═N/O)C1═CC═CC═C1)C1═CC═CC═C1 | 6 | N/A |
| SACC-0105544 | FC1═CC═CC(C1)═C1COC1═CC═C(\C═N\N2C(S)═NN═C2C(F)(F)F)C═C1 | 7 | N/A |
| SACC-0101074 | FC(F)(F)C1═CC═CC(═C1)C1═CC═C(O1)C(═O)[C@@H(C#N)C1═NC2═CC═CC═C2C(═O)N1 | 8 | N/A |
| SACC-0099442 | CSC1═NC2═CC═C(NS(═O)(═O)C3═CC═C(C1)S3)C═C2S1 | 9 | N/A |
| SACC-0057260 | CCOC(═O)C1═CC═C(C═C1)S(═O)(═O)NC1═CC═C2N═C(SC)SC2═C1 | 10 | XCIII |
| SACC-0089495 | COC1═CC═C(C═C1)S(═O)(═O)NC1═CC(SC2═NC3═CC═CC═C3S2)═C(O)C═C1 | 10 | XCIII |
| SACC-0058222 | CCOC(═O)N\N═C(\C1═C(O)N(N═C1C)C1═CC═CC═C1)C1═CC═C(C1)C═C1 | 11 | N/A |
| SACC-0054132 | CCN1C═C\C(═C/C═C/C2═CC═[N+](CC)C3═CC═CC═C23)C2═CC═CC═C12 | 12 | N/A |
| SACC-0053274 | CCN1\C(═C\C2═[N+](CC)C3═CC═C(C)C═C3C═C2)C═CC2═CC(C)═CC═C12 | 13 | N/A |
| SACC-0050177 | CCCCCCCCN1C2═C(CCC2)C(═N)C2═C1CCCC2 | 14 | N/A |
| SACC-0048555 | CCCC[N+]1═C(\C═C2\C═CN(CC)C3═C2C═C(C)C═C3)C═CC2═CC═CC═C12 | 15 | N/A |
| SACC-0039590 | CC1═CSC2═NC═NC(NCCC3═CC═C(C1)C═C3)═C12 | 16 | N/A |
| SACC-0034373 | CC1═CC═C(C═C1)S(═O)(═O)NC1═CC═C2N═C(N)SC2═C1 | 17 | N/A |
| SACC-0033457 | CC1═CC═C(C═C1)C1═CSC2═NC═NC(NCC3═CC═CO3)═C12 | 18 | N/A |
| SACC-0029037 | CC1═C(SC2═NC═NC(NC3CCN(CC3)C(═O)C3═CC═CO3)═C12)C1═CC═CC═C1 | 19 | N/A |
| SACC-0027607 | CC1═C(CON2N═NC3═CC═C(C═C23)C(F)(F)F)C(C)═NO1 | 20 | N/A |
| SACC-0024648 | CC1═C(\C(═N/NC2═NC3═CC═CC═C3S2)C2═CC═CC═C2)C(═O)N(N1)C1═CC═CC═C1 | 21 | N/A |
| SACC-0021424 | CC(NNC(═S)NCC1═CC═CC═C1)═C1CCCCC1 | 22 | N/A |
| SACC-0018618 | CC(C)CC\C(C)═N\N═C(/S)NCC1═CC═CC═C1 | 23 | N/A |
| SACC-0015411 | CC(C)(C)C1═CC═C(\C═N\N2C(S)═NN═C2C(F)(F)F)C═C1 | 24 | N/A |
| SACC-0010666 | C\N═C1\NC2═CC(═C(C═C2N\C\1═N/C)N(═O)═O)N(═O)═O | 25 | N/A |
| SACC-0006795 | C[C@@](O)(C#CC1═CC═CC═C1)C1═CC═C(N2CCCCC2)C(F)═C1 | 26 | N/A |
| SACC-0005314 | BrC1═CC═C(S1)S(═O)(═O)NC1═C2C═C(SC2═NC═N1)C1═CC═CC═C1 | 27 | N/A |

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An arthropod kinin receptor agonist or antagonist comprising a small molecule selected from:
a small molecule as disclosed in Tables 2-7 with one or more substitutions;
a small molecule having a structural similarity of about 50% or greater to the small molecules disclosed in Tables 2-7; and
a small molecule analog of an arthropod kinin having a normalized percent activation of greater than about 40% as defined by Equation (II) or inhibitory activity greater than about 40% as defined by Equation (III).

2. The arthropod kinin receptor agonist or antagonist of claim 1 having a structural similarity of about 60% or greater to the small molecules disclosed in Tables 2-7.

3. The arthropod kinin receptor agonist or antagonist of claim 1 having a structural similarity of about 75% or greater to the small molecules disclosed in Tables 2-7.

4. The arthropod kinin receptor agonist or antagonist of claim 1 having a structural similarity of about 90% or greater to the small molecules disclosed in Tables 2-7.

5. The arthropod kinin receptor agonist or antagonist of claim 1 having a structural similarity of about 95% or greater to the small molecules disclosed in Tables 2-7.

6. The arthropod kinin receptor antagonist of claim 1 having an inhibitory activity greater than about 50% as defined by Equation (III).

7. The arthropod kinin receptor antagonist of claim 1 having an inhibitory activity greater than about 60% as defined by Equation (III).

8. The arthropod kinin receptor agonist of claim 1 having a normalized percent activation of greater than about 50% as defined by Equation (II).

9. The arthropod kinin receptor agonist of claim 1 having a normalized percent activation of greater than about 60% as defined by Equation (11).

10. A method of identifying an arthropod kinin receptor agonist or antagonist, comprising:
culturing cells expressing an arthropod kinin receptor;
administering to the cells an assay buffer;
incubating the cells with control agonist and detecting the binding of the control agonist to the receptor;
incubating the cells with a small molecule for a sufficient time and detecting the binding of the small molecule to the receptor.

11. The method of claim 10, wherein the control agonist is a small molecule as disclosed in Table 2.

12. The method of claims 10 or 11, wherein the detection of binding is a calcium mobilization assay.

13. The method of any one of claims 10-12, wherein the agonist and/or antagonist is labeled.

14. The method of claim 13, wherein the label is a radioactive isotope, fluorescent dye, amine, or chemical tag.

15. A pharmaceutical composition, comprising:
a small molecule antagonist selected from:
a small molecule antagonist as disclosed in Tables 2-7;
a small molecule antagonist as disclosed in Tables 2-7 with one or more substitutions;
a small molecule antagonist having a structural similarity of about 50% or greater to the small molecules disclosed in Tables 2-7; and
a small molecule antagonist an arthropod kinin having a normalized percent activation of greater than about 40% as defined by Equation (II) or inhibitory activity greater than about 40% as defined by Equation (III); and
optionally a pharmaceutical accepted carrier, fungicides, bactericides, acaricides, molluscicides, nematicides, insecticides, microbiological agents, and/or beneficial organisms.

16. The pharmaceutical composition of claim 15, further comprising a safener.

17. The pharmaceutical compositions of claims 15 or 16, further comprising a plant extract.

18. The pharmaceutical composition of any one of claims 15-17, wherein the small molecule has one or more substitutions.

19. A method of treating or preventing arthropod-infection of a subject comprising: administrating an effective dose of the compound of an antagonist of any one of claims 1-7 or composition of any one of claims 15-18, optionally wherein the method further comprises concurrent or sequential administration with another fungicides, bactericides, acaricides, molluscicides, nematicides, insecticides, microbiological agents, and/or beneficial organisms.

20. The method of claim 19, wherein the subject is a mammal.

21. The method of claim 20, wherein the subject is a human.

22. The method of claim 20, wherein the subject is a breeding animal, zoo animal, laboratory animal, experimental animal, or domestic animal.

23. The method of claim 19, wherein the administration is enteral, parenteral, dermal, or nasal.

24. The method of claim 19, wherein the administration is dipping, spraying, or misting.

25. An agricultural composition, comprising:
a small molecule antagonist of any one of claims 1-7 or composition of any one of claims 15-18; and
optionally an agriculturally accepted carrier, fungicides, bactericides, acaricides, molluscicides, nematicides, insecticides, microbiological agents, beneficial organisms, herbicides, fertilizers, bird repellents, phytotonics, sterilants, safeners, semiochemicals and/or plant growth regulators.

26. The agricultural composition of claim 25, further comprising a safener.

27. The agricultural compositions of claims 25 or 26, further comprising a plant extract.

28. The agricultural compositions of any one of claims 25-27, wherein the small molecule has a substitution.

29. A method of treating or preventing the spreading of arthropods comprising:
administrating an effective dose of a composition of any one of claims 25-28, optionally wherein the method further comprises concurrent or sequential administration with another fungicides, bactericides, acaricides, molluscicides, nematicides, insecticides, microbiological agents, beneficial organisms, herbicides, fertilizers, bird repellents, phytotonics, sterilants, safeners, semiochemicals and/or plant growth regulators.

30. The method of claim 29, wherein the composition is administered to a plant or plant part.

31. The method of claim 30, wherein the plant part is a shoot, leaf, flower, seed, and/or root.

32. The method of any one of claims 30-31, wherein the administration is foliar.

33. The method of claim 29, wherein the composition is administered to a vacant pasture.

34. The method of claim 33, wherein administration is spraying, atomizing, irrigating, evaporating, dusting, fogging, broadcasting, foaming, painting, spreading-on, injecting, watering (drenching), or drip irrigating.

35. A method of detecting a change in a cell expressing an arthropod kinin receptor, comprising:
culturing cells expressing an arthropod kinin receptor;
administering to the cells an assay buffer;
incubating the cells with a small molecule as disclosed in Tables 2-7 for a sufficient time and detecting the binding of the small molecule to the receptor.

36. The method of claim 35, wherein the small molecule is labeled.

37. The method of claim 36, wherein the label is a radioactive isotope, fluorescent dye, amine, or chemical tag.

38. The method of any one of claims 35-37, wherein the small molecule has a substitution.

39. A method of controlling a parasite comprising administering an effective dose of a compound of any one of claims 1-8 or a composition of any one of claims 15-18 to the parasite for a sufficient time.

40. The method of 39, wherein the method further comprises concurrent or sequential administration with another fungicides, bactericides, acaricides, molluscicides, nematicides, insecticides, microbiological agents, beneficial organisms, or plant extract.

41. An animal bait, comprising:
a small molecule of any one of claims 1-7 or composition of any one of claims 15-18; and
optionally an anti-oxidizing agent, a preservative, a coloring agent, a flavonng agent, and/or a feed attractant.

42. The animal bait of claim 41, wherein the animal bait is liquid.

43. The animal bait of claims 41 or 42, further comprising an insecticide.

44. The animal bait of claims 42 or 43, further comprising a volatile attractant.

45. The animal bait of any one of claims 42-44, further comprising a sugar.

46. The animal bait of any one of claims 41-45, wherein the small molecule is an antifeedant or a feeding enhancer.

47. The animal bait of claim 41, wherein the animal bait is solid.
